(19) United States
(12) Patent Application Publication
(10) Pub. No.: US 2024/0116988 A1
(43) Pub. Date: Apr. 11, 2024

(54) ANTI-ROR1 MACROCYCLIC PEPTIDES AND COMPOSITIONS

(71) Applicant: BRISTOL-MYERS SQUIBB COMPANY, PRINCETON, NJ (US)

(72) Inventors: JENNIFER X. QIAO, ARLINGTON, MA (US); VING G. LEE, HAMILTON, NJ (US); TAMMY C. WANG, LAWRENCEVILLE, NJ (US); MICHAEL A. POSS, LAWRENCEVILLE, NJ (US)

(73) Assignee: BRISTOL-MYERS SQUIBB COMPANY, PRINCETON, NJ (US)

(21) Appl. No.: 18/454,489

(22) Filed: Aug. 23, 2023

Related U.S. Application Data
(60) Provisional application No. 63/373,225, filed on Aug. 23, 2022.

(51) Int. Cl. C07K 7/56; A61K 47/64

(52) U.S. Cl. CPC C07K 7/56; A61K 47/64

(57) ABSTRACT

There are disclosed novel anti-ROR1 macrocyclic peptides and their conjugates with general structure of formula (I), which can be used as ROR1 inhibitors.

### CROSS REFERENCE TO RELATED APPLICATION

This application claims the benefit of U.S. Provisional Application Serial No. 63/373225 filed Aug. 23, 2022 which is incorporated herein in its entirety.

This invention relates to novel anti-ROR1 macrocyclic peptides and their conjugates with general structure of formula (I), which can be used as ROR1 inhibitors. The macrocyclic peptides described in this invention bind to extracellular domain of human ROR1, and are capable of inhibiting human ROR1, thus are useful for the amelioration of various diseases related to ROR1 activity, including cancer and infectious diseases.

### BACKGROUND OF THE INVENTION

Receptor tyrosine kinases (RTK) are key regulators of cellular processes such as differentiation, proliferation, survival and migration and have a role in the development and progression of cancer. Deregulation of RTKs has been linked to the initiation and progression of cancers (Du and Lovely, Molecular Cancer (2018) 17:58).

Receptor tyrosine kinase-like orphan receptor 1 (ROR1) is a cell surface protein that mediates signals from its ligand, the secreted glycoprotein Wnt5a. Consistent with its role in influencing the fate of stem cells during embryogenesis, ROR1 expression is observed on invasive malignancies that revert to an embryonic transcriptional program, but is not observed on normal adult tissues, offering a favorable selectivity profile as a therapeutic target. ROR1 is commonly expressed on the malignant cells of patients with acute lymphoblastic leukemia (ALL), acute myeloid leukemia (AML), chronic lymphoblastic leukemia (CLL), mantle cell lymphoma (MCL), follicular lymphoma (FL), marginal zone lymphoma (MZL), diffuse large B-cell lymphoma (DLBCL), and Richter's transformation or Richter's syndrome (RS). ROR1 is also present on the cell surfaces of multiple solid tumors, where it appears to be a marker of cancer stem cells. Because it is not expressed to appreciable levels in healthy adult tissues, but displays high levels of expression in multiple hematological and solid tumors, ROR1 is an attractive target for tumor-specific therapy.

ROR1 presents multiple therapeutic modality possibilities, including antibody monotherapy, antibody-drug conjugates, radio conjugates, bispecific antibodies, and other multivalent biologics, which are targeting the extracellular domains of ROR1. Cirmtuzumab, a humanized antibody directed against ROR1, is undergoing clinical trials. A cirmtuzumab-MMAE antibody-drug conjugate is in clinical trials. On the other hand, small molecule kinase inhibitors of ROR1 have been recently described in the literature, e.g., US20210267994 A1.

In view of the critical role of ROR1 in cancer progression, there is a need for new and improved immune therapies that target ROR1 for treating cancer.

### SUMMARY OF THE INVENTION

This invention relates to novel anti-ROR1 macrocyclic peptides with general structure of formula (I) and their conjugates, which can be used as ROR1 inhibitors. The macrocyclic peptides described in this invention bind to human extracellular domain of human ROR1, and are capable of inhibiting human ROR1, thus are useful for the amelioration of various diseases related to ROR1 activity, including cancer and infectious diseases.

The first aspect of the present invention provides at least one compound of Formula (I)

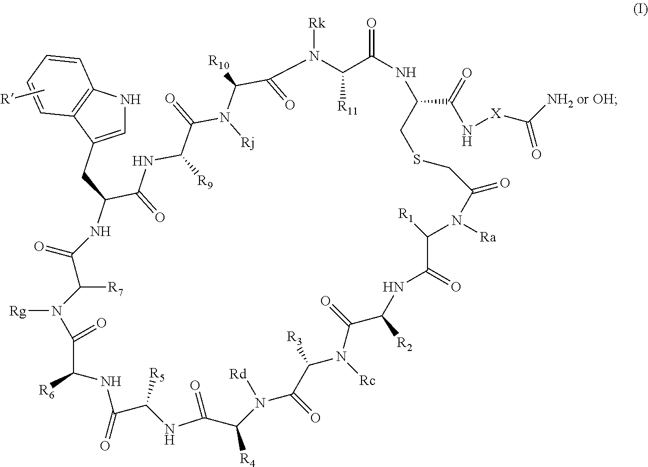

or a pharmaceutically acceptable salt thereof, wherein

R₁ is selected from the group consisting of C₁-C₆alkyl, hydroxyC₁-C₆alkyl, methoxyC₁-C₆alkyl, aminocarbonylC₁-C₆alkyl, aryl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆ alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;

R₂ is selected from the group consisting of hydrogen, C₁-C₆alkyl, arylC₁-C₆alkyl, wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; guanidinylC₁-C₆alkyl;

R₃ is selected from the group consisting of C₁-C₆alkyl, aminoC₁-C₆alkyl, arylC₁-C₆alkyl, and heteroarylC₁₋C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo; carboxyC1-C6alkyl, guanidinylC₁-C₆alkyl;

R₄ is selected from the group consisting of C₁-C₆alkyl, hydroxyC₁-C₆alkyl, arylC₁-C₆alkyl, and heteroarylC₁₋C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo; carboxyC1-C6alkyl, guanidinylC₁-C₆ alkyl;

R₅ is selected from the group consisting of hydrogen, C₁-C₆alkyl, hydroxyC₁-C₆alkyl, aminoC₁-C₆alkyl, guanidinylC₁-C₆alkyl, aminocarbonylC₁-C₆alkyl, arylC 1-C₆alkyl, and heteroarylC₁₋C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;

R₆ is selected from the group consisting of hydrogen, C₁-C₆alkyl, hydroxyC₁-C₆alkyl, aminoC₁-C₆alkyl, guanidinylC₁-C₆alkyl, aminocarbonylC₁-C₆alkyl, carboxyC₁-C₆alkyl, arylC₁-C₆alkyl, and heteroarylC₁₋C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl;

R₇ is selected from the group consisting of hydrogen, C₁-C₆alkyl, carboxyC₁-C₆alkyl, aminoC₁-C₆alkyl, guanidinylC₁-C₆alkyl, and heteroarylC₁₋C₆alkyl or halo;

R′ is selected from the group consisting of hydrogen, halo, C₁-C₃alkyl and cyano;

R₉ is selected from the group consisting of C₁-C₆alkyl, arylC₁-C₆alkyl, and heteroarylC₁₋C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;

R₁₀ is selected from C₃-C₆alkyl, arylC₁-C₆alkyl, and heteroarylC₁₋C₆alkyl; wherein the aryl part of the arylC₁-C₆alkyl is optionally substituted with one, two, three, four, or five groups independently selected from halo, nitro, amino, C₁₋C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;

R₁₁ is C₁-C₆alkyl;

Ra is hydrogen or methyl;

Rc is hydrogen or methyl; or Rc and R₃, together with the carbon atom to which they are attached, form a 5-6 ring heterocycle ring, wherein the heterocycle is optionally substituted with one or two groups independently selected from amino, aminocarbonyl, carboxy, carboxyC₁-C₆alkyl, carboxymethoxy, cyano, fluoro, hydroxy, methoxy, methyl, methylcarbonylamino, and trifluoromethyl;

Rd is hydrogen or methyl;

Rg is hydrogen or methyl;

Rj is hydrogen or methyl;

X is selected from the group consisting of-

CR₁₃R₁₃′CONHCR₁₄R₁₄′CONHCR₁₅R₁₅′, wherein R₁₃, R₁₄, R₁₅ is independently selected from hydrogen, or any natural or unnatural amino acid side chains, and R₁₃′, R₁₄′, and R₁₅′ is interdependently selected from hydrogen or C₁-C₆alkyl; alternatively, X is —(CH₂CH₂O)ₙ—, wherein n=1-13; Alternatively X is —(CH₂CH₂O)ₙ—CONH—CR₁₆R₁₆′; wherein n=1-13 and R₁₆, R₁₆′ is independently selected from hydrogen, aminoC₁-C₄alkyl, carboxyC₁-C₂ alkyl, HSC₁-C₂alkyl;

In one embodiment of the invention, there is disclosed a compound of formula (I), or the pharmaceutically acceptable salt thereof, wherein Ru is selected from C₁-C₄alkyl, hydroxyC₁-C₃ alkyl, aminocarbonylC₁-C₂alkyl, aryl, arylC₁-C₂alkyl, and heteroarylC₁₋C₂alkyl; wherein the aryl part of the arylC₁-C₂alkyl is phenyl, and is optionally substituted with one, or two groups independently selected from halo, amino, C₁-C₂alkyl, aminocarbonyl, hydroxy, aminoC₁-C₂alkyl, trifluoromethyl, oxotrifluoromethyl, and cyano; and wherein the heteroaryl part of the heteroarylC₁-C₂alkyl is indolyl or imidazole, and is optionally substituted with one or two groups independently selected from methyl, fluoro or chloro;

In another embodiment of the invention, there is disclosed a compound of formula (I), or the pharmaceutically acceptable salt thereof, wherein R₂ is selected from hydrogen, C1-C4alkyl, guanidinylC₃-C₄ alkyl, arylC₁-C₂alkyl, wherein the aryl part of the arylC₁-C₆alkyl is phenyl and is optionally substituted with one or two groups independently selected from fluoro, amino, C₁-C₃ alkyl, hydroxy, aminoC₃-C₄ alkyl, and aminocarbonylC1-C2alkyl;

In another embodiment of the invention, there is disclosed a compound of formula (I), or the pharmaceutically acceptable salt thereof, wherein R3 is selected from C₁-C₄alkyl, aminoC₃-C₄alkyl, arylC₁-C₂alkyl, and heteroarylC₁₋C₂alkyl; wherein the aryl part of the arylC₁-C₆alkyl is phenyl and is optionally substituted with one or two groups independently selected from fluoro, amino, C₁-C₃ alkyl, aminocarbonyl, hydroxy, aminoC₁-C₃alkyl, trifluoromethyl, oxotrifluoromethyl, carboxy, cyano, carboxyC₁-C₂alkyl, and carboxymethoxy; carboxyC1-C2alkyl, guanidinylC₃-C₄ alkyl; alternatively R₃ and Rc, together with the carbon atom to which they are attached, form a pyrrolidine ring, which is optionally substituted with one or two groups independently selected from amino, aminocarbonyl, carboxy, carboxyC₁-C₆alkyl, carboxymethoxy, cyano, fluoro, hydroxy, methoxy, methyl, methylcarbonylamino, and trifluoromethyl;

In another embodiment of the invention, there is disclosed a compound of formula (I), or the pharmaceutically acceptable salt thereof, wherein R₄ is selected from C₁-C₄alkyl, hydroxyC₁-C₂alkyl, arylC₁-C₂alkyl, and heteroarylC₁₋C₂alkyl; wherein the aryl part of the arylC₁-C₆alkyl is optionally substituted with one or two groups independently selected from hydrogen, fluoro, amino, C₁-C₃alkyl, aminocarbonyl, hydroxy, aminoC₁-C₃alkyl, trifluoromethyl, oxotrifluoromethyl, carboxy, cyano, carboxyC₁-C₂alkyl, and carboxymethoxy;

In another embodiment of the invention, there is disclosed a compound of formula (I), or the pharmaceutically acceptable salt thereof, wherein R5 is hydrogen, C₁-C₄alkyl, hydroxyC₁-C₂alkyl, aminoC₁-C₄alkyl, guanidinylC₃-C₄ alkyl, aminocarbonylC₁-C₂alkyl, arylC₁-C₂alkyl, and heteroarylC₁₋C₂alkyl; wherein the aryl part of the arylC₁-C₆alkyl is optionally substituted with one or two groups independently selected from hydrogen, fluoro, amino, C₁-C₃alkyl, aminocarbonyl, hydroxy, aminoC₁-C₃alkyl, trifluoromethyl, oxotrifluoromethyl, carboxy, cyano, carboxyC₁-C₂alkyl, and carboxymethoxy;

In another embodiment of the invention, there is disclosed a compound of formula (I), or the pharmaceutically acceptable salt thereof, wherein R₆ is hydrogen, C₁-C₄alkyl, hydroxyC₁-C₂ alkyl, aminoC₁-C₄alkyl, guanidinylC₃-C₄alkyl, aminocarbonylC₁-C₂alkyl, phenylC₁-C₂alkyl, and indolylC₁₋C₂alkyl; wherein benzyl or indolyl is optionally substituted with one or more groups independently selected from hydroxy, amino, aminocarbonyl, carboxy.

In another embodiment of the invention, there is disclosed a compound of formula (I), or the pharmaceutically acceptable salt thereof, wherein R₇ is selected from hydrogen, C₁-C₄alkyl, aminoC₁-C₄alkyl, guanidinylC₃-C₄alkyl, carboxyC₁-C₂alkyl, indolylC₁₋C₆alkyl;

In another embodiment of the invention, there is disclosed a compound of formula (I), or the pharmaceutically acceptable salt thereof, wherein R₉ is isopropylmethyl or arylmethyl; wherein the aryl part of the arylmethyl is optionally substituted with one or two groups independently selected from hydroxy, aminocarbonyl, halo, and C₁-C₃alkyl and trifluoromethyl.

In another embodiment of the invention, there is disclosed a compound of formula (I), wherein R₁₀ is C₃-C₆alkyl, phenylmethyl or indolylmethyl, wherein the phenyl or indolyl part is optionally substituted with one, two, or three fluoro, methyl, or trifluoromethyl.

In another embodiment of the invention, there is disclosed a compound of formula (I), or the pharmaceutically acceptable salt thereof, wherein R₁ is benzyl, indolylmethyl, —CH₂CH₂CONH₂, imidazolylmethyl, isopropylmethyl, and 2-naphthylmethyl.

In another embodiment of the invention, there is disclosed a compound of formula (I), or the pharmaceutically acceptable salt thereof, wherein R₂ is selected from C1-C4alkyl, guanidinylpropyl, 4-hydroxyphenylmethyl.

In another embodiment of the invention, there is disclosed a compound of formula (I), or the pharmaceutically acceptable salt thereof, wherein R₃ is selected from C₁-C₄alkyl, benzyl, 4-hydroxyphenyl, aminobutyl, guanidinylpropyl; alternatively, R₃ and Rc, together with the carbon atom to which they are attached, form a pyrrolidine ring;

In another embodiment of the invention, there is disclosed a compound of formula (I), or the pharmaceutically acceptable salt thereof, wherein R₄ is methyl, isopropyl, benzyl, or indolylmethyl.

In another embodiment of the invention, there is disclosed a compound of formula (I), or the pharmaceutically acceptable salt thereof, wherein R₅ is C₁-C₄alkyl, benzyl, 4-hydroxyphenylmethyl, or hydroxymethyl.

In another embodiment of the invention, there is disclosed a compound of formula (I), or the pharmaceutically acceptable salt thereof, wherein R₆ is hydrogen, C₁-C₄alkyl, hydroxymethyl, carboxymethyl, carboxyethyl, guanidinylpropyl, aminocarbonylmethyl, aminocarbonylethyl, aminoC₁-C₄alkyl, benzyl, imidazolyl, indolyl, or 4-hydroxyphenylmethyl.

In another embodiment of the invention, there is disclosed a compound of formula (I), or the pharmaceutically acceptable salt thereof, wherein R₇ is hydrogen, methyl, isopropyl, or guanidinylpropyl.

In another embodiment of the invention, there is disclosed a compound of formula (I), or the pharmaceutically acceptable salt thereof, wherein, R₉ is isopropyl, isopropylmethyl, benzyl, or 4-hydroxyphenylmethyl.

In another embodiment of the invention, there is disclosed a compound of formula (I), or the pharmaceutically acceptable salt thereof, wherein R₁₀ is isopropylmethyl, n-butyl, —CH(CH₃)CH₂CH₃, benzyl, 4-hydoxyphenylmethyl, or indolylmethyl.

In another embodiment of the invention, there is disclosed a compound of formula (I), or the pharmaceutically acceptable salt thereof, wherein, R₁₁ is hydrogen, methyl, isopropyl, isopropylmethyl, or n-butyl.

In another embodiment of the invention, there is disclosed a compound of formula (I), or the pharmaceutically acceptable salt thereof, wherein

R₁ is benzyl or indolylmethyl,

R₂ is 4-hydorxyphenylmethyl.

R₃ is selected from isopropylmethyl, indolylmethyl, guanidinylpropyl, or aminobutyl;

alternatively, R₃ and Rc, together with the carbon atom to which they are attached,

form the pyrrolidine ring,

R₄ is isopropyl or hydroxymethyl,

R₅ is hydroxymethyl or 4-hydroxyphenylmethyl,

R₆ is selected from hydroxymethyl, guanidinylpropyl, aminocarbonylmethyl, aminocarbonylethyl, carboxyethyl, or 4-hydroxyphenylmethyl,

R₇ is hydrogen, or methyl,

R₉ is 4-hydroxyphenylmethyl,

R₁₀ is selected from n-butyl, isopropylmethyl, or benzyl,

R₁₁ is n-butyl or isopropylmethyl.

In another embodiment of the invention, there is disclosed a compound of formula (I), or the pharmaceutically acceptable salt thereof, wherein R₁₆ is HS-methyl-, aminoC₁-C₄ alkyl, or carboxyC₁-C₂, which can be conjugated to a cytotoxic payload (D) through a linker (L′) to form a general structure (II), one of such examples is illustrated as follows:

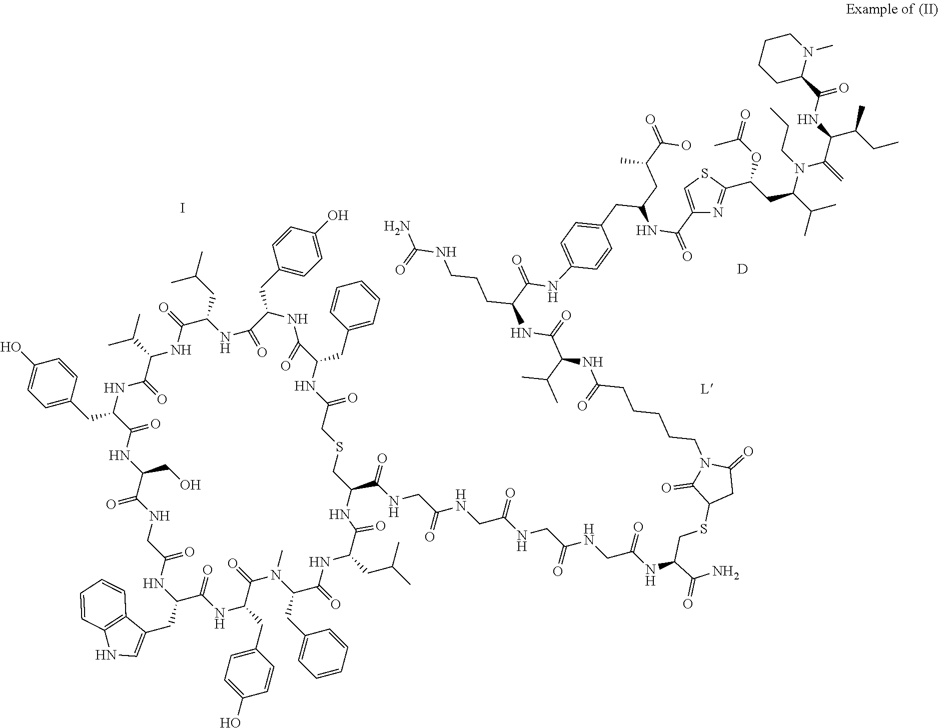

In another embodiment of the invention, there is disclosed a compound of formula (I), or the pharmaceutically acceptable salt thereof, wherein the compound is selected from the compounds listed in Table 1.

In another embodiment of the invention, there is disclosed a pharmaceutical composition comprising any one of the compounds of the invention, or a pharmaceutically acceptable. salt thereof, for use as an inhibitor of tyrosine kinase ROR1 activity in a mammal; preferably a human

In another embodiment of the invention, there is disclosed a method of enhancing, stimulating, and/or increasing an immune response in a subject in need thereof, wherein the method comprises administering to the subject a therapeutically effective amount of any one of the compounds of the invention or a pharmaceutically acceptable salt thereof. and optionally a pharmaceutically acceptable excipient.

In another embodiment of the invention, there is disclosed a method of binding the extracellular domain(s) of human ROR1 in a subject, wherein the method comprises administering to the subject a therapeutically effective amount of any one of the compounds or a pharmaceutically acceptable salt thereof.

In another embodiment of the invention, there is disclosed a compound of formula (Γ) or (I″), or a pharmaceutically acceptable salt thereof, for use in the treatment of a malignant hyperproliferative disorder. Examples of malignant hyperproliferative disorders include, but are not limited to, hematological tumors such as chronic lymphocytic leukemia (CLL), acute myeloid leukemia (AML), acute lymphoblastic leukemia (ALL) or mantle cell lymphoma, and also solid tumors such as lung, ovarian, breast or pancreatic tumors.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of formula (I) or a pharmaceutically acceptable salt thereof” includes, but is not limited to, a compound of formula (I), two compounds of formula (I), a pharmaceutically acceptable salt of a compound of formula (I), a compound of formula (I) and one or more pharmaceutically acceptable salts of the compound of formula (I), and two or more pharmaceutically acceptable salts of a compound of formula (I).

The term “C₂-C₆alkenyl,” as used herein, refers to a group derived from a straight or branched chain hydrocarbon containing one or more carbon-carbon double bonds containing two to six carbon atoms.

The term “C₁-C₆alkoxy”, as used herein, refers to a C₁-C₆alkyl group attached to the parent molecular moiety through an oxygen atom.

The term “alkyl,” as used herein, refers to a group derived from a straight or branched chain saturated hydrocarbon containing carbon atoms. The term “alkyl” may be proceeded by “C #-C #” wherein the # is an integer and refers to the number of carbon atoms. For example, C₁-C₂alkyl contains one to two carbon atoms and C₁-C₃alkyl contains one to three carbon atoms.

The term “C₁-C₂alkylamino,” as used herein, refers to a group having the formula —NHR, wherein R is a C₁-C₂alkyl group.

The term “C₁-C₂alkylaminoC₁-C₆alkyl,” as used herein, refers to a C₁-C₂alkylamino group attached to the parent molecular moiety through a C₁-C₆alkyl group.

The term “C₁-C₆alkylcarbonyl,” as used herein, refers to a C₁-C₆alkyl group attached to the parent molecular moiety through a carbonyl group.

The term “C₁-C₂alkylcarbonylamino,” as used herein, refers to —NHC(O)Ra, wherein Ra is a C₁-C₆alkyl group.

The term “C₁-C₆alkylcarbonylamino,” as used herein, refers to —NHC(O)Ra, wherein Ra is a C₁-C₂alkyl group.

The term “C₁-C₂alkylcarbonylaminoC₁-C₆alkyl,” as used herein, refers to a C₁-C₂alkylcarbonylamino group attached to the parent molecular moiety through a C₁-C₆alkyl group.

The term “C₁-C₆alkylcarbonylaminoC₁-C₆alkyl,” as used herein, refers to a C₁-C₆alkylcarbonylamino group attached to the parent molecular moiety through a C₁-C₆alkyl group.

The term “C₁-C₆alkylheteroaryl,” as used herein, refers to a heteroaryl group

The term “C₁-C₆alkylheteroarylC₁-C₆alkyl,” as used herein, refers to a C₁-C₆alkylheteroaryl group attached to the parent molecular moiety through a C₁-C₆alkyl

The term “C₁-C₆alkylimidazolyl,” as used herein, refers to an imidazolyl ring substituted with one, two, or three C₁-C₆alkyl groups.

The term “C₁-C₆alkylimidazolylC₁-C2alkyl,” as used herein, refers to a C₁-C₆alkylimidazolyl group attached to the parent molecular moiety through a C₁-C₂ alkyl group.

The term “C₂-C₆alkynyl,” as used herein, refers to a group derived from a straight or branched chain hydrocarbon containing one or more carbon-carbon triple bonds containing two to six carbon atoms.

The term “C₂-C₆alkynylmethoxy,” as used herein, refers to a C₂-C₆alkynylmethyl group attached to the parent molecular moiety through an oxygen atom.

The term “C₂-C₆alkynylmethyl,” as used herein, refers to a C₂-C₆alkynyl group attached to the parent molecular moiety through a CH₂ group.

The term “amino,” as used herein, refers to —NH2.

The term “aminoC₁-C₃alkyl,” as used herein, refers to an amino group attached to the parent molecular moiety through a C₁-C₃ alkyl group.

The term “aminoC₁-C₆alkyl,” as used herein, refers to an amino group attached to the parent molecular moiety through a C₁-C₆alkyl group.

The term “aminobutyl,” as used herein, refers to —CH₂CH₂CH₂CH₂NH₂.

The term “aminocarbonyl,” as used herein, refers to an amino group attached to the parent molecular moiety through a carbonyl group.

The term “aminocarbonylC₁-C₂alkyl,” as used herein, refers to an aminocarbonyl group attached to the parent molecular moiety through a C₁-C₂alkyl group.

The term “aminocarbonylC₁-C₃alkyl,” as used herein, refers to an aminocarbonyl group attached to the parent molecular moiety through a C₁-C₃alkyl group.

The term “aminocarbonylC₁-C₆alkyl,” as used herein, refers to an aminocarbonyl group attached to the parent molecular moiety through a C₁-C₆alkyl group.

The term “aminocarbonylamino,” as used herein, refers to an aminocarbonyl group attached to the parent molecular moiety through an amino group.

The term “aminocarbonylaminoC₁-C₆alkyl,” as used herein, refers to an aminocarbonylamino group attached to the parent molecular moiety through a C₁-C₆alkyl group.

The term “aminocarbonylaminoC₂-C₆alkyl,” as used herein, refers to an aminocarbonylamino group attached to the parent molecular moiety through a C₂-C₆alkyl group.

The term “aminocarbonylaminomethyl,” as used herein, refers to an aminocarbonylamino group attached to the parent molecular moiety through a CH₂ group.

The term “aminocarbonylaminopropyl,” as used herein, refers to an aminocarbonylamino group attached to the parent molecular moiety through a CH₂CH₂CH₂ group.

The term “aminocarbonylmethyl,” as used herein, refers to an aminocarbonyl group attached to the parent molecular moiety through a CH₂ group.

The term “aminoethyl,” as used herein, refers to —CH₂CH₂NH₂.

The term “aminomethyl,” as used herein, refers to —CH₂NH₂.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₂alkyl,” as used herein, refers to an aryl group attached to the parent molecular moiety through a C₁-C₂alkyl group.

The term “arylmethyl,” as used herein, refers to an aryl group attached to the parent molecular moiety through a CH₂ group.

The term “carbonyl,” as used herein, refers to —C(O)—.

The term “carboxy”, as used herein, refers to —CO₂H.

The term “carboxyC₁-C₆alkoxy,” as used herein, refers to a carboxyC₁-C₆alkyl group attached to the parent molecular moiety through an oxygen atom.

The term “carboxyC₁-C₆alkyl”, as used herein, refers to a carboxy group attached to the parent molecular moiety through a C₁-C₆alkyl group.

The term “carboxymethoxy,” as used herein, refers to —OCH₂CO₂H.

The term “carboxymethyl,” as used herein, refers to —CH₂CO₂H.

The term “cyano,” as used herein, refers to —CN.

The term “cyanoC₁-C₆alkyl,” as used herein, refers to a cyano group attached to the parent molecular moiety though a C₁-C₆alkyl.

The term “C3-C₆cycloalkyl”, as used herein, refers to a saturated monocyclic or bicyclic hydrocarbon ring system having three to six carbon atoms and zero heteroatoms. The bicyclic rings can be fused, spirocyclic, or bridged. Representative examples of cycloalkyl groups include, but are not limited to, cyclopropyl, cyclopentyl, and cyclohexyl.

The term “C₃-C₈cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, cyclohexyl, cycloheptyl, and cyclooctyl.

The term “(C₃-C₆cycloalkyl)C₁-C₂alkyl”, as used herein, refers to a C₃-C₆cycloalkyl group attached to the parent molecular moiety through a C₁-C₂alkyl group.

The term “(C₃-C₆cycloalkyl)C₁-C₆alkyl”, as used herein, refers to a C₃-C₆cycloalkyl group attached to the parent molecular moiety through a C₁-C₆alkyl group.

The term “C₃-C₆cycloalkylcarbonyl,” as used herein, refers to a C₃-C₆cycloalkyl group attached to the parent molecular moiety through a carbonyl group.

The term “C₃-C₆cycloalkylcarbonylamino,” as used herein, refers to a C₃-C₆cycloalkylcarbonyl group attached to the parent molecular moiety through an amino group.

The term “C₃-C₆cycloalkylcarbonylaminoC₁-C₆alkyl,” as used herein, refers to a C₃-C₆cycloalkylcarbonylamino group attached to the parent molecular moiety through a C₁-C₆alkyl group.

The term “(C₃-C₆cycloalkyl)methyl”, as used herein, refers to a C₃-C₆cycloalkyl group attached to the parent molecular moiety through a CH₂ group.

The term “cyclopropylcarbonylaminoethyl,” as used herein, refers to —CH₂CH₂NHC(O)R, wherein R is a cyclopropyl group.

The term “difluorocyclohexylmethyl,” as used herein refers to a cyclohexyl group substituted with two fluoro groups that is attached to the parent molecular moiety through a CH₂ group.

The term “ethynylmethoxy,” as used herein, refers to —OCH₂C═CH.

The term “fluoroC₁-C₆alkyl,” as used herein, refers to a C₁-C₆alkyl group substituted by one, two, three, or four fluoro groups.

The term “fluoroC₁-C₆alkylcarbonyl,” as used herein, refers to a fluoroC₁-C₆alkyl group attached to the parent molecular moiety through a carbonyl group.

The term “fluoroC₁-C₆alkylcarbonylamino,” as used herein, refers to a fluoroC₁-C₆alkylcarbonyl group attached to the parent molecular moiety through an NH group.

The term “fluoroC₁-C₆alkylcarbonylaminoC₁-C₆alkyl,” as used herein, refers to a fluoroC₁-C₆alkylcarbonylamino group attached to the parent molecular moiety through a C₁-C₆alkyl group.

The term “fluoroC₄-C₆alkyl,” as used herein, refers to a C₄-C₆alkyl group substituted by one, two, three, or four fluoro groups.

The term “fluoroheterocyclyl,” as used herein, refers to a heterocycle group substituted with one, two, or three fluoro groups.

The term “fluoroheterocyclylC₁-C₆alkyl,” as used herein, refers to a fluoroheterocyclyl group attached to the parent molecular moiety through a C₁-C₆alkyl group.

The term “guanidinylC₁-C₆alkyl,” as used herein, refers to a NH₂C(NH)NH— group attached to the parent molecular moiety through a C₁-C₆alkyl group.

The term “guanidinylC₂-C₄alkyl,” as used herein, refers to a NH₂C(NH)NH— group attached to the parent molecular moiety through a C₂-C₄alkyl group.

The term “guanidinylC₂-C₆alkyl,” as used herein, refers to a NH₂C(NH)NH— group attached to the parent molecular moiety through a C₂-C₆alkyl group.

The terms “halo” and “halogen”, as used herein, refer to F, Cl, Br, or I.

The term “heteroaryl,” as used herein, refers to an aromatic five- or six-membered ring where at least one atom is selected from N, O, and S, and the remaining atoms are carbon.

The term “heteroaryl” also includes bicyclic systems where a heteroaryl ring is fused to a four- to six -membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl,” as used herein, refers to a heteroaryl group attached to the parent molecular moiety through a C₁-C₆alkyl group. The term “heteroarylmethyl,” as used herein, refers to a heteroaryl group attached to the parent molecular moiety through a CH 2 group.

The term “heterocyclyl,” as used herein, refers to a five-, six-, or seven-membered non-aromatic ring containing one, two, or three heteroatoms independently selected from nitrogen, oxygen, and sulfur. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure can be attached to the parent molecular moiety through any substitutable atom in the group. Examples of heterocyclyl groups include, but are not limited to, morpholinyl, piperazinyl, pyrrolidinyl, and thiomorpholinyl.

The term “heterocyclylC₁-C₆alkyl,” as used herein, refers to a heterocyclyl attached to the parent molecular moiety through a C₁-C₆alkyl group.

The term “hydroxy,” as used herein, refers to —OH.

The term “hydroxyC₁-C3alkyl,” as used herein, refers to a hydroxy group attached to the parent molecular moiety through a C₁-C₃ alkyl group.

The term “hydroxyC₁-C₆alkyl,” as used herein, refers to a hydroxy group attached to the parent molecular moiety through a C₁-C₆alkyl group.

The term “hydroxyaryl,” as used herein, refers to an aryl group substituted with one, two, or three hydroxy groups.

The term “hydroxyarylC₁-C2alkyl,” as used herein, refers to a hydroxyaryl group attached to the parent molecular moiety through a C₁-C₂alkyl group.

The term “indolylC₁-C₆alkyl,” as used herein, refers to an indolyl group attached to the parent molecular moiety through a C₁-C₆alkyl group.

The term “methoxy,” as used herein, refers to —OCH₃.

The term “methoxyC₁-C₂alkyl,” as used herein, refers to a methoxy group attached to the parent molecular moiety though a C₁-C₂alkyl group.

The term “methylcarbonylamino,” as used herein, refers to —NHC(O)CH₃.

The term “methylcarbonylaminobutyl,” as used herein, refers to —(CH₂)₄NHC(O)CH₃.

The term “methylcarbonylaminobutyl,” as used herein, refers to —(CH₂)₃NHC(O)CH₃.

The term “methylsulfanyl,” as used herein, refers to a —S—CH₃.

The term “methylsulfanylC₁-C₆alkyl,” as used herein, refers to a methylsulfanyl group attached to the parent molecular moiety through a C₁-C₆alkyl group.

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Receptor Tyrosine kinase-like orphan receptor 1”, “Protein ROR1”, “ROR1”, “hROR1”, are used interchangeably, and include variants, isoforms, species homologs of human ROR1, and analogs having at least one common epitope with ROR1. The complete ROR1 sequence can be found under GM:1mA Accession No NM_05012.

The term “treating” refers to i) inhibiting the disease, disorder, or condition, i.e., arresting its development; and/or ii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

### Peptide Synthesis

The macrocyclic peptides of the present disclosure can be produced by methods known in the art, such as they can be synthesized chemically, recombinantly in a cell free system, recombinantly within a cell or can be isolated from a biological source. Chemical synthesis of a macrocyclic peptide of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic peptides and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the a-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-Cl-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the peptide analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown below.

### Examples of Orthogonally Protected Amino Acids used in Solid Phase Synthesis

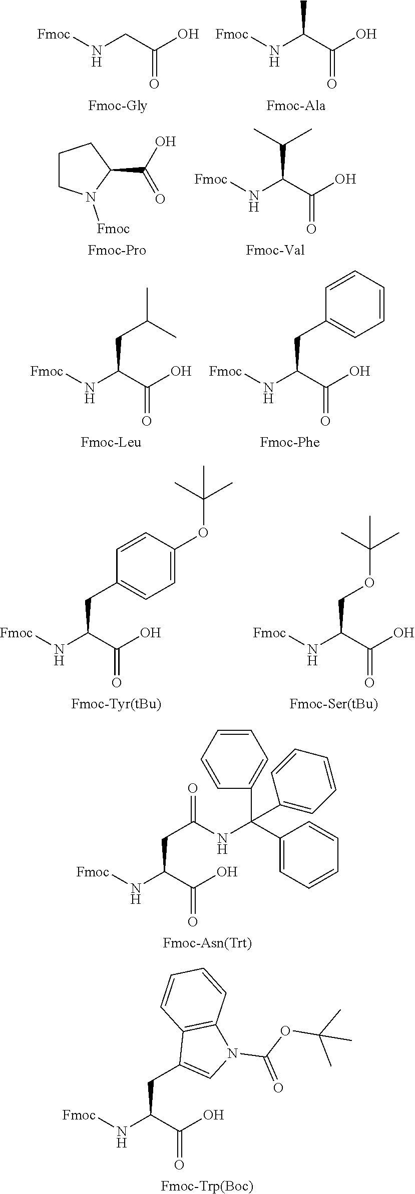

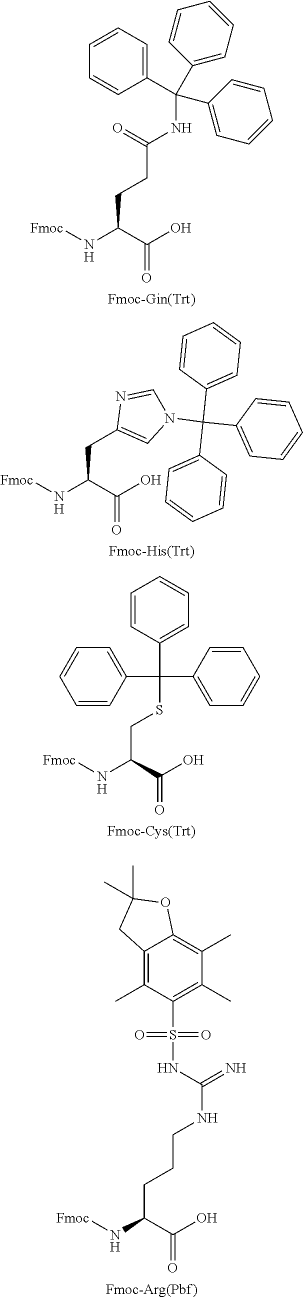

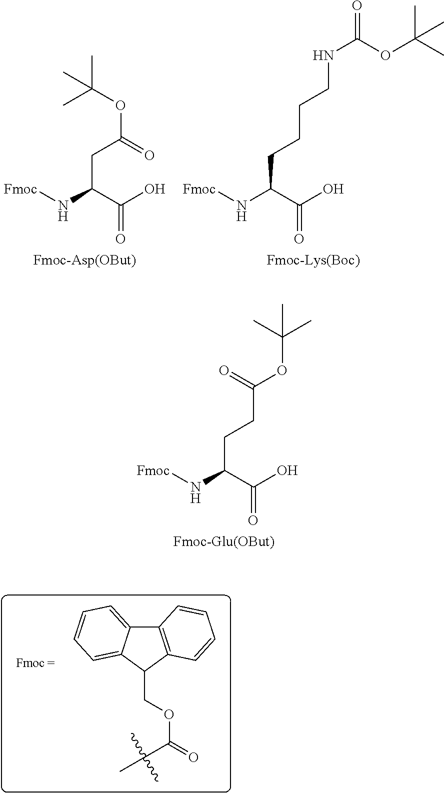

The peptidyl-resin precursors for their respective peptides may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 h at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH3CN/H20 for purification by preparative HPLC or used directly in the next step.

Peptides with the desired purity can be obtained by purification using preparative HPLC, for example, on a Waters Model 4000 or a Shimadzu Model LC-8A liquid chromatography. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified peptides can be confirmed by electro-spray MS analysis.

### Analytical Data

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m/z” unit designation. Compounds with exact masses greater than 1000 were often detected as double-charged or triple-charged ions. The crude material was purified via preparative LC/MS. Fractions containing the desired product were combined and dried via centrifugal evaporation.

### Analytical LC/MS Condition 1

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition 2

Column: Waters Acquity UPLC BEH C18, 2.1×50 mm, 1.7-μm particles; Mobile

Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

The following abbreviations are employed in the Examples and elsewhere herein:
Ph=phenyl
Bn=benzyl
i-Bu=iso-butyl
i-Pr=iso-propyl
Me=methyl Et=ethyl
Pr=n-propyl
Bu=n-butyl
t-Bu=tert-butyl
Trt=trityl TMS=trimethylsilyl
TIS=triisopropylsilane
Et₂O=diethyl ether
HOAc or AcOH=acetic acid
MeCN or AcCN=acetonitrile
DMF=N,N-dimethylformamide
EtOAc=ethyl acetate
THF=tetrahydrofuran
TFA=trifluoroacetic acid
TFE=α,α,α-trifluoroethanol
Et₂NH=diethylamine
NMM=N-methylmorpholine
NMP=N-methylpyrrolidone
DCM=dichloromethane
TEA=triethylamine
min.=minute(s)
h or hr=hour(s)
L=liter
mL or ml=milliliter
μL=microliter
g=gram(s)
mg=milligram(s)
mol=mole(s)
mmol=millimole(s)
meq=milliequivalent
rt or RT=room temperature
sat or sat'd=saturated
aq.=aqueous
mp=melting point
BOP reagen=benzotriazol-1-yloxy-tris-dimethylamino-phosphonium hexafluorophosphate (Castro's reagent)
PyB OP reagent=benzotriazol-1-yloxy-tripyrrolidino phosphonium hexafluorophosphate
HBTU=2-(1H-Benzotriazol-1-yl)-1,1,3,3-tetramethyluronim hexafluorophosphate
HATU=O-(7-Azabenzotriazol-1-yl)-1,1,3,3-tetramethyluronim hexafluorophosphate
HCTU=2-(6-Chloro-1-H-benzotriazol-1-yl)-1,1,3,3-tetramethyluronium hexafluorophosphate
T3P=2,4,6-tripropyl-1,3,5,2,4,6-trioxatriphosphorinane-2,4,6-trioxide
DMAP=4-(dimethylamino)pyridine
DIEA=diisopropylethylamine
Fmoc or FMOC=fluorenylmethyloxycarbonyl
Boc or BOC=tert-butyloxycarbonyl
HOBT or HOBTH₂O=1-hydroxybenzotriazole hydrate
Cl-HOBt=6-Chloro-benzotriazole
HOAT=1-hydroxy-7-azabenzotriazole
HPLC=high performance liquid chromatography
LC/MS=high performance liquid chromatography/mass spectrometry
MS or Mass Spec=mass spectrometry
NMR=nuclear magnetic resonance
Sc or SC or SQ=sub-cutaneous
IP or ip=intra-peritoneal

### General Procedures

### Prelude Method

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin =9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis.

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the fit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel remained attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the fit. The resin was then dried with nitrogen flow for 10 minutes. The resulting resin was used directly in the next step.

Symphony Method: Manipulations were performed under automation on a 12-channel Symphony peptide synthesizer (Protein Technologies) using procedures similar to the ones described for Prelude.

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale.

In a 30-ml bio-rad poly-prep chromatography column was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1,v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH3CN:H20 (2:3, v/v, 30 mL), and concentrated to dryness on speedvac or genevac EZ-2. This procedure was repeated (usually 2 times). The resulting crude solids were then dissolved in DMF or DMF/DMSO or CH 3 CN/H 2 0/formic acid. After filtration, the solution was subjected to single compound reverse-phase HPLC purification to afford the desired cyclic peptide.

### Preparation of Example 1000

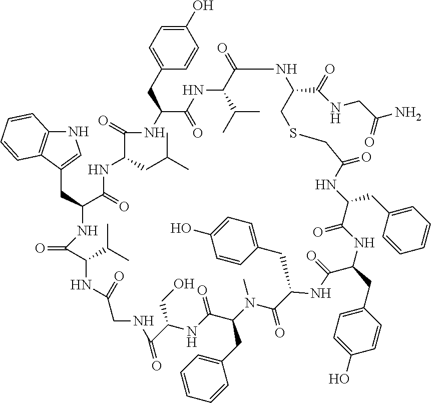 [SMILES: CC(C)C[C@@H]1NC(=O)[C@H](Cc2c[nH]c3ccccc23)NC(=O)[C@H](C(C)C)NC(=O)CNC(=O)[C@H](CO)NC(=O)[C@H](Cc2ccccc2)N(C)C(=O)[C@H](Cc2ccc(O)cc2)NC(=O)[C@H](Cc2ccc(O)cc2)NC(=O)[C@@H](Cc2ccccc2)NC(=O)CSC[C@@H](C(=O)NCC(N)=O)NC(=O)[C@H](C(C)C)NC(=O)[C@H](Cc2ccc(O)cc2)NC1=O]

A. Synthesis linear sequence Cl-Ac-dFYY-[NMe-Phe]-SGVWLYV-CG-NH₂:

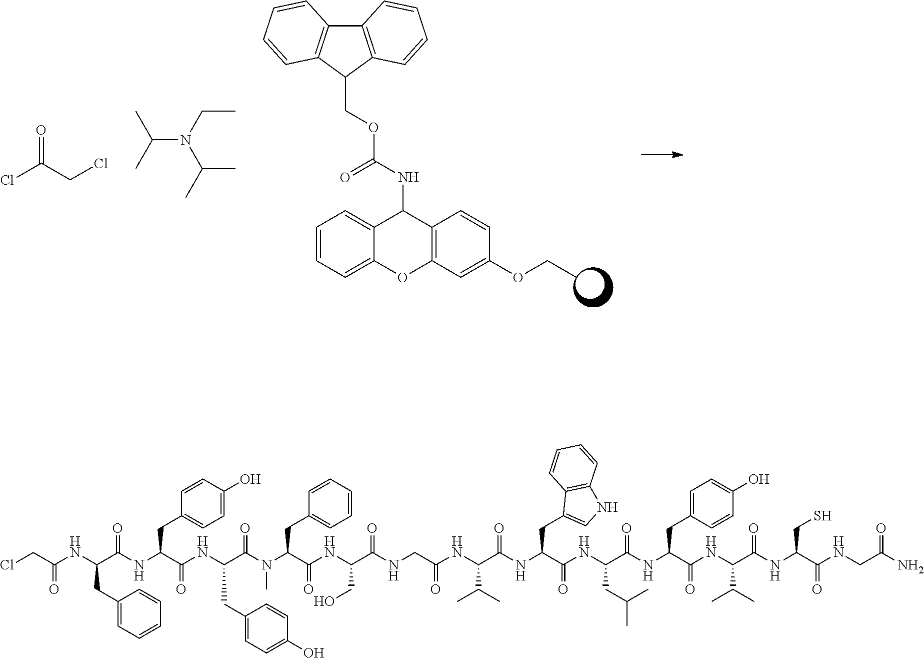

Safety precautions: 1) Wear standard PPE for chemistry (lab coat, gloves, safety glasses), and conduct as many manipulations as possible in a chemical fume hood. Dispose of waste in accordance with BMS guidelines. 2) Use caution when handling PyBOP, HCTU, HATU, DIC, or other carboxylate activators, as coupling reagents are often potent sensitizers; avoid contact with skin.

To the reactor RV5 was placed 0.1 mmol of Fmoc-Sieber Amide Resin (0.71 mmol/g, 140.5 mg) on the Prelude Synthesizer, assembled sequences (100_HC_Stnd_30 minute protocol) and (100_HC_Stnd_10×2 h protocol) for residues after a NMe amino acid . The final Fmoc group was removed with 20% Piperidine/DMF (5 mL×3 minutes×2). The resin was washed with DMF (6×5 mL×1 minute).

To a 45-mL polypropylene solid-phase reaction vessel was added Fmoc-Sieber Amide Resin (0.71 mmol/g, 140.5 mg), and the reaction vessel was placed on the Prelude peptide synthesizer. The following procedures were then performed sequentially:
“Prelude Resin-swelling procedure” was followed;
“Prelude Single-coupling procedure” was followed with Fmoc-Gly-OH;
“Prelude Single-coupling procedure” was followed with Fmoc-Val-OH;
“Prelude Single-coupling procedure” was followed with Fmoc-Tyr(tBu)-OH;
“Prelude Single-coupling procedure” was followed with Fmoc-Leu-OH;
“Prelude Single-coupling procedure” was followed with Fmoc-Trp(Boc)-OH;
“Prelude Single-coupling procedure” was followed with Fmoc-Val-OH;
“Prelude Single-coupling procedure” was followed with Fmoc-Gly-OH;
“Prelude Single-coupling procedure” was followed with Fmoc-Ser(tBu)-OH;
“Prelude Single-coupling procedure” was followed with Fmoc-NMe-Phe-OH;
“Prelude Single-coupling procedure” was followed with Fmoc-Tyr(tBu)-OH; (couple time: 2 h instead of standard 30 min);
“Prelude Single-coupling procedure” was followed with Fmoc-Tyr(tBu)-OH;
“Prelude Single-coupling procedure” was followed with Fmoc-D-Phe-OH;

To the reactor was added a solution of N,N-DIISOPROPYLETHYLAMINE (0.348 ml, 2.000 mmol) in DMF (5 ML) which was cooled in an ice bath added in 2-chloroacetyl chloride (0.161 ml, 2.000 mmol) shaken occasionally in the ice bath for 5 minutes. The reaction was shaken for 6 hours. The resin was washed with DMF (4×5 mL×1 minute), followed by DCM (4×5 mL×1 minute), and dried. Keiser test was negative.

The resin bound peptide was cleaved off the resin with (94:3:3:0.5) [v:v:v; wt] TFA/TIS/water/DTT (5 ml) for 90 min. The resin was filtered rinsed with additional cocktail (2×3 mL). The combined filtrates were evaporated. The residue was triturated with cold ether (38 mL), centrifuged, decanted, resuspended in cold ether (38 mL), centrifuged, decanted, and dried. 99724-064-20. A sample was dissolved in DMF, filtered through a 0.45-micron filter and LCMS. LCMS: (M+H+CO2)+=1739.3 use as is in the next step 99724-073.

### B. Macrocyclization:

Wear standard PPE for chemistry (lab coat, gloves, safety glasses, and safety shields), and conduct as many manipulations as possible in a chemical fume hood. Dispose of waste in accordance with safety guidelines. To the solution of crude peptide, (S)-2-((2S,5S,11S,14S,17S,20S,23R)-2-((1H-indol-3-yl)methyl)-14,23-dibenzyl- 26-chloro-17,20-bis(4-hydroxybenzyl)-11-(hydroxymethyl)-5-isopropyl-15-methyl-4,7,10,13,16,19,22,25-octaoxo-3,6,9,12,15,18,21,24-octaazahexaco sanamido)-N-((S)-1-(((S)-1-(((R)-1-((2-amino-2-oxoethyl)amino)-3-mercapto-1-oxopropan-2-yl)amino)-3-methyl-1-oxobutan-2-yl)amino)-3-(4-hydroxyphenyl)-1-oxopropan-2-yl)-4- methylpentanamide (0.169 g, 0.1 mmol) was dissolved in DMF (30 mL) was added DIEA (0.45 mL), pH 8.3. The solution was shaken 2 hours. The reaction was lyophilized, redissolved in DMF (2.2 mL), centrifuged, filtered through a 0.45-micron filter, and submitted for purification.

The crude material was purified via preparative LC/MS with the following conditions: Column: Waters XBridge C18, 19×250 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: 10-60% B over 25 minutes, then a 5-minute hold at 100% B; Flow: 20 mL/min. Fractions containing the desired product were combined and dried via centrifugal evaporation. The yield of the product was 33.1 mg (yield: 20%), and its estimated purity by LCMS analysis was 100%.

Analysis condition A: Retention time=1.54 min; ESI-MS(+) m/z [M+Na]⁺: 1674.2.

Examples 1001-1098, 1100-1124 and 1200-1206 were synthesized following similar procedures described for Example 1000.

### Preparation of Example 1001

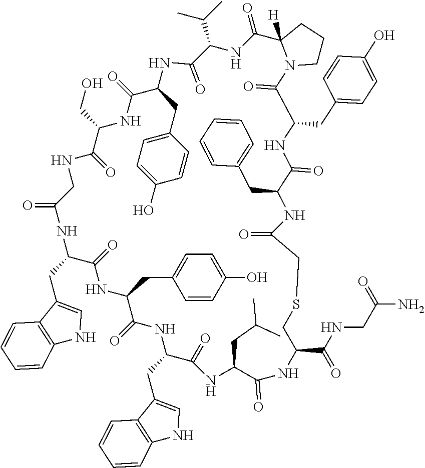 [SMILES: CC(C)C[C@@H]1NC(=O)[C@H](Cc2c[nH]c3ccccc23)NC(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](Cc2ccccc2)NC(=O)CSC[C@@H](C(=O)NCC(N)=O)NC1=O]

Example 1001 was prepared on a 100 μmol scale. The yield of the product was 57.6 mg, and its estimated purity by LCMS analysis was 97.3%. Analysis condition 1: Retention time =1.44 min; ESI-MS(+) m/z [M+H]⁺: 1697.9.

### Preparation of Example 1002

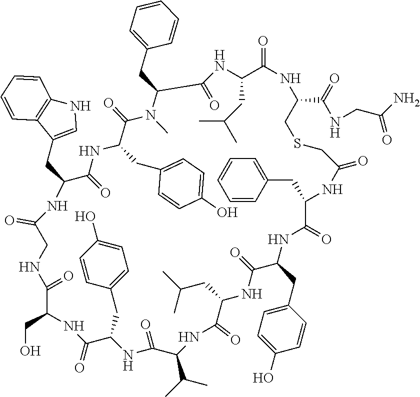 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1002 was prepared on a 100 μmol scale. The yield of the product was 62.2 mg, and its estimated purity by LCMS analysis was 100%. Analysis condition 1: Retention time=1.5 min; ESI-MS(+) m/z [M+H]⁺: 1688.9.

### Preparation of Example 1003

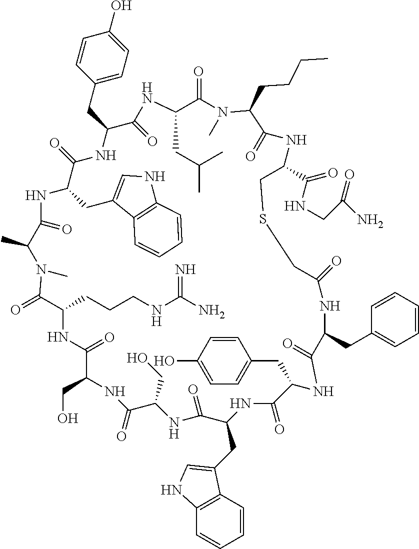 [SMILES: CCCC[C@H]1C(=O)N[C@H](C(=O)NCC(N)=O)CSCC(=O)N[C@@H](Cc2ccccc2)C(=O)N[C@@H](Cc2ccc(O)cc2)C(=O)N[C@@H](Cc2c[nH]c3ccccc23)C(=O)N[C@@H](CO)C(=O)N[C@@H](CO)C(=O)N[C@@H](CCCNC(=N)N)C(=O)N(C)[C@@H](C)C(=O)N[C@@H](Cc2c[nH]c3ccccc23)C(=O)N[C@@H](Cc2ccc(O)cc2)C(=O)N[C@@H](CC(C)C)C(=O)N1C]

Example 1003 was prepared on a 100 μmol scale. The yield of the product was 37.4 mg, and its estimated purity by LCMS analysis was 95.6%. Analysis condition 2: Retention time=1.4 min; ESI-MS(+) m/z [M+H]⁺: 1718.9.

### Preparation of Example 1004

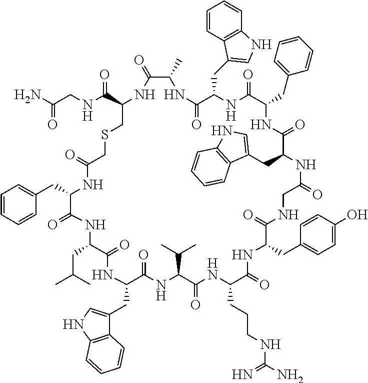 [SMILES: CC(C)C[C@@H]1NC(=O)[C@H](Cc2ccccc2)NC(=O)CSC[C@@H](C(=O)NCC(N)=O)NC(=O)[C@H](C)NC(=O)[C@H](Cc2c[nH]c3ccccc23)NC(=O)[C@H](Cc2ccccc2)NC(=O)[C@H](Cc2c[nH]c3ccccc23)NC(=O)CNC(=O)[C@H](Cc2ccc(O)cc2)NC(=O)[C@H](CCCNC(=N)N)NC(=O)[C@H](C(C)C)NC(=O)[C@H](Cc2c[nH]c3ccccc23)NC1=O]

Example 1004 was prepared on a 50 μmol scale. The yield of the product was 22.5 mg, and its estimated purity by LCMS analysis was 94%. Analysis condition 2: Retention time=1.61 min; ESI-MS(+) m/z [M+H]⁺: 1731.2.

### Preparation of Example 1005

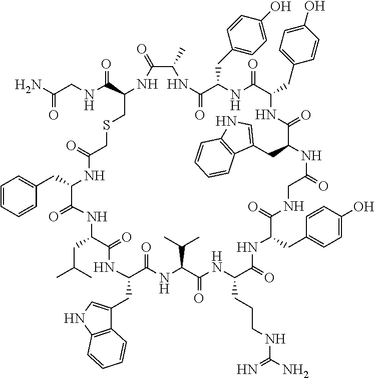 [SMILES: CC(C)C[C@@H]1NC(=O)[C@H](Cc2ccccc2)NC(=O)CSC[C@@H](C(=O)NCC(N)=O)NC(=O)[C@H](C)NC(=O)[C@H](Cc2ccc(O)cc2)NC(=O)[C@H](Cc2ccc(O)cc2)NC(=O)[C@H](Cc2c[nH]c3ccccc23)NC(=O)CNC(=O)[C@H](Cc2ccc(O)cc2)NC(=O)[C@H](CCCNC(=N)N)NC(=O)[C@H](C(C)C)NC(=O)[C@H](Cc2c[nH]c3ccccc23)NC1=O]

Example 1005 was prepared on a 50 mmol scale. The yield of the product was 7.7 mg, and its estimated purity by LCMS analysis was 95.6%. Analysis condition 2: Retention time=1.4 min; ESI-MS(+) m/z [M+H]⁺: 1723.4.

### Preparation of Example 1006

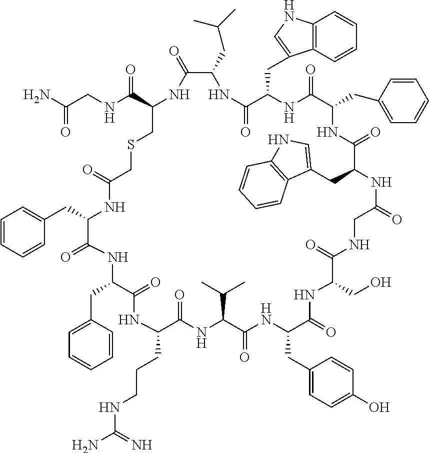 [SMILES: CC(C)C[C@@H]1NC(=O)[C@H](Cc2c[nH]c3ccccc23)NC(=O)[C@H](Cc2ccccc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CCCNC(=N)N)NC(=O)[C@H](Cc2ccccc2)NC(=O)[C@H](Cc2ccccc2)NC(=O)CSC[C@@H](C(=O)NCC(N)=O)NC1=O]

Example 1006 was prepared on a 50 μmol scale. The yield of the product was 11.2 mg, and its estimated purity by LCMS analysis was 98.2%. Analysis condition 2: Retention time=1.62 min; ESI-MS(+) m/z [M+H]⁺: 1707.5.

### Preparation of Example 1007

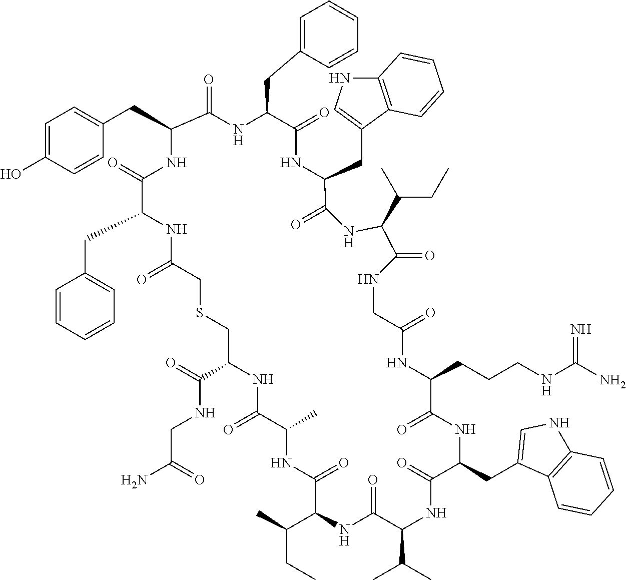 [SMILES: CCC(C)[C@@H]1NC(=O)[C@H](Cc2c[nH]c3ccccc23)NC(=O)[C@H](Cc2ccccc2)NC(=O)[C@H](Cc2ccc(O)cc2)NC(=O)[C@@H](Cc2ccccc2)NC(=O)CSC[C@@H](C(=O)NCC(N)=O)NC(=O)[C@H](C)NC(=O)[C@H]([C@H](C)CC)NC(=O)[C@H](C(C)C)NC(=O)[C@H](Cc2c[nH]c3ccccc23)NC(=O)[C@H](CCCNC(=N)N)NC(=O)CNC1=O]

Example 1007 was prepared on a 50 μmol scale. The yield of the product was 2.2 mg, and its estimated purity by LCMS analysis was 91.7%. Analysis condition 1: Retention time=1.55 min; ESI-MS(+) m/z [M+H]⁺: 1680.1.

### Preparation of Example 1008

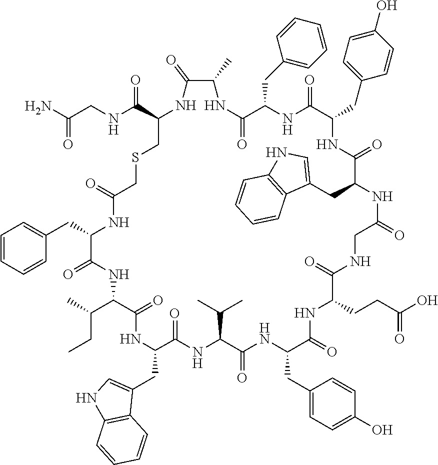 [SMILES: CC[C@H](C)[C@@H]1NC(=O)[C@H](Cc2ccccc2)NC(=O)CSC[C@@H](C(=O)NCC(N)=O)NC(=O)[C@H](C)NC(=O)[C@H](Cc2ccccc2)NC(=O)[C@H](Cc2ccc(O)cc2)NC(=O)[C@H](Cc2c[nH]c3ccccc23)NC(=O)CNC(=O)[C@H](CCC(=O)O)NC(=O)[C@H](Cc2ccc(O)cc2)NC(=O)[C@H](C(C)C)NC(=O)[C@H](Cc2c[nH]c3ccccc23)NC1=O]

Example 1008 was prepared on a 50 μmol scale. The yield of the product was 2.5 mg, and its estimated purity by LCMS analysis was 96.8%. Analysis condition 2: Retention time=1.49 min; ESI-MS(+) m/z [M+H]⁺: 1702.4.

### Preparation of Example 1009

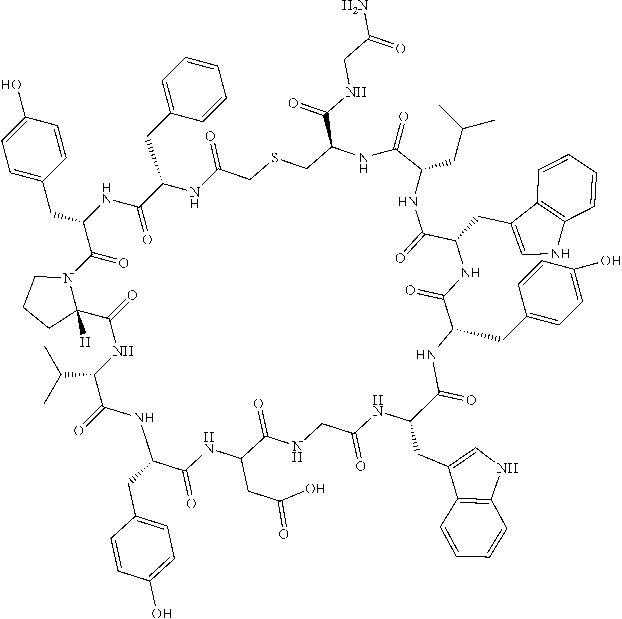 [SMILES: CC(C)C[C@@H]1NC(=O)[C@H](Cc2c[nH]c3ccccc23)NC(=O)[C@H](Cc2ccc(O)cc2)NC(=O)[C@H](Cc2c[nH]c3ccccc23)NC(=O)CNC(=O)C(CC(=O)O)NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](Cc2ccccc2)NC(=O)CSC[C@@H](C(=O)NCC(N)=O)NC1=O]

Example 1009 was prepared on a 50 μmol scale. The yield of the product was 7.6 mg, and its estimated purity by LCMS analysis was 90.3%. Analysis condition 2: Retention time=1.4 min; ESI-MS(+) m/z [M+2H]²⁺: 855.

### Preparation of Example 1010

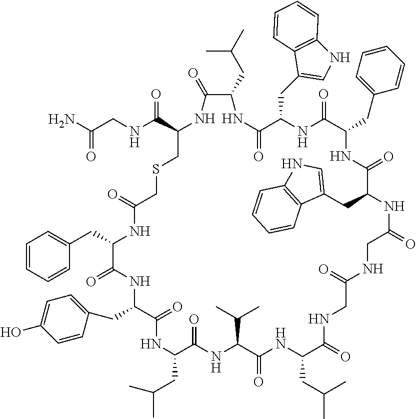 [SMILES: CC(C)C[C@@H]1NC(=O)[C@H](Cc2c[nH]c3ccccc23)NC(=O)[C@H](Cc2ccccc2)NC(=O)[C@H](Cc2c[nH]c3ccccc23)NC(=O)CNC(=O)CNC(=O)[C@H](CC(C)C)NC(=O)[C@H](C(C)C)NC(=O)[C@H](CC(C)C)NC(=O)[C@H](Cc2ccc(O)cc2)NC(=O)[C@H](Cc2ccccc2)NC(=O)CSC[C@@H](C(=O)NCC(N)=O)NC1=O]

Example 1010 was prepared on a 50 μmol scale. The yield of the product was 10.9 mg, and its estimated purity by LCMS analysis was 86%. Analysis condition 1: Retention time=1.78 min; ESI-MS(+) m/z [M+H]⁺: 1600.2.

### Preparation of Example 1011

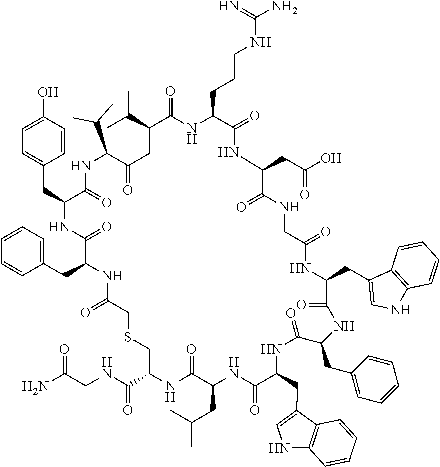 [SMILES: CC(C)C[C@@H]1NC(=O)[C@H](Cc2c[nH]c3ccccc23)NC(=O)[C@H](Cc2ccccc2)NC(=O)[C@H](Cc2c[nH]c3ccccc23)NC(=O)CNC(=O)[C@H](CC(=O)O)NC(=O)[C@H](CCCNC(=N)N)NC(=O)[C@H](C(C)C)CC(=O)[C@H](C(C)C)NC(=O)[C@H](Cc2ccc(O)cc2)NC(=O)[C@H](Cc2ccccc2)NC(=O)CSC[C@@H](C(=O)NCC(N)=O)NC1=O]

Example 1011 was prepared on a 50 μmol scale. The yield of the product was 18.3 mg, and its estimated purity by LCMS analysis was 98.5%. Analysis condition 2: Retention time=1.57 min; ESI-MS(+) m/z [M+H]⁺: 1687.5.

### Preparation of Example 1012

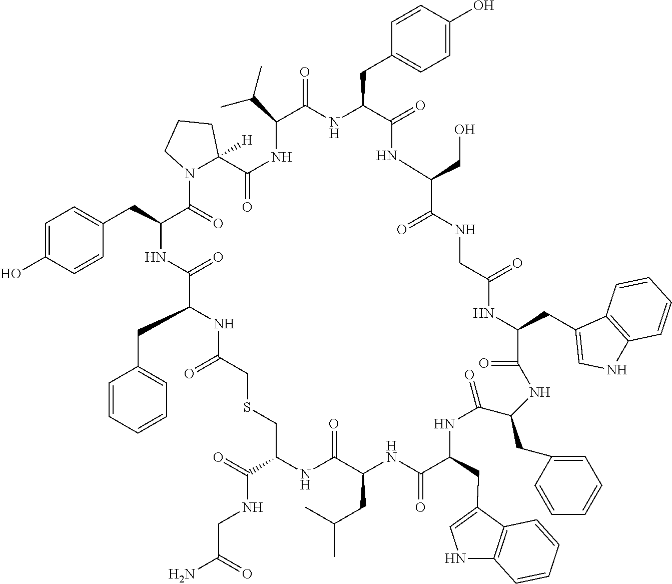 [SMILES: CC(C)C[C@@H]1NC(=O)[C@H](Cc2c[nH]c3ccccc23)NC(=O)[C@H](Cc2ccccc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](Cc2ccccc2)NC(=O)CSC[C@@H](C(=O)NCC(N)=O)NC1=O]

Example 1012 was prepared on a 50 μmol scale. The yield of the product was 21.8 mg, and its estimated purity by LCMS analysis was 96.6%. Analysis condition 2: Retention time=1.67 min; ESI-MS(+) m/z [M+H]⁺: 1663.4.

### Preparation of Example 1013

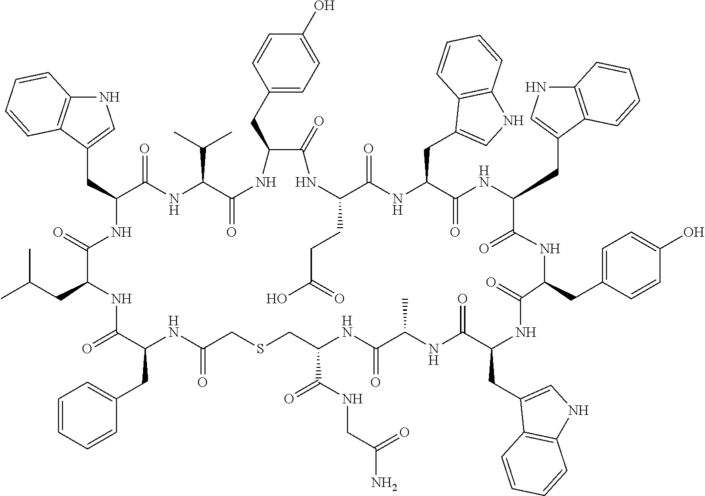 [SMILES: CC(C)C[C@@H]1NC(=O)[C@H](Cc2ccccc2)NC(=O)CSC[C@@H](C(=O)NCC(N)=O)NC(=O)[C@H](C)NC(=O)[C@H](Cc2c[nH]c3ccccc23)NC(=O)[C@H](Cc2ccc(O)cc2)NC(=O)[C@H](Cc2c[nH]c3ccccc23)NC(=O)[C@H](Cc2c[nH]c3ccccc23)NC(=O)[C@H](CCC(=O)O)NC(=O)[C@H](Cc2ccc(O)cc2)NC(=O)[C@H](C(C)C)NC(=O)[C@H](Cc2c[nH]c3ccccc23)NC1=O]

Example 1013 was prepared on a 50 μmol scale. The yield of the product was 1.3 mg, and its estimated purity by LCMS analysis was 97.4%. Analysis condition 1: Retention time=1.65 min; ESI-MS(+) m/z [M+H]⁺: 1849.1.

### Preparation of Example 1014

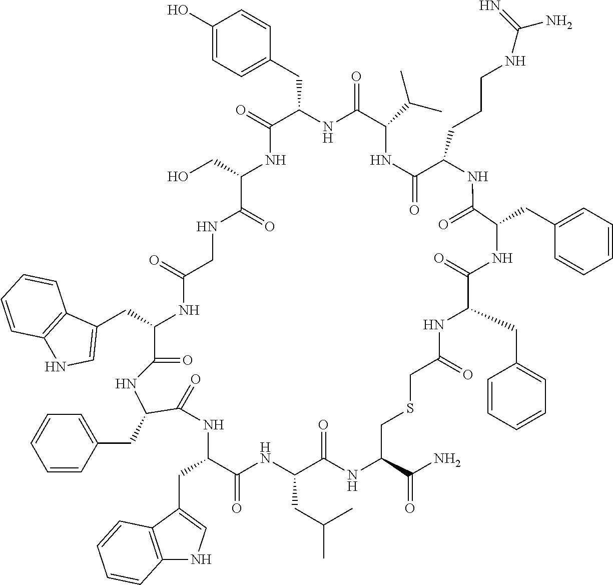 [SMILES: CC(C)C[C@@H]1NC(=O)[C@H](Cc2c[nH]c3ccccc23)NC(=O)[C@H](Cc2ccccc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CCCNC(=N)N)NC(=O)[C@H](Cc2ccccc2)NC(=O)[C@H](Cc2ccccc2)NC(=O)CSC[C@@H](C(N)=O)NC1=O]

Example 1014 was prepared on a 50 μmol scale. The yield of the product was 9.2 mg, and its estimated purity by LCMS analysis was 96.9%. Analysis condition 2: Retention time=1.54 min; ESI-MS(+) m/z [M+H]⁺: 1649.9.

### Preparation of Example 1015

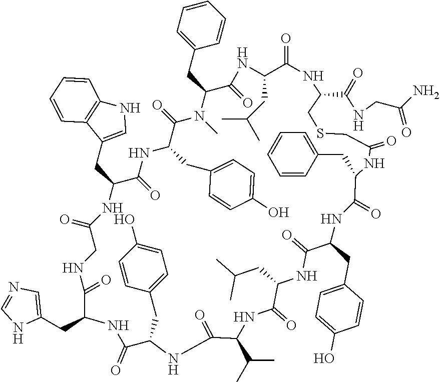 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](Cc2cnc[nH]2)NC(=O)[C@H](Cc2ccc(O)cc2)NC(=O)[C@H](C(C)C)NC1=O]

Example 1015 was prepared on a 40 μmol scale. The yield of the product was 7.5 mg, and its estimated purity by LCMS analysis was 96.4%. Analysis condition 1: Retention time=1.47 min; ESI-MS(+) m/z [M+H]⁺: 1721.

### Preparation of Example 1016

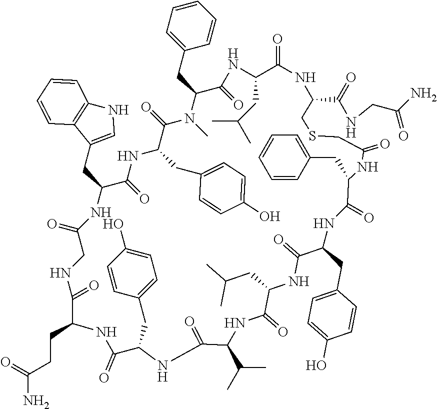 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CCC(N)=O)NC(=O)[C@H](Cc2ccc(O)cc2)NC(=O)[C@H](C(C)C)NC1=O]

Example 1016 was prepared on a 50 μmol scale. The yield of the product was 6.5 mg, and its estimated purity by LCMS analysis was 87.8%. Analysis condition 1: Retention time=1.46 min; ESI-MS(+) m/z [M+2H]²⁺: 857.2.

### Preparation of Example 1017

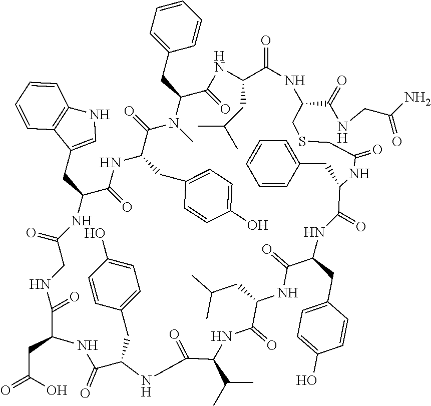 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CC(=O)O)NC(=O)[C@H](Cc2ccc(O)cc2)NC(=O)[C@H](C(C)C)NC1=O]

Example 1017 was prepared on a 40 μmol scale. The yield of the product was 9.6 mg, and its estimated purity by LCMS analysis was 86.9%. Analysis condition 1: Retention time=1.29, 1.38 min; ESI-MS(+) m/z [M+H]⁺: 1717.1.

### Preparation of Example 1018

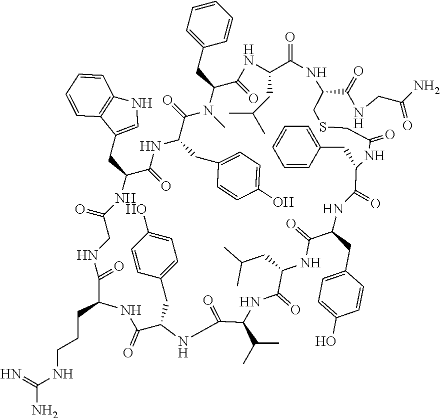 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CCCNC(=N)N)NC(=O)[C@H](Cc2ccc(O)cc2)NC(=O)[C@H](C(C)C)NC1=O]

Example 1018 was prepared on a 50 μmol scale. The yield of the product was 7.3 mg, and its estimated purity by LCMS analysis was 96.4%. Analysis condition 1: Retention time =1.45 min; ESI-MS(+) m/z [M+H]⁺: 1741.2.

### Preparation of Example 1019

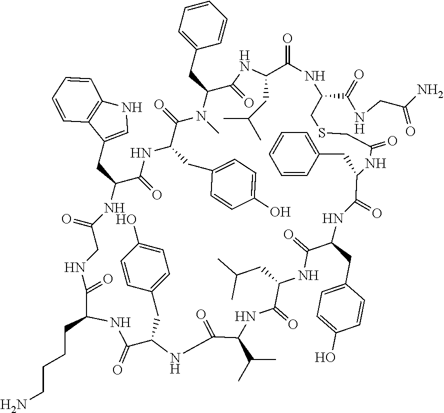 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CCCCN)NC(=O)[C@H](Cc2ccc(O)cc2)NC(=O)[C@H](C(C)C)NC1=O]

Example 1019 was prepared on a 50 μmol scale. The yield of the product was 9.1 mg, and its estimated purity by LCMS analysis was 93%. Analysis condition 2: Retention time=1.49 min; ESI-MS(+) m/z [M+H]⁺: 1713.

### Preparation of Example 1020

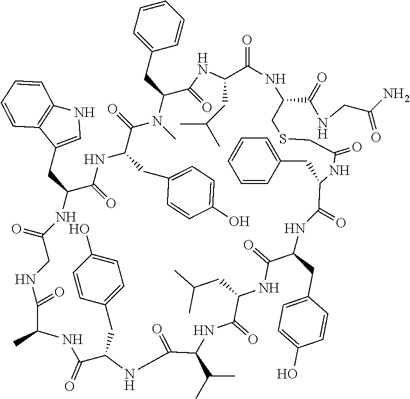 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](C)NC(=O)[C@H](Cc2ccc(O)cc2)NC(=O)[C@H](C(C)C)NC1=O]

Example 1020 was prepared on a 50 μmol scale. The yield of the product was 4.8 mg, and its estimated purity by LCMS analysis was 89.8%. Analysis condition 1: Retention time=1.54 min; ESI-MS(+) m/z [M+2H]²⁺: 828.

### Preparation of Example 1021

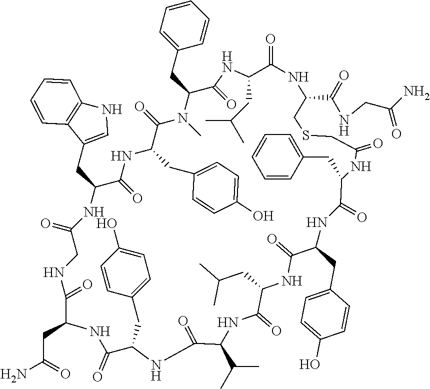 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CC(N)=O)NC(=O)[C@H](Cc2ccc(O)cc2)NC(=O)[C@H](C(C)C)NC1=O]

Example 1021 was prepared on a 30 μmol scale. The yield of the product was 8.4 mg, and its estimated purity by LCMS analysis was 95.5%. Analysis condition 2: Retention time=1.42 min; ESI-MS(+) m/z [M+H]⁺: 1698.8.

### Preparation of Example 1022

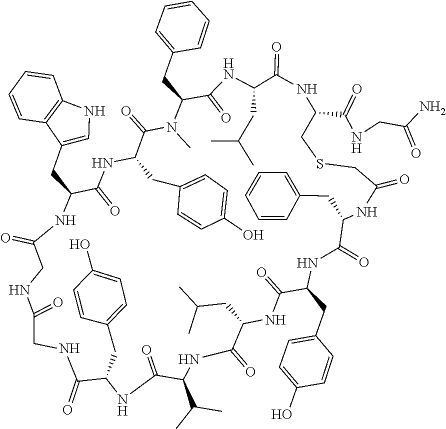 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)CNC(=O)[C@H](Cc2ccc(O)cc2)NC(=O)[C@H](C(C)C)NC1=O]

Example 1022 was prepared on a 50 μmol scale. The yield of the product was 4.7 mg, and its estimated purity by LCMS analysis was 98.9%. Analysis condition 1: Retention time=1.4 min; ESI-MS(+) m/z [M+H]⁺: 1658.

### Preparation of Example 1023

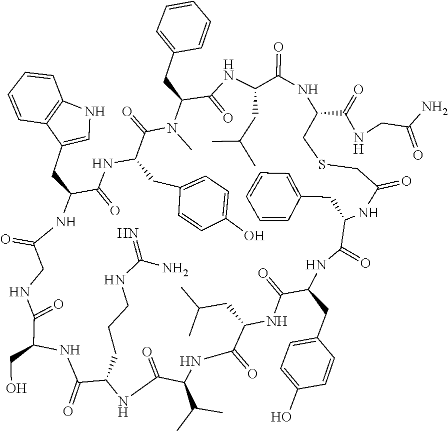 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](CCCNC(=N)N)NC(=O)[C@H](C(C)C)NC1=O]

Example 1023 was prepared on a 40 μmol scale. The yield of the product was 9.2 mg, and its estimated purity by LCMS analysis was 95.5%. Analysis condition 1: Retention time=1.39 min; ESI-MS(+) m/z [M+H]⁺: 1664.1.

### Preparation of Example 1024

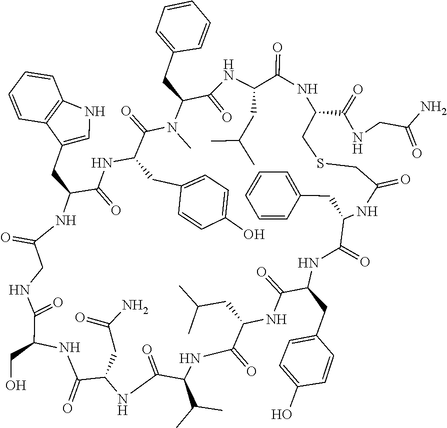 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](CC(N)=O)NC(=O)[C@H](C(C)C)NC1=O]

Example 1024 was prepared on a 40 μmol scale. The yield of the product was 7 mg, and its estimated purity by LCMS analysis was 95.6%. Analysis condition 2: Retention time=1.36 min; ESI-MS(+) m/z [M+H]⁺: 1621.6.

### Preparation of Example 1025

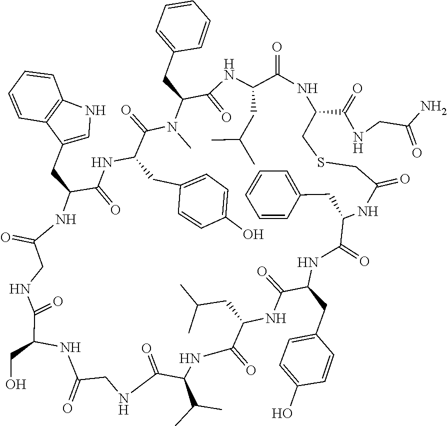 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)CNC(=O)[C@H](C(C)C)NC1=O]

Example 1025 was prepared on a 40 μmol scale. The yield of the product was 13.7 mg, and its estimated purity by LCMS analysis was 95.2%. Analysis condition 2: Retention time=1.39 min; ESI-MS(+) m/z [M+H]⁺: 1565.1.

### Preparation of Example 1026

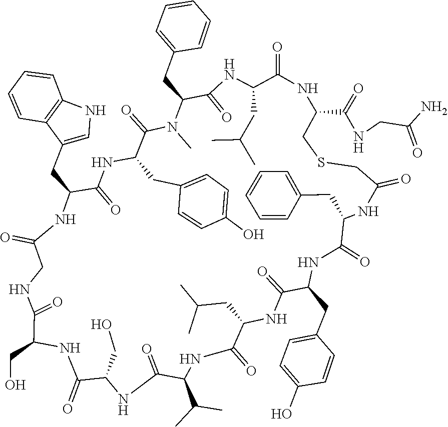 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](CO)NC(=O)[C@H](C(C)C)NC1=O]

Example 1026 was prepared on a 40 μmol scale. The yield of the product was 12.5 mg, and its estimated purity by LCMS analysis was 95.1%. Analysis condition 2: Retention time=1.42 min; ESI-MS(+) m/z [M+H]⁺: 1595.1.

### Preparation of Example 1027

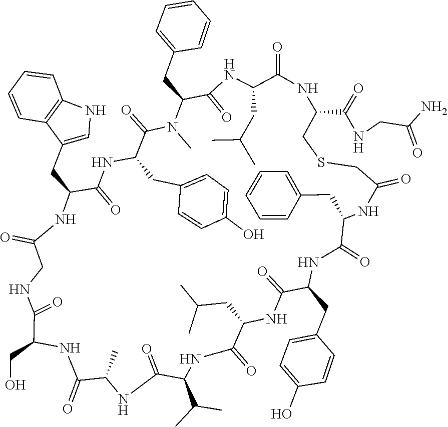 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](C)NC(=O)[C@H](C(C)C)NC1=O]

Example 1027 was prepared on a 40 μmol scale. The yield of the product was 8 mg, and its estimated purity by LCMS analysis was 88.9%. Analysis condition 1: Retention time=1.44 min; ESI-MS(+) m/z [M+H]⁺: 1597.

### Preparation of Example 1028

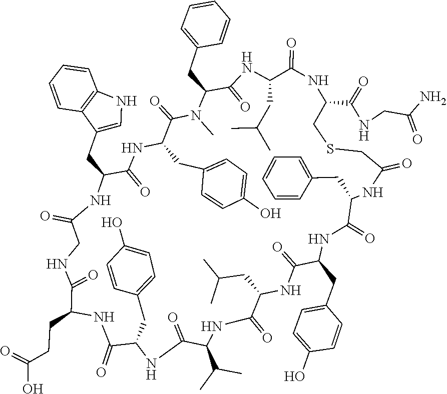 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CCC(=O)O)NC(=O)[C@H](Cc2ccc(O)cc2)NC(=O)[C@H](C(C)C)NC1=O]

Example 1028 was prepared on a 40 μmol scale. The yield of the product was 4.9 mg, and its estimated purity by LCMS analysis was 95.6%. Analysis condition 2: Retention time=1.49 min; ESI-MS(+) m/z [M+H]⁺: 1712.9.

### Preparation of Example 1029

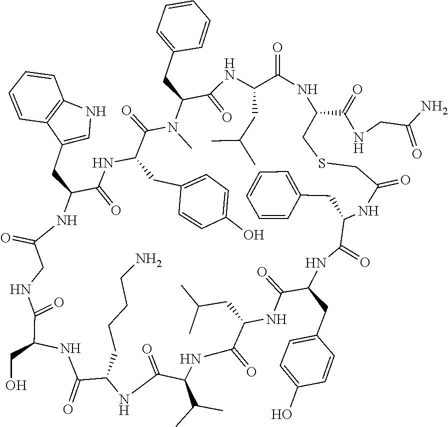 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](CCCCN)NC(=O)[C@H](C(C)C)NC1=O]

Example 1029 was prepared on a 40 μmol scale. The yield of the product was 6.8 mg, and its estimated purity by LCMS analysis was 97%. Analysis condition 2: Retention time=1.36 min; ESI-MS(+) m/z [M+H]⁺: 1636.

### Preparation of Example 1030

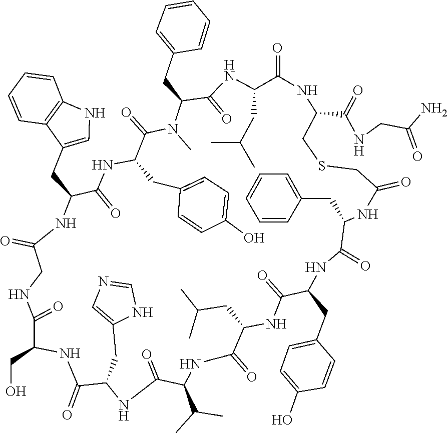 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2cnc[nH]2)NC(=O)[C@H](C(C)C)NC1=O]

Example 1030 was prepared on a 40 μmol scale. The yield of the product was 7.5 mg, and its estimated purity by LCMS analysis was 96.6%. Analysis condition 2: Retention time=1.37 min; ESI-MS(+) m/z [M+2H]²⁺: 822.9.

### Preparation of Example 1031

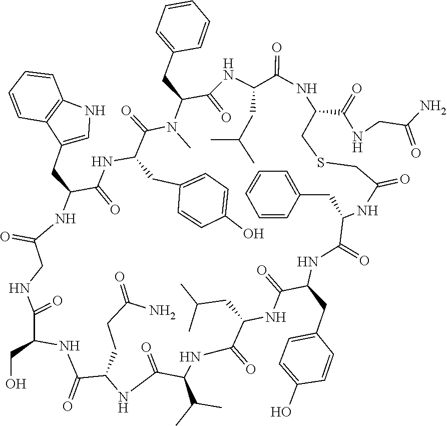 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](CCC(N)=O)NC(=O)[C@H](C(C)C)NC1=O]

Example 1031 was prepared on a 40 μmol scale. The yield of the product was 8.5 mg, and its estimated purity by LCMS analysis was 96.7%. Analysis condition 2: Retention time=1.4 min; ESI-MS(+) m/z [M+H]⁺: 1635.8.

### Preparation of Example 1032

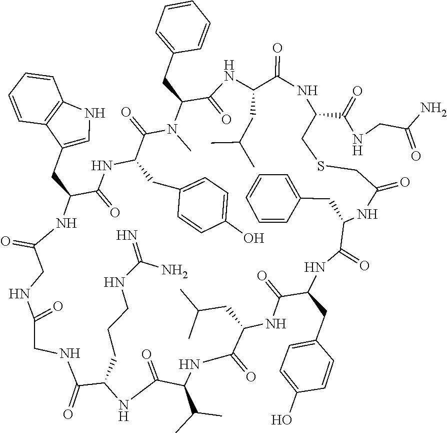 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)CNC(=O)[C@H](CCCNC(=N)N)NC(=O)[C@H](C(C)C)NC1=O]

Example 1032 was prepared on a 40 μmol scale. The yield of the product was 6 mg, and its estimated purity by LCMS analysis was 96.8%. Analysis condition 2: Retention time=1.36 min; ESI-MS(+) m/z [M+H]⁺: 1633.9.

### Preparation of Example 1033

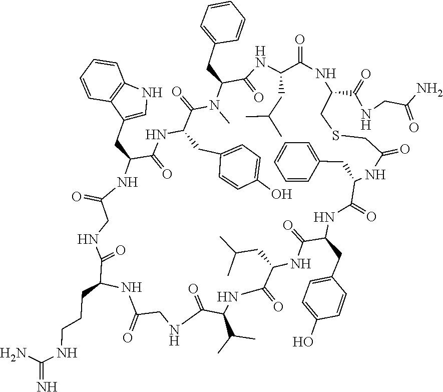 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CCCNC(=N)N)NC(=O)CNC(=O)[C@H](C(C)C)NC1=O]

Example 1033 was prepared on a 40 μmol scale. The yield of the product was 9.8 mg, and its estimated purity by LCMS analysis was 89.4%. Analysis condition 1: Retention time=1.36 min; ESI-MS(+) m/z [M+H]⁺: 1634.2.

### Preparation of Example 1034

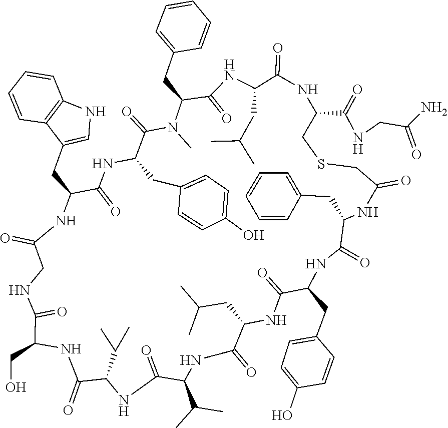 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](C(C)C)NC(=O)[C@H](C(C)C)NC1=O]

Example 1034 was prepared on a 20 μmol scale. The yield of the product was 2.6 mg, and its estimated purity by LCMS analysis was 96.8%. Analysis condition 1: Retention time=1.57 min; ESI-MS(+) m/z [M+H]⁺: 1624.8.

### Preparation of Example 1035

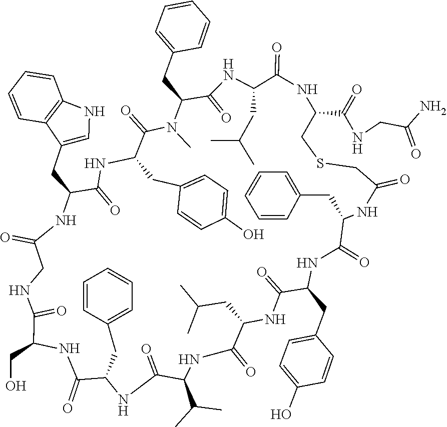 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccccc2)NC(=O)[C@H](C(C)C)NC1=O]

Example 1035 was prepared on a 40 μmol scale. The yield of the product was 7.9 mg, and its estimated purity by

LCMS analysis was 95.2%. Analysis condition 2: Retention time=1.65 min; ESI-MS(+) m/z [M+H]⁺: 1655.2.

### Preparation of Example 1036

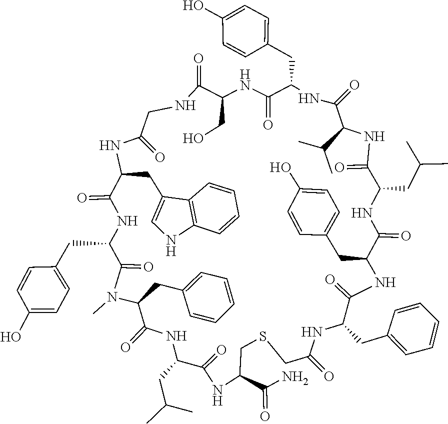 [SMILES: CC(C)C[C@@H]1NC(=O)[C@H](Cc2ccc(O)cc2)NC(=O)[C@H](Cc2ccccc2)NC(=O)CSC[C@@H](C(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1036 was prepared on a 100 μmol scale. The yield of the product was 40 mg, and its estimated purity by LCMS analysis was 93%. Analysis condition 2: Retention time=1.61 min; ESI-MS(+) m/z [M+H]⁺: 1613.9.

### Preparation of Example 1037

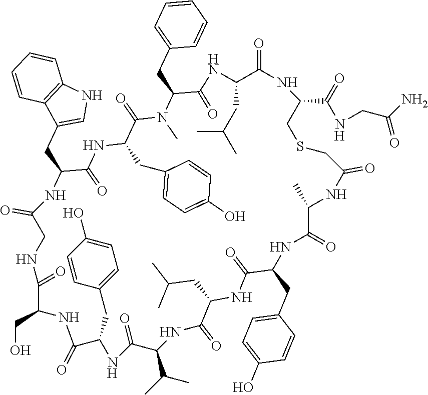

Example 1037 was prepared on a 100 μmol scale. The yield of the product was 16.5 mg, and its estimated purity by LCMS analysis was 100%. Analysis condition 1: Retention time=1.4 min; ESI-MS(+) m/z [M+H]⁺: 1594.6.

### Preparation of Example 1038

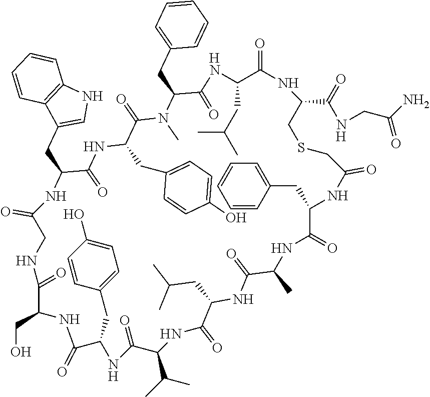

Example 1038 was prepared on a 100 μmol scale. The yield of the product was 13.4 mg, and its estimated purity by LCMS analysis was 99.3%. Analysis condition 1: Retention time=1.57 min; ESI-MS(+) m/z [M+2H]²⁺: 790.1.

### Preparation of Example 1039

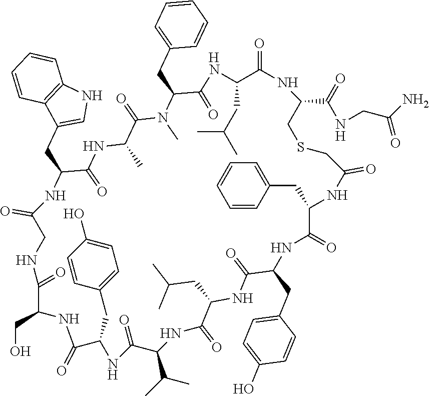

Example 1039 was prepared on a 50 μmol scale. The yield of the product was 25.3 mg, and its estimated purity by LCMS analysis was 97.9%. Analysis condition 2: Retention time=1.52 min; ESI-MS(+) m/z [M+H]⁺: 1578.9.

### Preparation of Example 1040

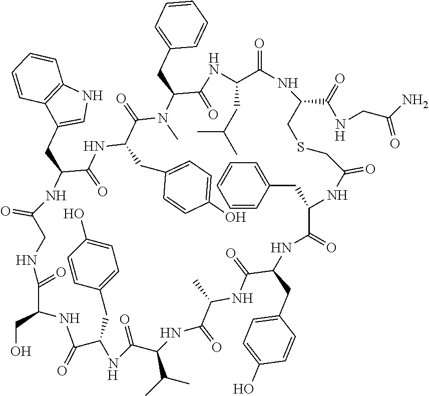

Example 1040 was prepared on a 100 μmol scale. The yield of the product was 29 mg, and its estimated purity by LCMS analysis was 91.9%. Analysis condition 1: Retention time=1.45 min; ESI-MS(+) m/z [M+H]⁺: 1645.6.

### Preparation of Example 1041

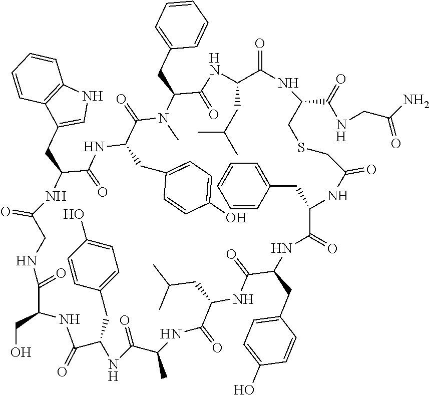

Example 1041 was prepared on a 100 μmol scale. The yield of the product was 33.2 mg, and its estimated purity by LCMS analysis was 98.1%. Analysis condition 1: Retention time=1.54 min; ESI-MS(+) m/z [M+H]⁺: 1642.8.

### Preparation of Example 1042

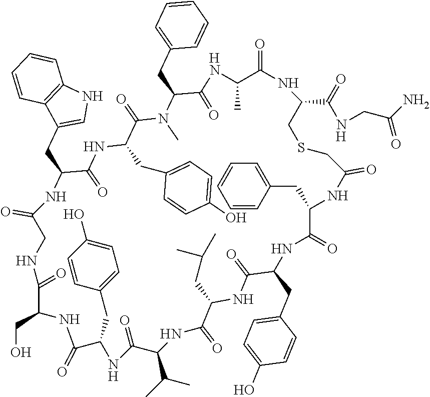

Example 1042 was prepared on a 100 μmol scale. The yield of the product was 19.3 mg, and its estimated purity by LCMS analysis was 100%. Analysis condition 1: Retention time=1.43 min; ESI-MS(+) m/z [M+2H]²⁺: 815.

### Preparation of Example 1043

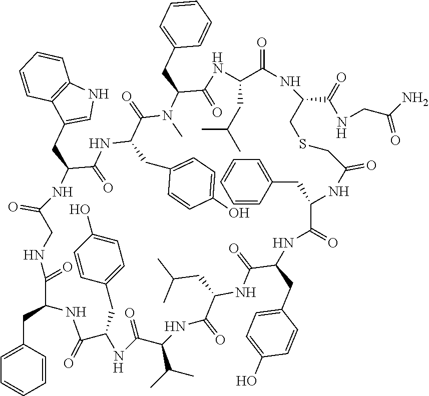

Example 1043 was prepared on a 40 μmol scale. The yield of the product was 7.9 mg, and its estimated purity by LCMS analysis was 99.1%. Analysis condition 1: Retention time=1.79 min; ESI-MS(+) m/z [M+H]⁺: 1731.1.

### Preparation of Example 1044

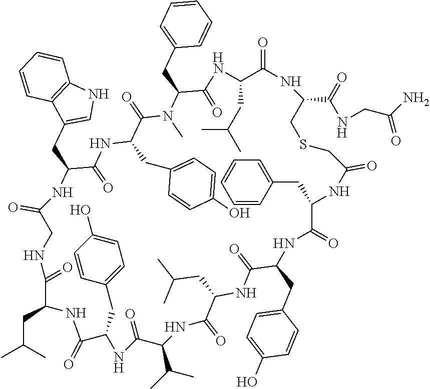

Example 1044 was prepared on a 0.1 mmol scale. The yield of the product was 6.3 mg, and its estimated purity by LCMS analysis was 97.8%. Analysis condition 2: Retention time=1.71 min; ESI-MS(+) m/z [M+H]⁺: 1697.

### Preparation of Example 1045

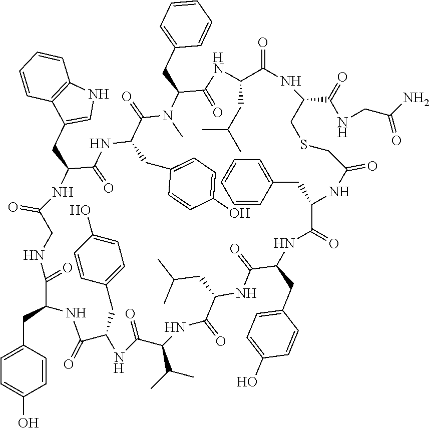

Example 1045 was prepared on a 40 μmol scale. The yield of the product was 6.7 mg, and its estimated purity by LCMS analysis was 99.3%. Analysis condition 1: Retention time=1.57 min; ESI-MS(+) m/z [M+H]⁺: 1764.9.

### Preparation of Example 1046

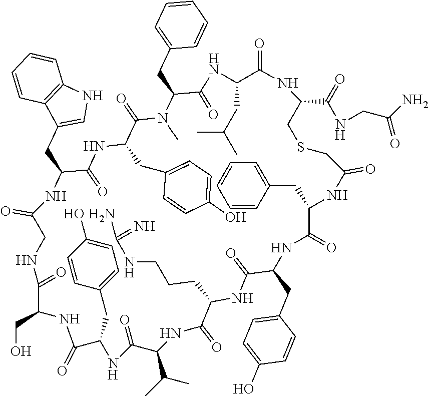

Example 1046 was prepared on a 50 μmol scale. The yield of the product was 15.9 mg, and its estimated purity by LCMS analysis was 99.2%. Analysis condition 1: Retention time=1.3 min; ESI-MS(+) m/z [M+2H]²⁺: 857.1.

### Preparation of Example 1047

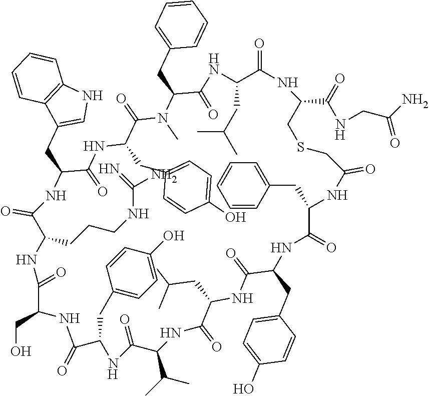

Example 1047 was prepared on a 50 μmol scale. The yield of the product was 15.4 mg, and its estimated purity by LCMS analysis was 100%. Analysis condition 1: Retention time=1.29 min; ESI-MS(+) m/z [M+2H]²⁺: 885.1.

### Preparation of Example 1048

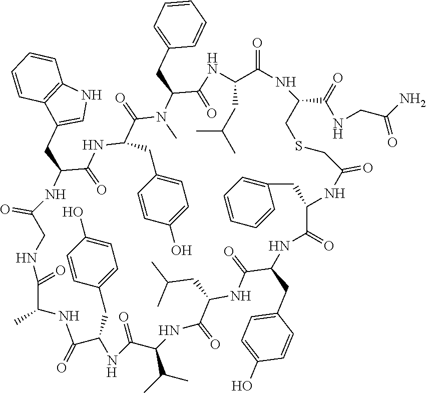

Example 1048 was prepared on a 50 mmol scale. The yield of the product was 17.6 mg, and its estimated purity by LCMS analysis was 99.4%. Analysis condition 2: Retention time=1.53 min; ESI-MS(+) m/z [M+H]⁺: 1654.9.

### Preparation of Example 1049

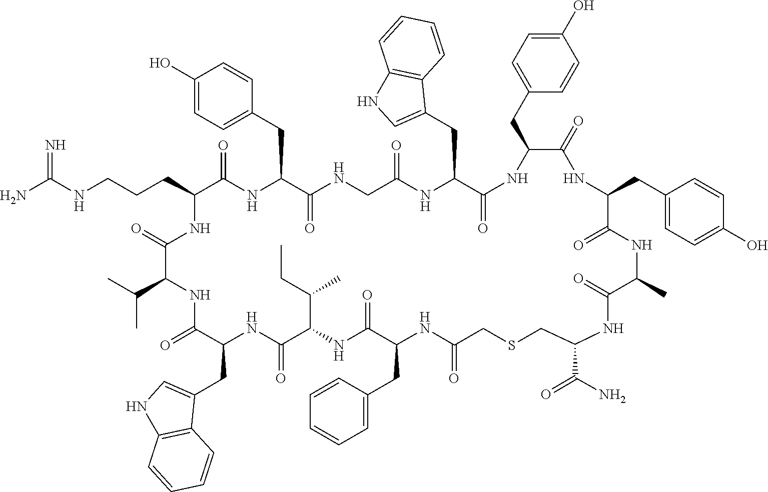 [SMILES: CC[C@H](C)[C@@H]1NC(=O)[C@H](Cc2ccccc2)NC(=O)CSC[C@@H](C(N)=O)NC(=O)[C@H](C)NC(=O)[C@H](Cc2ccc(O)cc2)NC(=O)[C@H](Cc2ccc(O)cc2)NC(=O)[C@H](Cc2c[nH]c3ccccc23)NC(=O)CNC(=O)[C@H](Cc2ccc(O)cc2)NC(=O)[C@H](CCCNC(=N)N)NC(=O)[C@H](C(C)C)NC(=O)[C@H](Cc2c[nH]c3ccccc23)NC1=O]

Example 1049 was prepared on a 50 μmol scale. The yield of the product was 4.9 mg, and its estimated purity by LCMS analysis was 95.7%. Analysis condition 2: Retention time=1.31 min; ESI-MS(+) m/z [M+H]⁺: 1665.8.

### Preparation of Example 1050

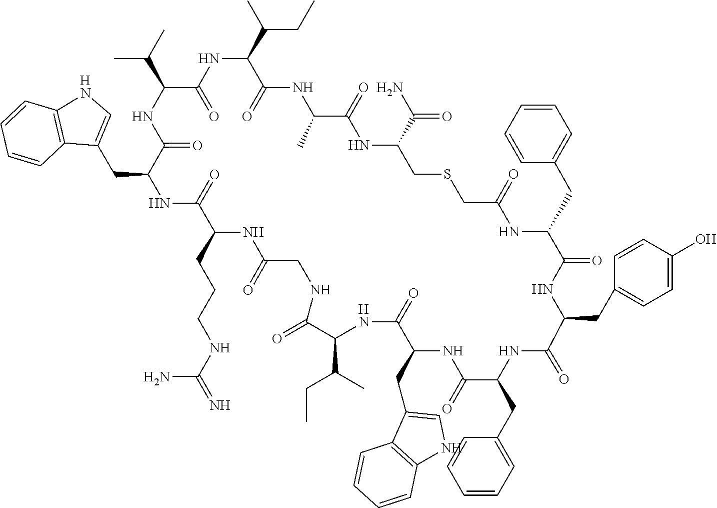

Example 1050 was prepared on a 50 μmol scale. The yield of the product was 3.8 mg, and its estimated purity by LCMS analysis was 97.1%. Analysis condition 2: Retention time=1.49 min; ESI-MS(+) m/z [M+H]⁺: 1599.8.

### Preparation of Example 1051

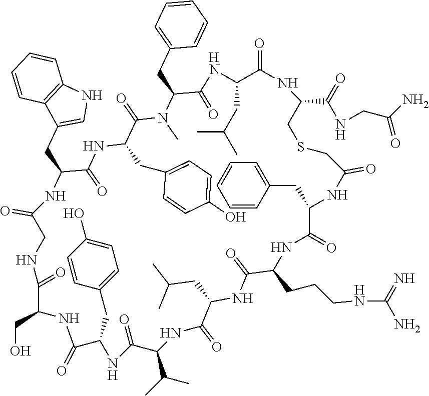

Example 1051 was prepared on a 0.1 mmol scale. The yield of the product was 28 mg, and its estimated purity by LCMS analysis was 100%. Analysis condition 1: Retention time=1.45 min; ESI-MS(+) m/z [M+H]⁺: 1664.2.

### Preparation of Example 1052

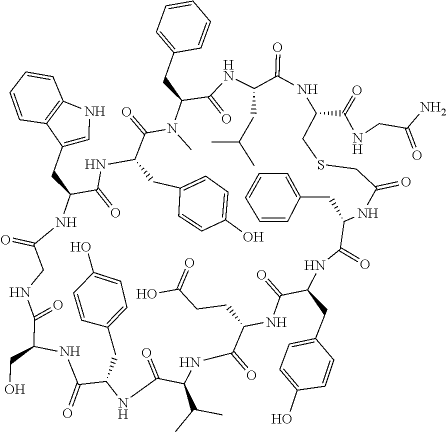 [SMILES: CC(C)C[C@@H]1NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CCC(=O)O)NC(=O)[C@H](Cc2ccc(O)cc2)NC(=O)[C@H](Cc2ccccc2)NC(=O)CSC[C@@H](C(=O)NCC(N)=O)NC1=O]

Example 1052 was prepared on a 50 μmol scale. The yield of the product was 26.5 mg, and its estimated purity by LCMS analysis was 98.1%. Analysis condition 1: Retention time=1.4 min; ESI-MS(+) m/z [M+2H]²⁺: 844.

### Preparation of Example 1053

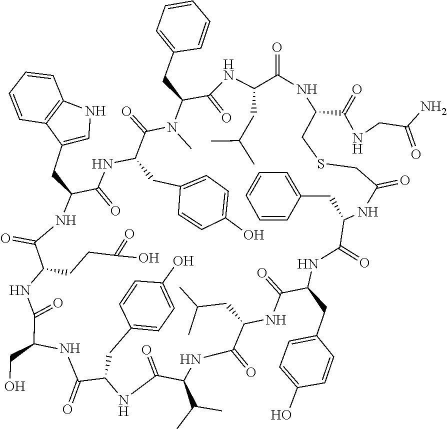 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)[C@H](CCC(=O)O)NC(=O)[C@H](CO)NC(=O)[C@H](Cc2ccc(O)cc2)NC(=O)[C@H](C(C)C)NC1=O]

Example 1053 was prepared on a 50 μmol scale. The yield of the product was 12.1 mg, and its estimated purity by LCMS analysis was 98.7%. Analysis condition 1: Retention time=1.36 min; ESI-MS(+) m/z [M+H]⁺: 1744.

### Preparation of Example 1054

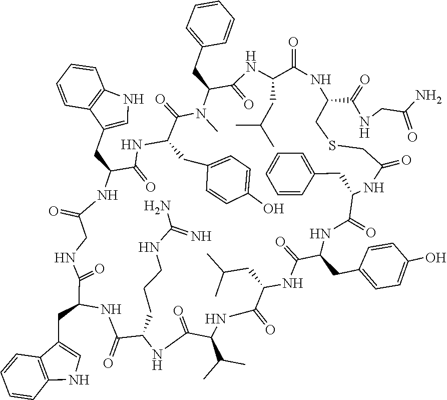 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](Cc2c[nH]c3ccccc23)NC(=O)[C@H](CCCNC(=N)N)NC(=O)[C@H](C(C)C)NC1=O]

Example 1054 was prepared on a 50 μmol scale. The yield of the product was 37.7 mg, and its estimated purity by LCMS analysis was 90.3%. Analysis condition 2: Retention time=1.55 min; ESI-MS(+) m/z [M+H]⁺: 1764.2.

### Preparation of Example 1055

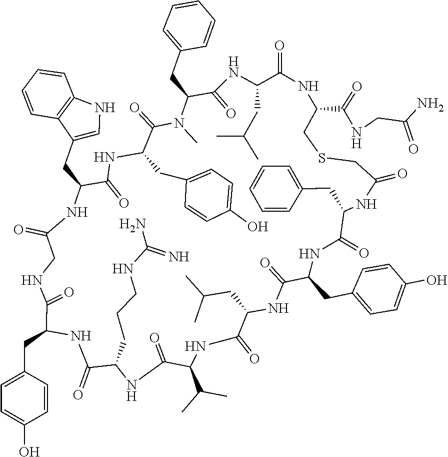 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](Cc2ccc(O)cc2)NC(=O)[C@H](CCCNC(=N)N)NC(=O)[C@H](C(C)C)NC1=O]

Example 1055 was prepared on a 50 μmol scale. The yield of the product was 24.7 mg, and its estimated purity by LCMS analysis was 96.9%. Analysis condition 1: Retention time=1.46 min; ESI-MS(+) m/z [M+H]⁺: 1740.9.

### Preparation of Example 1056

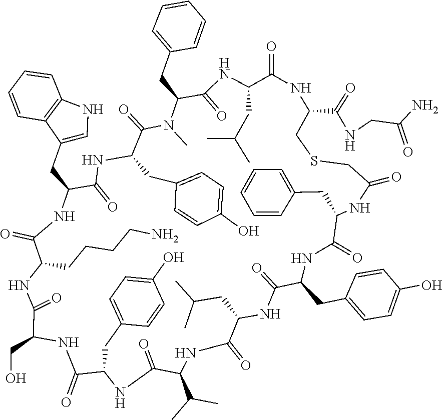 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)[C@H](CCCCN)NC(=O)[C@H](CO)NC(=O)[C@H](Cc2ccc(O)cc2)NC(=O)[C@H](C(C)C)NC1=O]

Example 1056 was prepared on a 50 μmol scale. The yield of the product was 7.4 mg, and its estimated purity by LCMS analysis was 92.9%. Analysis condition 1: Retention time=1.29 min; ESI-MS(+) m/z [M+H]⁺: 1742.

### Preparation of Example 1057

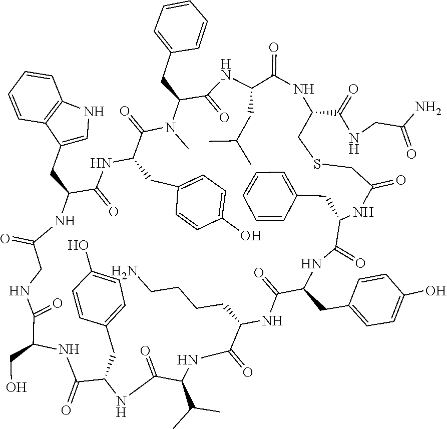 [SMILES: CC(C)C[C@@H]1NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CCCCN)NC(=O)[C@H](Cc2ccc(O)cc2)NC(=O)[C@H](Cc2ccccc2)NC(=O)CSC[C@@H](C(=O)NCC(N)=O)NC1=O]

Example 1057 was prepared on a 50 mmol scale. The yield of the product was 15.1 mg, and its estimated purity by LCMS analysis was 90.3%. Analysis condition 1: Retention time=1.38 min; ESI-MS(+) m/z [M+H]⁺: 1685.9.

### Preparation of Example 1058

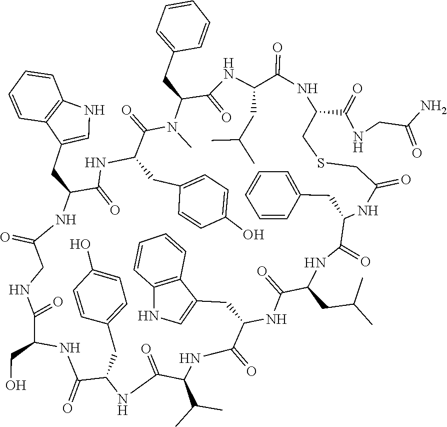 [SMILES: CC(C)C[C@@H]1NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](Cc2c[nH]c3ccccc23)NC1=O]

Example 1058 was prepared on a 50 μmol scale. The yield of the product was 9.1 mg, and its estimated purity by LCMS analysis was 97%. Analysis condition 2: Retention time=1.59 min; ESI-MS(+) m/z [M+H]⁺: 1695.2.

### Preparation of Example 1059

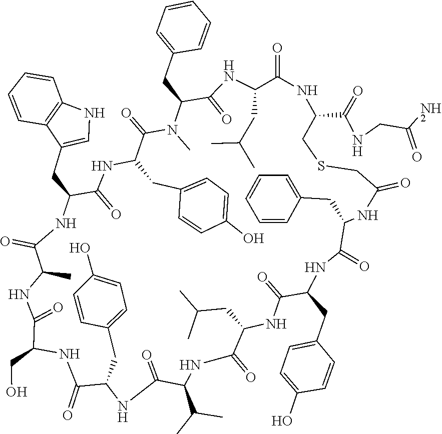

Example 1059 was prepared on a 50 μmol scale. The yield of the product was 28.4 mg, and its estimated purity by LCMS analysis was 99.3%. Analysis condition 2: Retention time=1.51 min; ESI-MS(+) m/z [M+H]⁺: 1684.9.

### Preparation of Example 1060

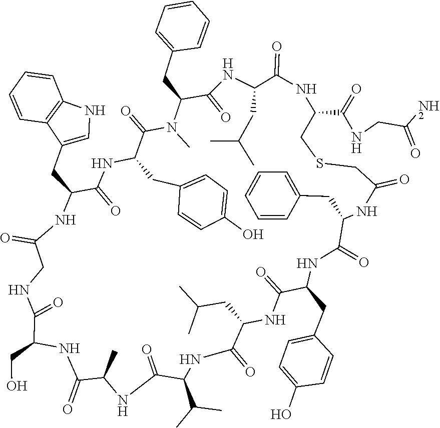 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@@H](C)NC(=O)[C@H](C(C)C)NC1=O]

Example 1060 was prepared on a 50 μmol scale. The yield of the product was 21.1 mg, and its estimated purity by LCMS analysis was 98.8%. Analysis condition 2: Retention time=1.37 min; ESI-MS(+) m/z [M+H]⁺: 1578.9.

### Preparation of Example 1061

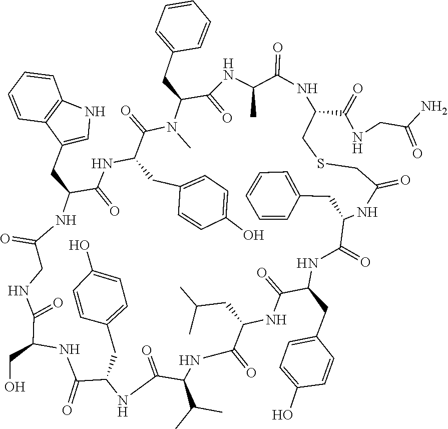 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@@H](C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1061 was prepared on a 50 μmol scale. The yield of the product was 19.1 mg, and its estimated purity by LCMS analysis was 97.6%. Analysis condition 1: Retention time=1.51 min; ESI-MS(+) m/z [M+H]⁺: 1646.1.

### Preparation of Example 1062

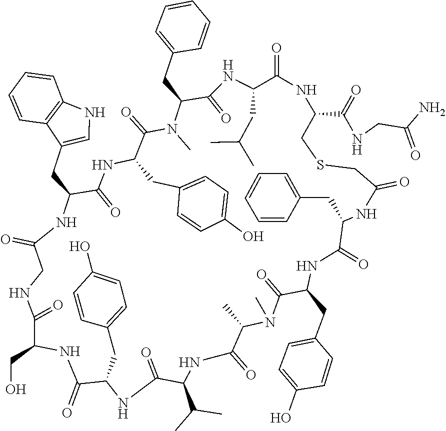 [SMILES: CC(C)C[C@@H]1NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](C)N(C)C(=O)[C@H](Cc2ccc(O)cc2)NC(=O)[C@H](Cc2ccccc2)NC(=O)CSC[C@@H](C(=O)NCC(N)=O)NC1=O]

Example 1062 was prepared on a 50 μmol scale. The yield of the product was 26.6 mg, and its estimated purity by LCMS analysis was 97.7%. Analysis condition 1: Retention time=1.49 min; ESI-MS(+) m/z [M+2H]²⁺: 821.9.

### Preparation of Example 1063

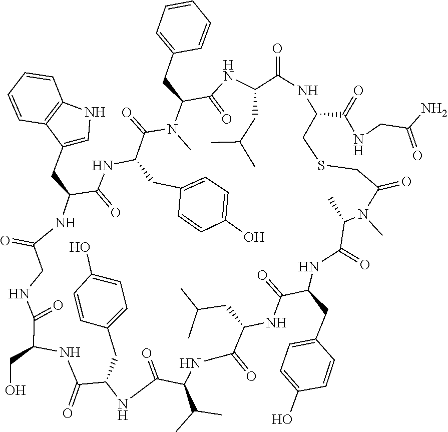 [SMILES: CC(C)C[C@@H]1NC(=O)[C@H](Cc2ccc(O)cc2)NC(=O)[C@H](C)N(C)C(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1063 was prepared on a 50 μmol scale. The yield of the product was 43.9 mg, and its estimated purity by LCMS analysis was 98.7%. Analysis condition 1: Retention time=1.46 min; ESI-MS(+) m/z [M+2H]²⁺: 805.

### Preparation of Example 1064

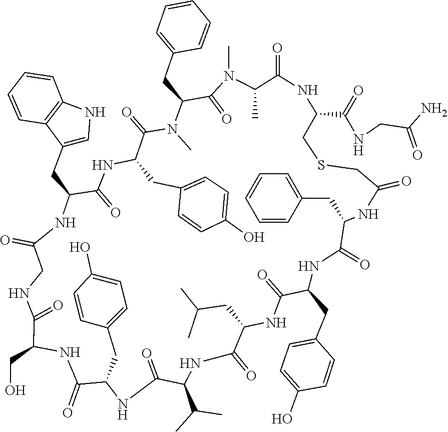 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](C)N(C)C(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1064 was prepared on a 50 μmol scale. The yield of the product was 20.9 mg, and its estimated purity by LCMS analysis was 96.9%. Analysis condition 2: Retention time=1.46 min; ESI-MS(+) m/z [M+H]⁺: 1643.

### Preparation of Example 1065

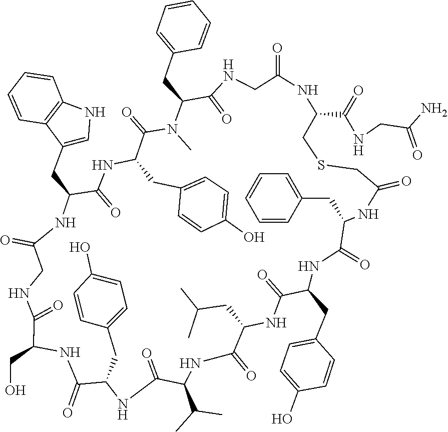 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)CNC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1065 was prepared on a 50 μmol scale. The yield of the product was 47.6 mg, and its estimated purity by LCMS analysis was 99.2%. Analysis condition 2: Retention time=1.42 min; ESI-MS(+) m/z [M+2H]²⁺: 808.

### Preparation of Example 1066

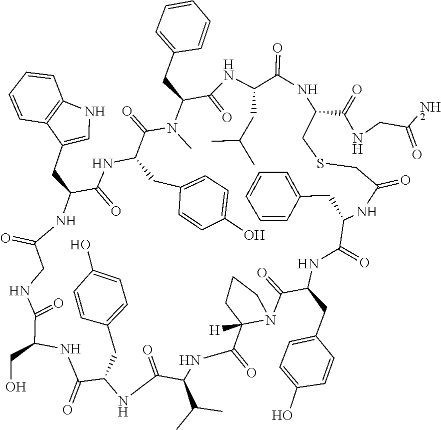 [SMILES: CC(C)C[C@@H]1NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](Cc2ccccc2)NC(=O)CSC[C@@H](C(=O)NCC(N)=O)NC1=O]

Example 1066 was prepared on a 50 mmol scale. The yield of the product was 29.3 mg, and its estimated purity by LCMS analysis was 97.3%. Analysis condition 2: Retention time=1.47 min; ESI-MS(+) m/z [M+H]⁺: 1655.

### Preparation of Example 1067

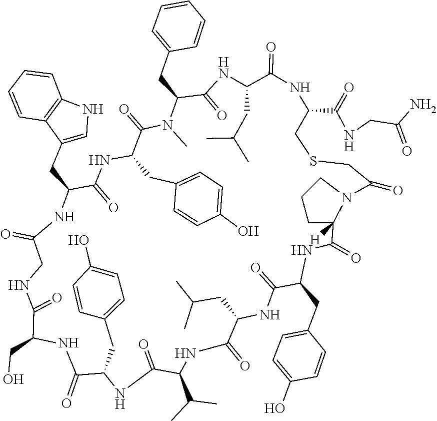 [SMILES: CC(C)C[C@@H]1NC(=O)[C@H](Cc2ccc(O)cc2)NC(=O)[C@@H]2CCCN2C(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1067 was prepared on a 50 μmol scale. The yield of the product was 16.8 mg, and its estimated purity by LCMS analysis was 98%. Analysis condition 1: Retention time=1.44 min; ESI-MS(+) m/z [M+2H]²⁺: 811.2.

### Preparation of Example 1068

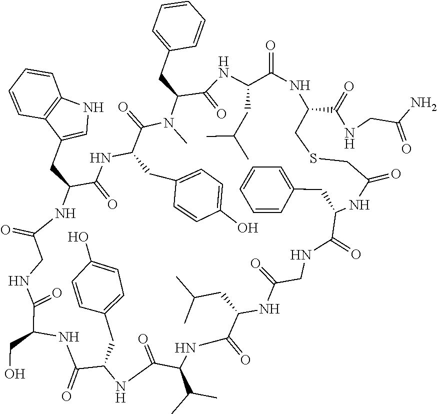 [SMILES: CC(C)C[C@@H]1NC(=O)CNC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1068 was prepared on a 50 μmol scale. The yield of the product was 24.3 mg, and its estimated purity by LCMS analysis was 97.7%. Analysis condition 2: Retention time=1.56 min; ESI-MS(+) m/z [M+2H]²⁺: 783.1.

### Preparation of Example 1069

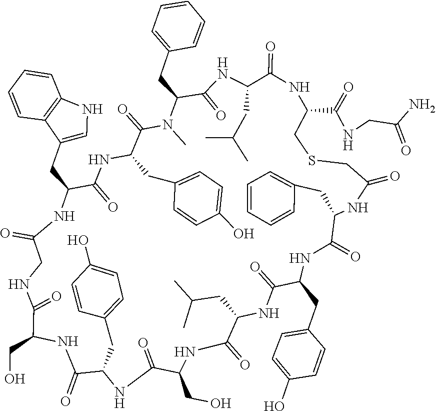 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](CO)NC1=O]

Example 1069 was prepared on a 50 μmol scale. The yield of the product was 25.9 mg, and its estimated purity by LCMS analysis was 98.7%. Analysis condition 1: Retention time=1.43 min; ESI-MS(+) m/z [M+H]⁺: 1677.1.

### Preparation of Example 1070

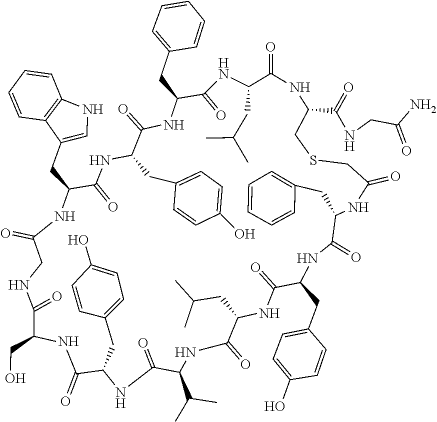 [SMILES: CC(C)C[C@@H]1NC(=O)[C@H](Cc2ccccc2)NC(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CC(C)C)NC(=O)[C@H](Cc2ccc(O)cc2)NC(=O)[C@H](Cc2ccccc2)NC(=O)CSC[C@@H](C(=O)NCC(N)=O)NC1=O]

Example 1070 was prepared on a 50 μmol scale. The yield of the product was 5.7 mg, and its estimated purity by LCMS analysis was 91.3%. Analysis condition 1: Retention time=1.51 min; ESI-MS(+) m/z [M+H]⁺: 1675.2.

### Preparation of Example 1071

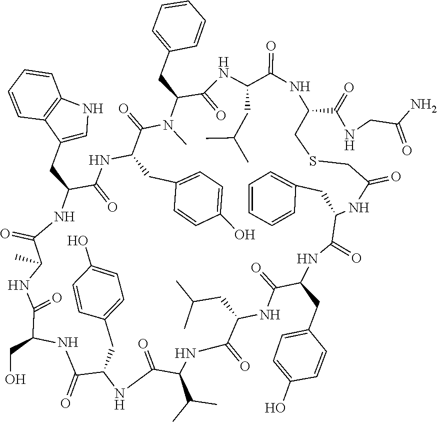 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)[C@H](C)NC(=O)[C@H](CO)NC(=O)[C@H](Cc2ccc(O)cc2)NC(=O)[C@H](C(C)C)NC1=O]

Example 1071 was prepared on a 50 μmol scale. The yield of the product was 5.3 mg, and its estimated purity by LCMS analysis was 95.7%. Analysis condition 1: Retention time=1.47 min; ESI-MS(+) m/z [M+2H]²⁺: 843.1.

### Preparation of Example 1072

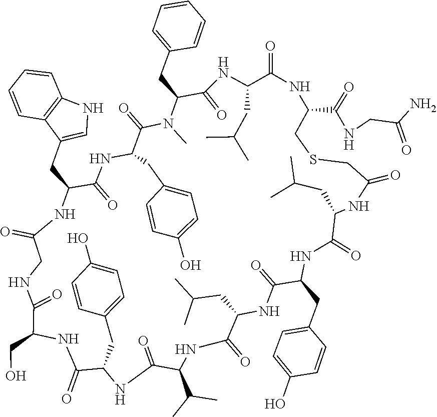 [SMILES: CC(C)C[C@@H]1NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CC(C)C)NC(=O)[C@H](Cc2ccc(O)cc2)NC1=O]

Example 1072 was prepared on a 50 μmol scale. The yield of the product was 6.9 mg, and its estimated purity by LCMS analysis was 95%. Analysis condition 2: Retention time=1.58 min; ESI-MS(+) m/z [M+H]⁺: 1636.5.

### Preparation of Example 1073

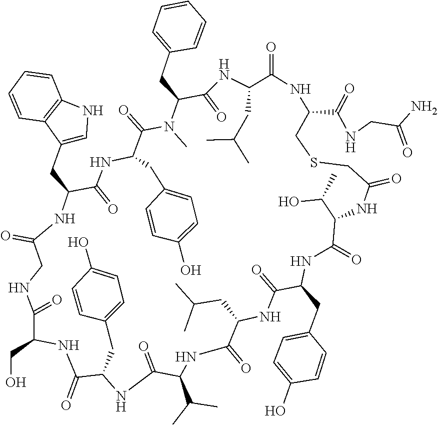 [SMILES: CC(C)C[C@@H]1NC(=O)[C@H](Cc2ccc(O)cc2)NC(=O)[C@H]([C@@H](C)O)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1073 was prepared on a 50 μmol scale. The yield of the product was 9.7 mg, and its estimated purity by LCMS analysis was 95.2%. Analysis condition 2: Retention time=1.49 min; ESI-MS(+) m/z [M+H]⁺: 1624.5.

### Preparation of Example 1074

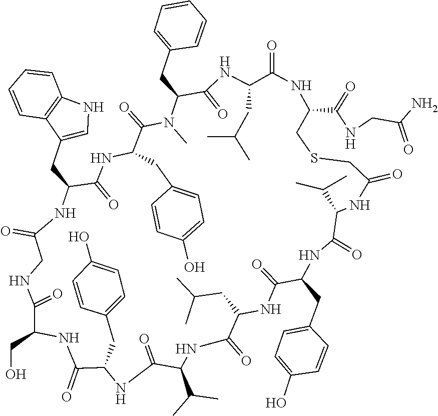

Example 1074 was prepared on a 50 μmol scale. The yield of the product was 7.8 mg, and its estimated purity by LCMS analysis was 95.5%. Analysis condition 2: Retention time=1.54 min; ESI-MS(+) m/z [M+H]⁺: 1622.5.

### Preparation of Example 1075

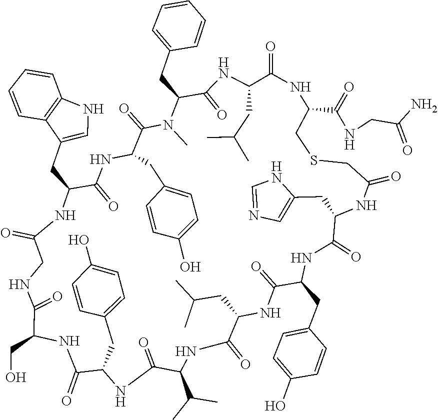 [SMILES: CC(C)C[C@@H]1NC(=O)[C@H](Cc2ccc(O)cc2)NC(=O)[C@H](Cc2cnc[nH]2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1075 was prepared on a 50 μmol scale. The yield of the product was 5.9 mg, and its estimated purity by LCMS analysis was 100%. Analysis condition 1: Retention time=1.52 min; ESI-MS(+) m/z [M+H]⁺: 1660.9.

### Preparation of Example 1076

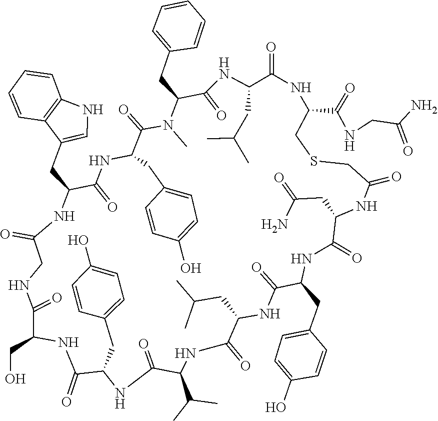 [SMILES: CC(C)C[C@@H]1NC(=O)[C@H](Cc2ccc(O)cc2)NC(=O)[C@H](CC(N)=O)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1076 was prepared on a 50 μmol scale. The yield of the product was 7.6 mg, and its estimated purity by LCMS analysis was 100%. Analysis condition 1: Retention time=1.43 min; ESI-MS(+) m/z [M+2H]²⁺: 819.2.

### Preparation of Example 1077

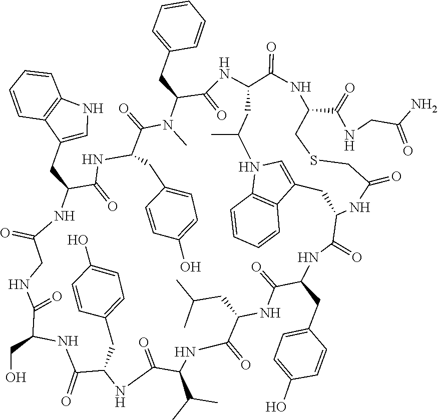 [SMILES: CC(C)C[C@@H]1NC(=O)[C@H](Cc2ccc(O)cc2)NC(=O)[C@@H]2Cc3cn(c4ccccc34)C(C)C[C@H](NC(=O)[C@H](Cc3ccccc3)N(C)C(=O)[C@H](Cc3ccc(O)cc3)NC(=O)[C@H](Cc3c[nH]c4ccccc34)NC(=O)CNC(=O)[C@H](CO)NC(=O)[C@H](Cc3ccc(O)cc3)NC(=O)[C@H](C(C)C)NC1=O)C(=O)N[C@H](C(=O)NCC(N)=O)CSCC(=O)N2]

Example 1077 was prepared on a 50 μmol scale. The yield of the product was 8.9 mg, and its estimated purity by LCMS analysis was 97.7%. Analysis condition 1: Retention time=1.34 min; ESI-MS(+) m/z [M+2H]²⁺: 856.

### Preparation of Example 1078

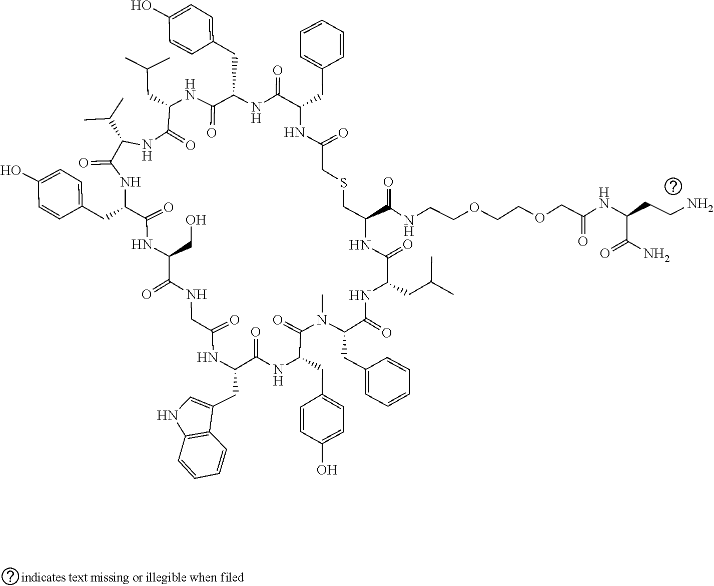 [SMILES: CC(C)C[C@@H]1NC(=O)[C@H](Cc2ccc(O)cc2)NC(=O)[C@H](Cc2ccccc2)NC(=O)CSC[C@@H](C(=O)NCCOCCOCC(=O)N[C@@H](CCN)C(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1078 was prepared on a 50 μmol scale. The yield of the product was 7.2 mg, and its estimated purity by LCMS analysis was 88.4%. Analysis condition 1: Retention time=1.46 min; ESI-MS(+) m/z [M+2H]²⁺: 944.1.

### Preparation of Example 1079

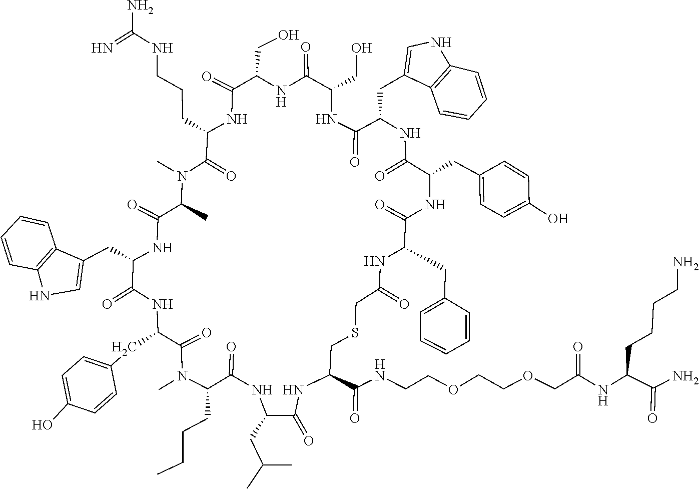 [SMILES: CCCC[C@H]1C(=O)N[C@@H](CC(C)C)C(=O)N[C@H](C(=O)NCCOCCOCC(=O)N[C@@H](CCCCN)C(N)=O)CSCC(=O)N[C@@H](Cc2ccccc2)C(=O)N[C@@H](Cc2ccc(O)cc2)C(=O)N[C@@H](Cc2c[nH]c3ccccc23)C(=O)N[C@@H](CO)C(=O)N[C@@H](CO)C(=O)N[C@@H](CCCNC(=N)N)C(=O)N(C)[C@@H](C)C(=O)N[C@@H](Cc2c[nH]c3ccccc23)C(=O)N[C@@H](Cc2ccc(O)cc2)C(=O)N1C]

Example 1079 was prepared on a 50 μmol scale. The yield of the product was 3.3 mg, and its estimated purity by LCMS analysis was 93.8%. Analysis condition 2: Retention time =1.23 min; ESI-MS(+) m/z [M+2H]²⁺: 968.1.

### Preparation of Example 1080

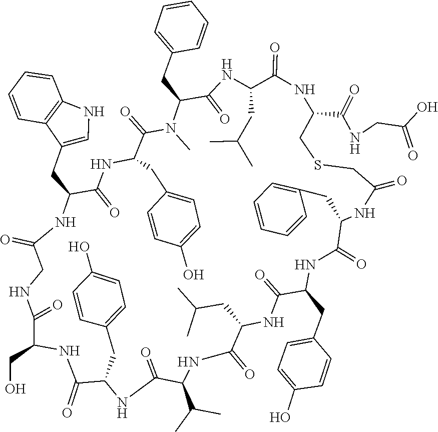 [SMILES: CC(C)C[C@@H]1NC(=O)[C@H](Cc2ccc(O)cc2)NC(=O)[C@H](Cc2ccccc2)NC(=O)CSC[C@@H](C(=O)NCC(=O)O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1080 was prepared on a 50 μmol scale. The yield of the product was 7.9 mg, and its estimated purity by LCMS analysis was 97.6%. Analysis condition 1: Retention time=1.41 min; ESI-MS(+) m/z [M+2H]²⁺: 836.4.

### Preparation of Example 1081

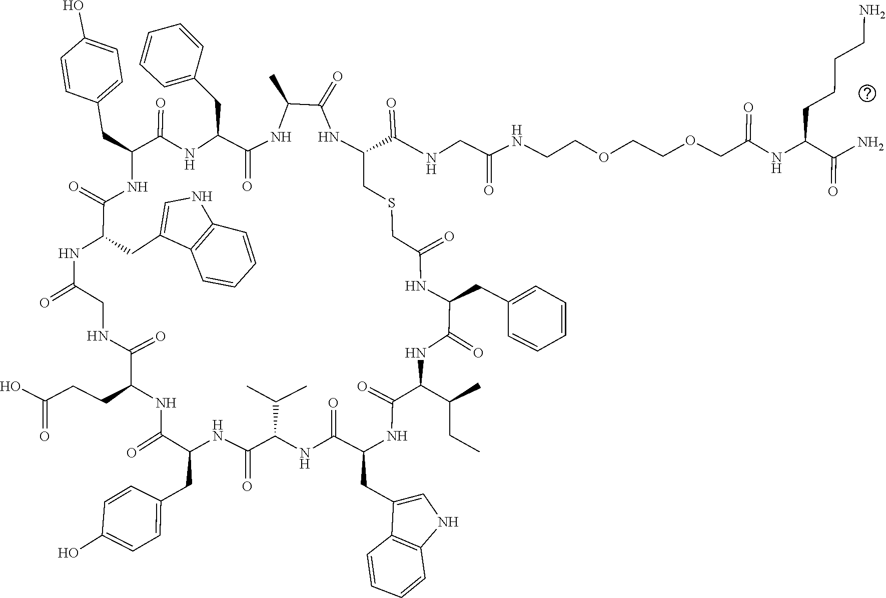 [SMILES: CC[C@H](C)[C@@H]1NC(=O)[C@H](Cc2ccccc2)NC(=O)CSC[C@@H](C(=O)NCC(=O)NCCOCCOCC(=O)N[C@@H](CCCCN)C(N)=O)NC(=O)[C@H](C)NC(=O)[C@H](Cc2ccccc2)NC(=O)[C@H](Cc2ccc(O)cc2)NC(=O)[C@H](Cc2c[nH]c3ccccc23)NC(=O)CNC(=O)[C@H](CCC(=O)O)NC(=O)[C@H](Cc2ccc(O)cc2)NC(=O)[C@H](C(C)C)NC(=O)[C@H](Cc2c[nH]c3ccccc23)NC1=O]

Example 1081 was prepared on a 50 μmol scale. The yield of the product was 1.3 mg, and its estimated purity by LCMS analysis was 93.7%. Analysis condition 1: Retention time=1.26 min; ESI-MS(+) m/z [M+2H]²⁺: 977.1.

### Preparation of Example 1082

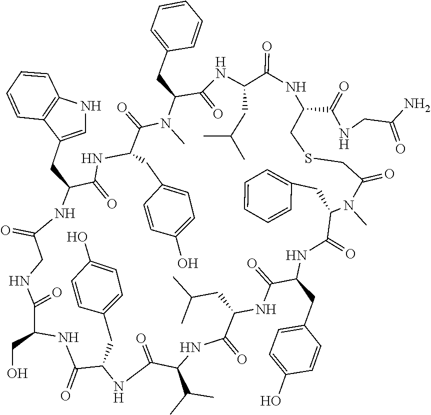 [SMILES: CC(C)C[C@@H]1NC(=O)[C@H](Cc2ccc(O)cc2)NC(=O)[C@H](Cc2ccccc2)N(C)C(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1082 was prepared on a 50 μmol scale. The yield of the product was 7.9 mg, and its estimated purity by LCMS analysis was 97%. Analysis condition 2: Retention time=1.65 min; ESI-MS(+) m/z [M+H]⁺: 1684.9.

### Preparation of Example 1083

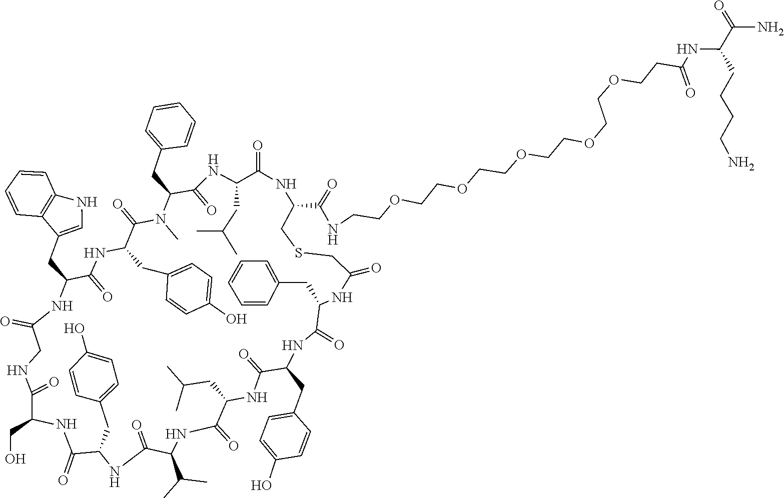 [SMILES: CC(C)C[C@@H]1NC(=O)[C@H](Cc2ccc(O)cc2)NC(=O)[C@H](Cc2ccccc2)NC(=O)CSC[C@@H](C(=O)NCCOCCOCCOCCOCCOCCC(=O)N[C@@H](CCCCN)C(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1083 was prepared on a 50 μmol scale. The yield of the product was 11.8 mg, and its estimated purity by LCMS analysis was 80.8%. Analysis condition 2: Retention time=1.41 min; ESI-MS(+) m/z [M+2H]²⁺: 1017.1.

### Preparation of Example 1084

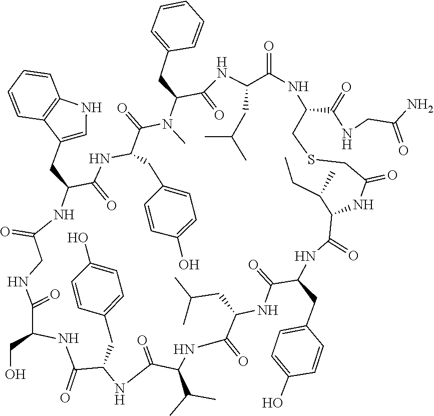 [SMILES: CC[C@H](C)[C@@H]1NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@H](CC(C)C)NC(=O)[C@H](Cc2ccc(O)cc2)NC1=O]

Example 1084 was prepared on a 50 μmol scale. The yield of the product was 7.1 mg, and its estimated purity by LCMS analysis was 89.2%. Analysis condition 2: Retention time=1.56 min; ESI-MS(+) m/z [M+H]⁺: 1636.8.

### Preparation of Example 1085

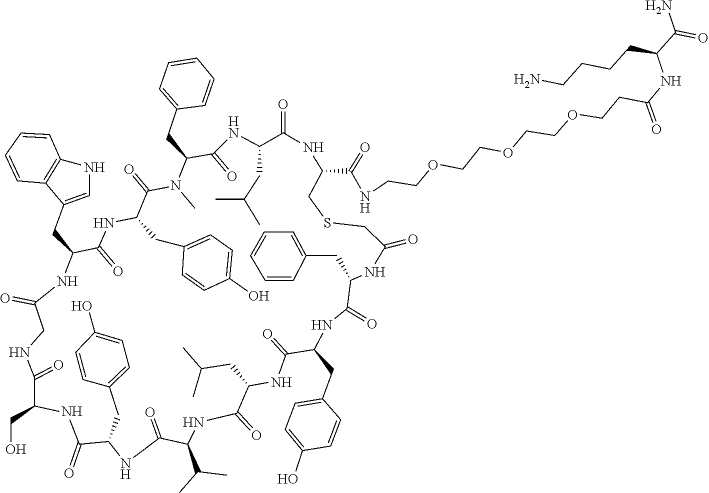 [SMILES: CC(C)C[C@@H]1NC(=O)[C@H](Cc2ccc(O)cc2)NC(=O)[C@H](Cc2ccccc2)NC(=O)CSC[C@@H](C(=O)NCCOCCOCCOCCC(=O)N[C@@H](CCCCN)C(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1085 was prepared on a 50 μmol scale. The yield of the product was 13.3 mg, and its estimated purity by LCMS analysis was 100%. Analysis condition 1: Retention time=1.47 min; ESI-MS(+) m/z [M+2H]²⁺: 973.

### Preparation of Example 1086

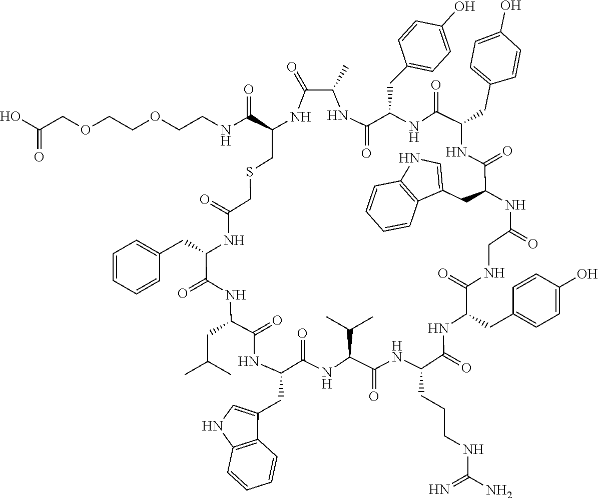 [SMILES: CC(C)C[C@@H]1NC(=O)[C@H](Cc2ccccc2)NC(=O)CSC[C@@H](C(=O)NCCOCCOCC(=O)O)NC(=O)[C@H](C)NC(=O)[C@H](Cc2ccc(O)cc2)NC(=O)[C@H](Cc2ccc(O)cc2)NC(=O)[C@H](Cc2c[nH]c3ccccc23)NC(=O)CNC(=O)[C@H](Cc2ccc(O)cc2)NC(=O)[C@H](CCCNC(=N)N)NC(=O)[C@H](C(C)C)NC(=O)[C@H](Cc2c[nH]c3ccccc23)NC1=O]

Example 1086 was prepared on a 0.1 mmol scale. The yield of the product was 1 mg, and its estimated purity by LCMS analysis was 97.3%. Analysis condition 1: Retention time=1.28 min; ESI-MS(+) m/z [M+2H]²⁺: 907.1.

### Preparation of Example 1087

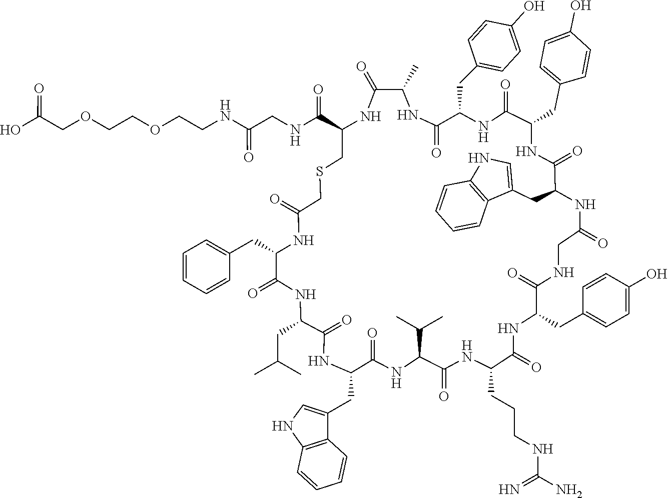 [SMILES: CC(C)C[C@@H]1NC(=O)[C@H](Cc2ccccc2)NC(=O)CSC[C@@H](C(=O)NCC(=O)NCCOCCOCC(=O)O)NC(=O)[C@H](C)NC(=O)[C@H](Cc2ccc(O)cc2)NC(=O)[C@H](Cc2ccc(O)cc2)NC(=O)[C@H](Cc2c[nH]c3ccccc23)NC(=O)CNC(=O)[C@H](Cc2ccc(O)cc2)NC(=O)[C@H](CCCNC(=N)N)NC(=O)[C@H](C(C)C)NC(=O)[C@H](Cc2c[nH]c3ccccc23)NC1=O]

Example 1087 was prepared on a 0.1 mmol scale. The yield of the product was 2 mg, and its estimated purity by LCMS analysis was 92.1%. Analysis condition 2: Retention time=1.26 min; ESI-MS(+) m/z [M+2H]²⁺: 935.2.

### Preparation of Example 1088

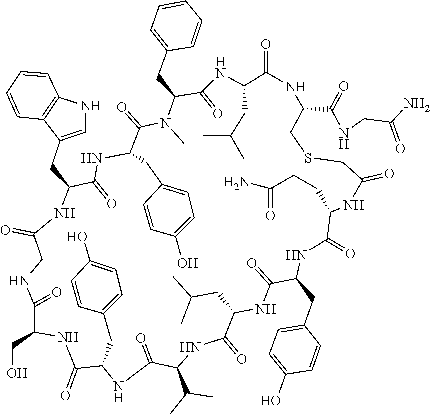 [SMILES: CC(C)C[C@@H]1NC(=O)[C@H](Cc2ccc(O)cc2)NC(=O)[C@H](CCC(N)=O)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1088 was prepared on a 50 μmol scale. The yield of the product was 12.3 mg, and its estimated purity by LCMS analysis was 99.1%. Analysis condition 2: Retention time=1.41 min; ESI-MS(+) m/z [M+2H]²⁺: 826.3.

### Preparation of Example 1089

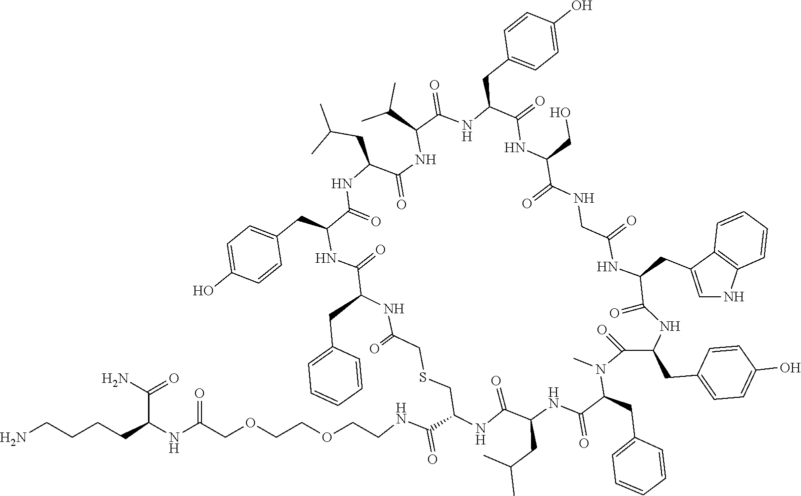 [SMILES: CC(C)C[C@@H]1NC(=O)[C@H](Cc2ccc(O)cc2)NC(=O)[C@H](Cc2ccccc2)NC(=O)CSC[C@@H](C(=O)NCCOCCOCC(=O)N[C@@H](CCCCN)C(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1089 was prepared on a 50 μmol scale. The yield of the product was 16.1 mg, and its estimated purity by LCMS analysis was 88.8%. Analysis condition 2: Retention time =1.37 min; ESI-MS(+) m/z [M+2H]²⁺: 944.2.

### Preparation of Example 1090

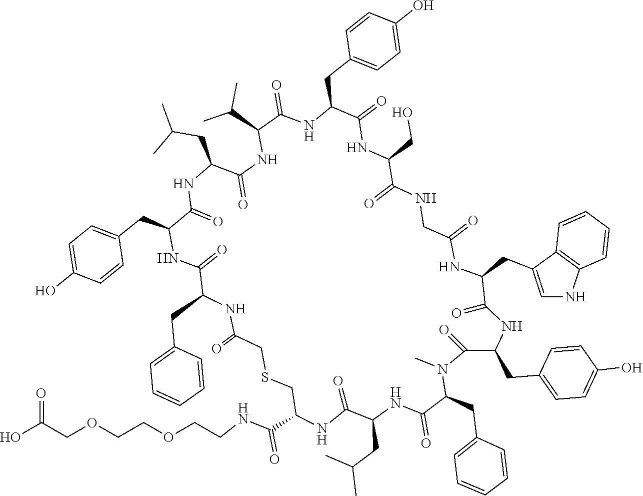 [SMILES: CC(C)C[C@@H]1NC(=O)[C@H](Cc2ccc(O)cc2)NC(=O)[C@H](Cc2ccccc2)NC(=O)CSC[C@@H](C(=O)NCCOCCOCC(=O)O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1090 was prepared on a 20 μmol scale. The yield of the product was 6.4 mg, and its estimated purity by LCMS analysis was 98.9%. Analysis condition 2: Retention time =1.6 min; ESI-MS(+) m/z [M+2H]²⁺: 881.1.

### Preparation of Example 1091

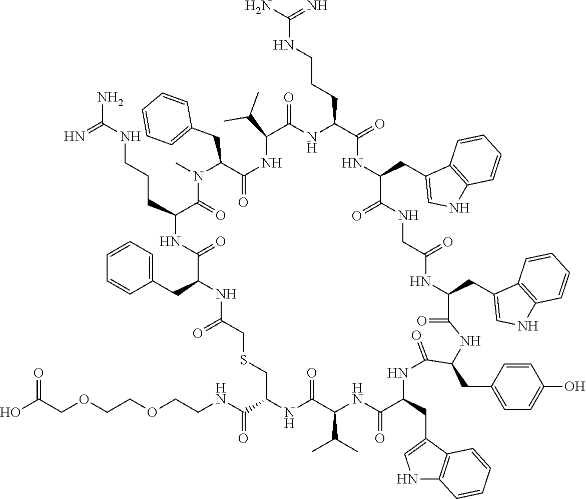 [SMILES: CC(C)[C@@H]1NC(=O)[C@H](Cc2c[nH]c3ccccc23)NC(=O)[C@H](Cc2ccc(O)cc2)NC(=O)[C@H](Cc2c[nH]c3ccccc23)NC(=O)CNC(=O)[C@H](Cc2c[nH]c3ccccc23)NC(=O)[C@H](CCCNC(=N)N)NC(=O)[C@H](C(C)C)NC(=O)[C@H](Cc2ccccc2)N(C)C(=O)[C@H](CCCNC(=N)N)NC(=O)[C@H](Cc2ccccc2)NC(=O)CSC[C@@H](C(=O)NCCOCCOCC(=O)O)NC1=O]

Example 1091 was prepared on a 0.1 mmol scale. The yield of the product was 4.1 mg, and its estimated purity by LCMS analysis was 97.6%. Analysis condition 2: Retention time=1.3 min; ESI-MS(+) m/z [M+2H]²⁺: 952.9.

### Preparation of Example 1092

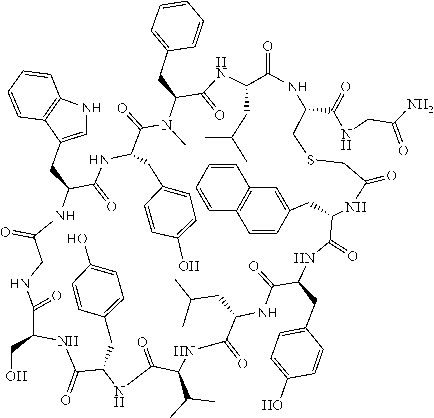 [SMILES: CC(C)C[C@@H]1NC(=O)[C@H](Cc2ccc(O)cc2)NC(=O)[C@H](Cc2ccc3ccccc3c2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1092 was prepared on a 50 μmol scale. The yield of the product was 10.6 mg, and its estimated purity by LCMS analysis was 95.3%. Analysis condition 1: Retention time=1.62 min; ESI-MS(+) m/z [M+2H]²⁺: 861.7.

### Preparation of Example 1093

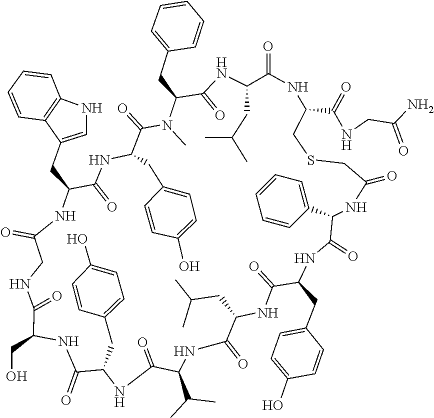 [SMILES: CC(C)C[C@@H]1NC(=O)[C@H](Cc2ccc(O)cc2)NC(=O)[C@H](c2ccccc2)NC(=O)CSC[C@@H](C(=O)NCC(N)=O)NC(=O)[C@H](CC(C)C)NC(=O)[C@H](Cc2ccccc2)N(C)C(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC1=O]

Example 1093 was prepared on a 50 μmol scale. The yield of the product was 8.9 mg, and its estimated purity by LCMS analysis was 86.4%. Analysis condition 1: Retention time=1.5 min; ESI-MS(+) m/z [M+2H]²⁺: 829.

### Preparation of Example 1094

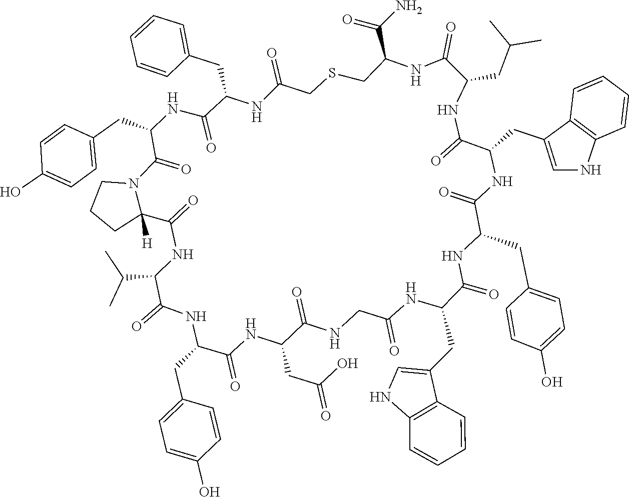 [SMILES: CC(C)C[C@@H]1NC(=O)[C@H](Cc2c[nH]c3ccccc23)NC(=O)[C@H](Cc2ccc(O)cc2)NC(=O)[C@H](Cc2c[nH]c3ccccc23)NC(=O)CNC(=O)[C@H](CC(=O)O)NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](Cc2ccccc2)NC(=O)CSC[C@@H](C(N)=O)NC1=O]

The yield of the product was 4.3 mg, and its estimated purity by LCMS analysis was 99.3%.

### Preparation of Example 1095

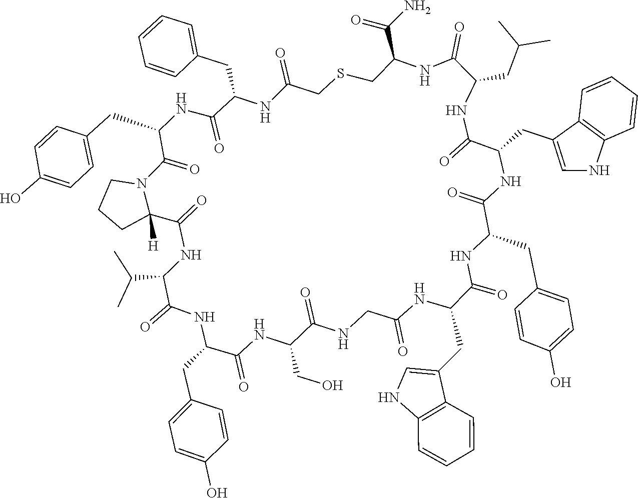 [SMILES: CC(C)C[C@@H]1NC(=O)[C@H](Cc2c[nH]c3ccccc23)NC(=O)[C@H](Cc2ccc(O)cc2)NC(=O)[C@H](Cc2c[nH]c3ccccc23)NC(=O)CNC(=O)[C@H](CO)NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](Cc2ccccc2)NC(=O)CSC[C@@H](C(N)=O)NC1=O]

The yield of the product was 4.2 mg, and its estimated purity by LCMS analysis was 94.8%.

### Preparation of Example 1096

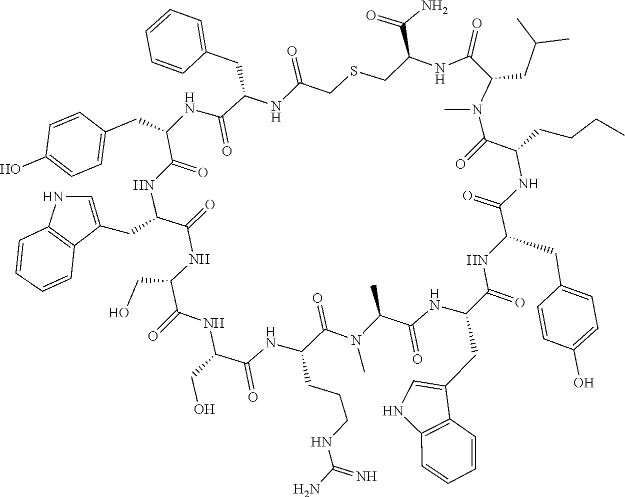 [SMILES: CCCC[C@@H]1NC(=O)[C@H](Cc2ccc(O)cc2)NC(=O)[C@H](Cc2c[nH]c3ccccc23)NC(=O)[C@H](C)N(C)C(=O)[C@H](CCCNC(=N)N)NC(=O)[C@H](CO)NC(=O)[C@H](CO)NC(=O)[C@H](Cc2c[nH]c3ccccc23)NC(=O)[C@H](Cc2ccc(O)cc2)NC(=O)[C@H](Cc2ccccc2)NC(=O)CSC[C@@H](C(N)=O)NC(=O)[C@H](CC(C)C)N(C)C1=O]

The yield of the product was 5.7 mg, and its estimated purity by LCMS analysis was 98.8%.

### Preparation of Example 1097

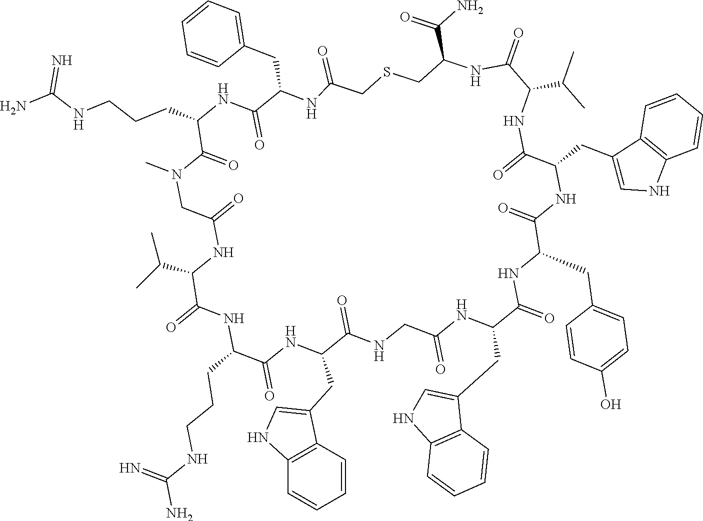 [SMILES: CC(C)[C@@H]1NC(=O)CN(C)C(=O)[C@H](CCCNC(=N)N)NC(=O)[C@H](Cc2ccccc2)NC(=O)CSC[C@@H](C(N)=O)NC(=O)[C@H](C(C)C)NC(=O)[C@H](Cc2c[nH]c3ccccc23)NC(=O)[C@H](Cc2ccc(O)cc2)NC(=O)[C@H](Cc2c[nH]c3ccccc23)NC(=O)CNC(=O)[C@H](Cc2c[nH]c3ccccc23)NC(=O)[C@H](CCCNC(=N)N)NC1=O]

The yield of the product was 4 mg, and its estimated purity by LCMS analysis was 99.1%.

### Preparation of Example 1100

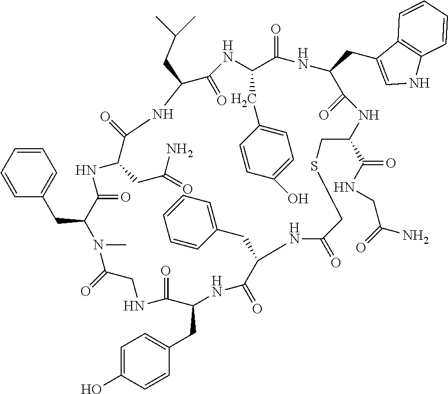 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(=O)NCC(N)=O)NC(=O)[C@H](Cc2c[nH]c3ccccc23)NC(=O)[C@H](Cc2ccc(O)cc2)NC1=O]

Example 1100 was prepared on a 100 μmol scale. The yield of the product was 54.5 mg, and its estimated purity by LCMS analysis was 97.6%. Analysis condition 1: Retention time=1.49 min; ESI-MS(+) m/z [M+H]⁺: 1322.5.

### Preparation of Example 1101

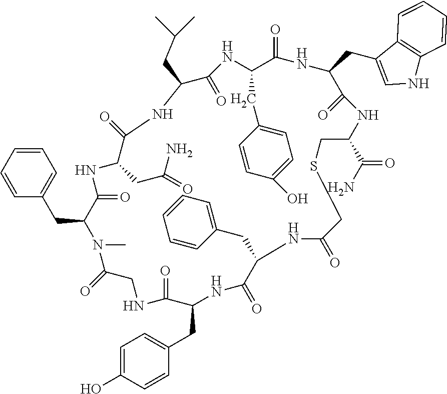 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1101 was prepared on a 100 μmol scale. The yield of the product was 53 mg, and its estimated purity by LCMS analysis was 99.2%. Analysis 25 condition 2: Retention time=1.53 min; ESI-MS(+) m/z [M+2H]²⁺: 633.2.

### Preparation of Example 1102

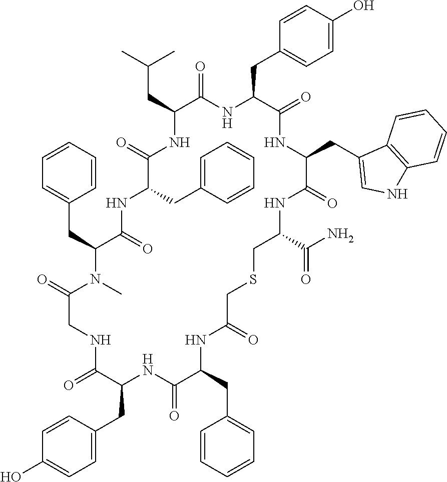 [SMILES: CC(C)C[C@@H]1NC(=O)[C@H](Cc2ccccc2)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1102 was prepared on a 100 μmol scale. The yield of the product was 39 mg, and its estimated purity by LCMS analysis was 96%. Analysis condition 2: Retention time=1.86 min; ESI-MS(+) m/z [M+H]⁺: 1298.8.

### Preparation of Example 1103

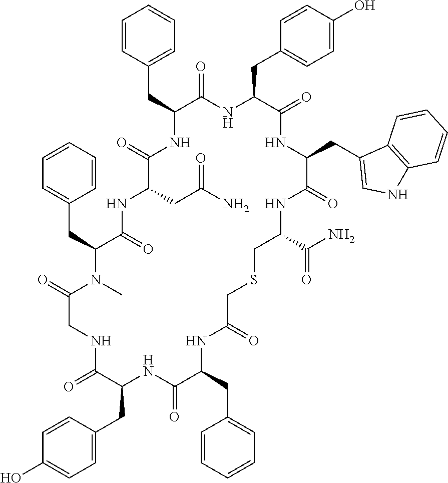 [SMILES: CN1C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC(=O)[C@H](Cc2ccccc2)NC(=O)[C@H](CC(N)=O)NC(=O)[C@@H]1Cc1ccccc1]

Example 1103 was prepared on a 100 μmol scale. The yield of the product was 50.6 mg, and its estimated purity by LCMS analysis was 89.9%. Analysis condition 1: Retention time=1.55 min; ESI-MS(+) m/z [M+H]⁺: 1299.5.

### Preparation of Example 1104

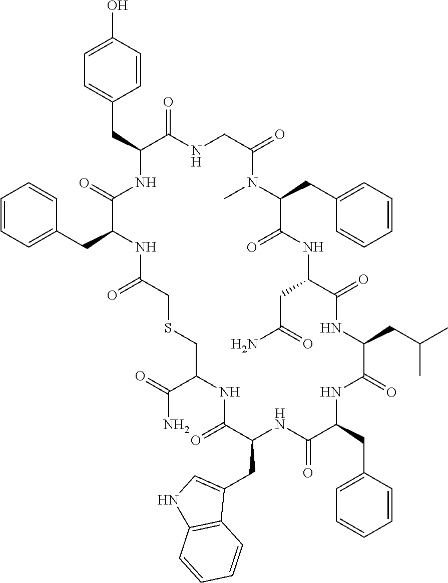 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSCC(C(N)=O)NC(=O)[C@H](Cc2c[nH]c3ccccc23)NC(=O)[C@H](Cc2ccccc2)NC1=O]

Example 1104 was prepared on a 100 μmol scale. The yield of the product was 29.7 mg, and its estimated purity by LCMS analysis was 95.5%. Analysis condition 2: Retention time=1.61 min; ESI-MS(+) m/z [M+H]⁺: 1249.7.

### Preparation of Example 1105

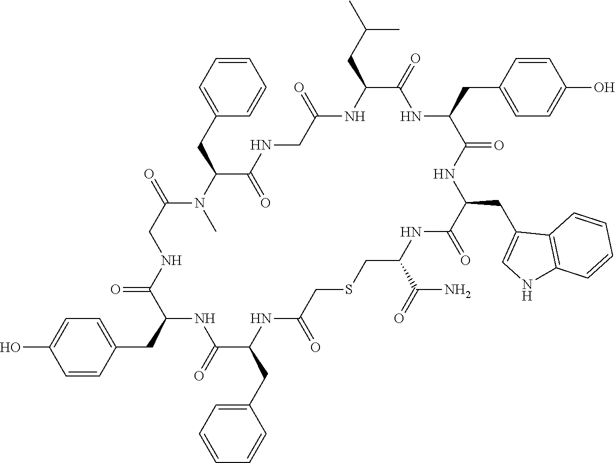 [SMILES: CC(C)C[C@@H]1NC(=O)CNC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1105 was prepared on a 50 μmol scale. The yield of the product was 9.3 mg, and its estimated purity by LCMS analysis was 96.1%. Analysis condition 2: Retention time=1.66 min; ESI-MS(+) m/z [M+H]⁺: 1208.6.

### Preparation of Example 1106

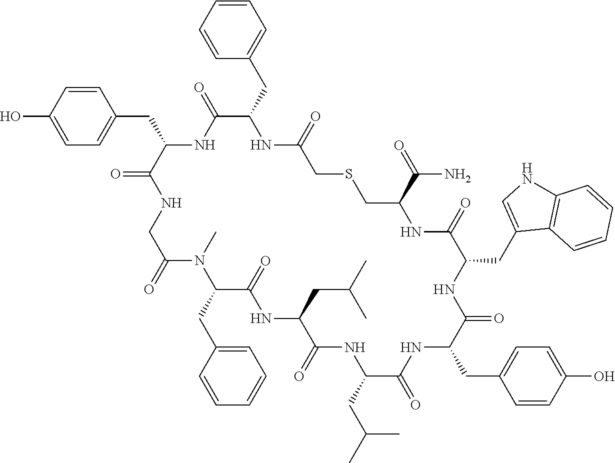 [SMILES: CC(C)C[C@@H]1NC(=O)[C@H](CC(C)C)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1106 was prepared on a 50 μmol scale. The yield of the product was 9.9 mg, and its estimated purity by LCMS analysis was 97.2%. Analysis condition 2: Retention time=1.84 min; ESI-MS(+) m/z [M+H]⁺: 1264.4.

### Preparation of Example 1107

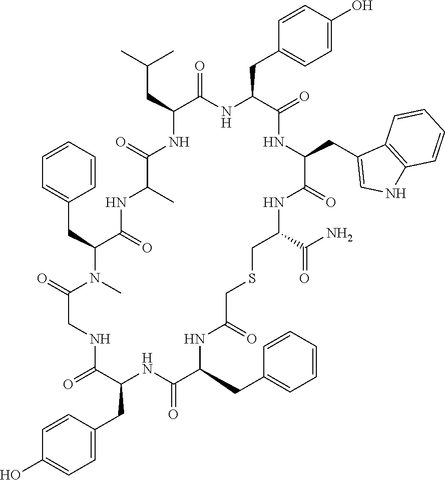 [SMILES: CC(C)C[C@@H]1NC(=O)C(C)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1107 was prepared on a 100 μmol scale. The yield of the product was 7.9 mg, and its estimated purity by LCMS analysis was 95.4%. Analysis condition 2: Retention time=1.64 min; ESI-MS(+) m/z [M+H]⁺: 1222.3.

### Preparation of Example 1108

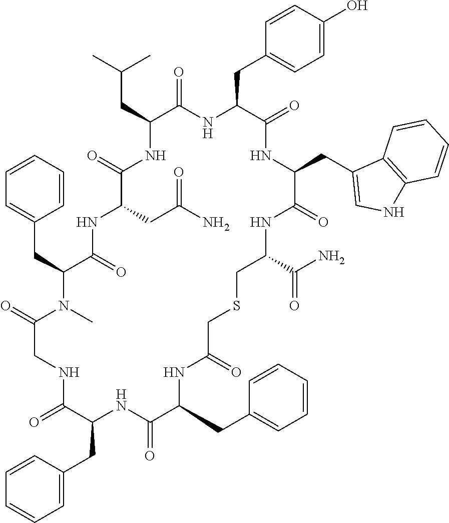 [SMILES: CC(C)C[C@@H]1NC(=O)[C@H](CC(N)=O)NC(=O)[C@H](Cc2ccccc2)N(C)C(=O)CNC(=O)[C@H](Cc2ccccc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1108 was prepared on a 100 μmol scale. The yield of the product was 5.1 mg, and its estimated purity by LCMS analysis was 100%. Analysis condition 1: Retention time=1.8 min; ESI-MS(+) m/z [M+H]⁺: 1249.3.

### Preparation of Example 1109

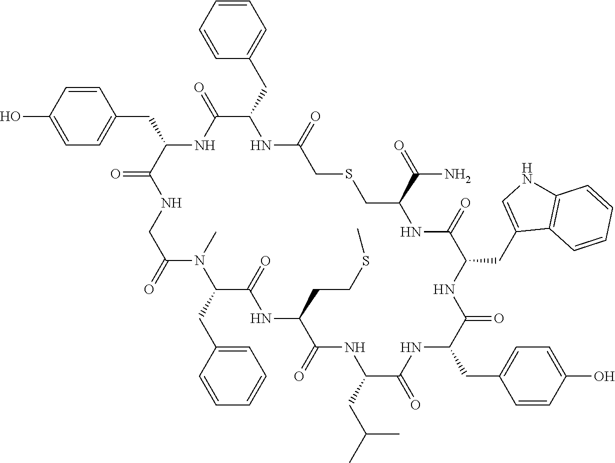 [SMILES: CSCC[C@@H]1NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC(=O)[C@H](CC(C)C)NC1=O]

The yield of the product was 25.7 mg, and its estimated purity by LCMS analysis was 100%.

### Preparation of Example 1110

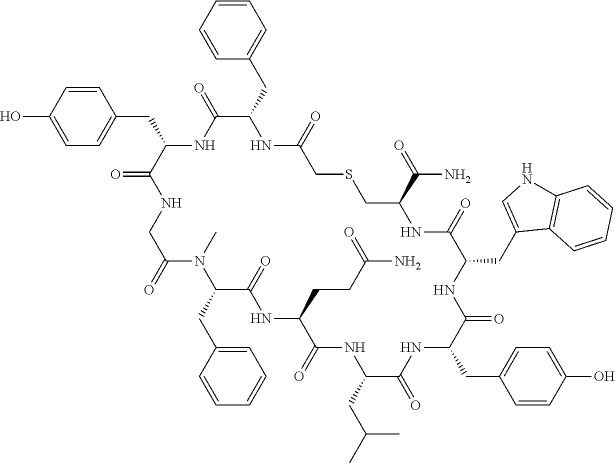 [SMILES: CC(C)C[C@@H]1NC(=O)[C@H](CCC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1110 was prepared on a 50 μmol scale. The yield of the product was 10.9 mg, and its estimated purity by LCMS analysis was 100%. Analysis condition 1: Retention time=1.66 min; ESI-MS(+) m/z [M+H]⁺: 1279.2.

### Preparation of Example 1111

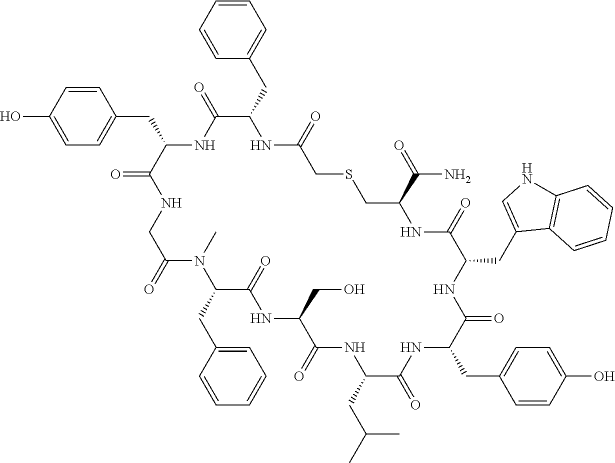 [SMILES: CC(C)C[C@@H]1NC(=O)[C@H](CO)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1111 was prepared on a 50 μmol scale. The yield of the product was 13.3 mg, and its estimated purity by LCMS analysis was 99.2%. Analysis condition 1: Retention time=1.71 min; ESI-MS(+) m/z [M+H]⁺: 1238.7.

### Preparation of Example 1112

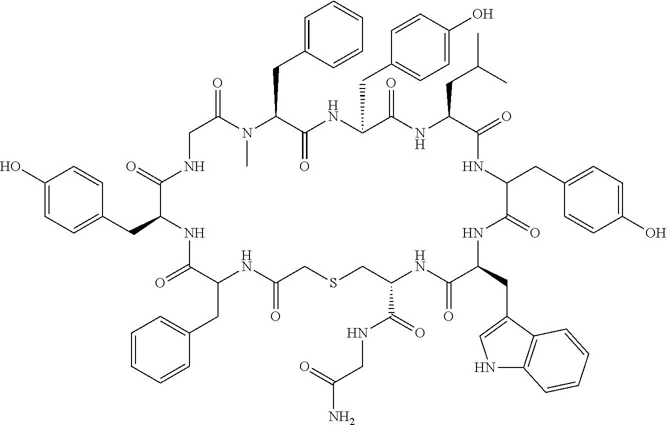 [SMILES: CC(C)C[C@@H]1NC(=O)[C@H](Cc2ccc(O)cc2)NC(=O)[C@H](Cc2ccccc2)N(C)C(=O)CNC(=O)[C@H](Cc2ccc(O)cc2)NC(=O)C(Cc2ccccc2)NC(=O)CSC[C@@H](C(=O)NCC(N)=O)NC(=O)[C@H](Cc2c[nH]c3ccccc23)NC(=O)C(Cc2ccc(O)cc2)NC1=O]

Example 1112 was prepared on a 50 μmol scale. The yield of the product was 6 mg, and its estimated purity by LCMS analysis was 97.2%. Analysis condition 1: Retention time=1.59 min; ESI-MS(+) m/z [M+H]⁺: 1371.8.

### Preparation of Example 1113

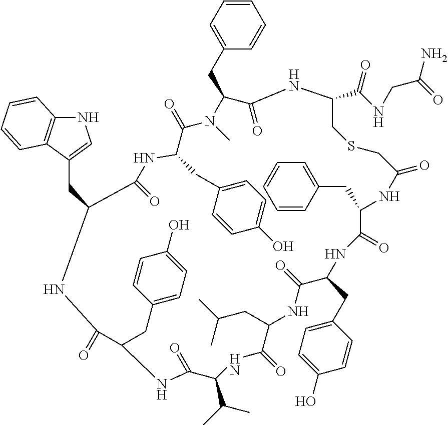 [SMILES: CC(C)CC1NC(=O)[C@H](Cc2ccc(O)cc2)NC(=O)[C@H](Cc2ccccc2)NC(=O)CSC[C@@H](C(=O)NCC(N)=O)NC(=O)[C@H](Cc2ccccc2)N(C)C(=O)[C@H](Cc2ccc(O)cc2)NC(=O)[C@H](Cc2c[nH]c3ccccc23)NC(=O)C(Cc2ccc(O)cc2)NC(=O)[C@H](C(C)C)NC1=O]

Example 1113 was prepared on a 0.1 mmol scale. The yield of the product was 21 mg, and its estimated purity by LCMS analysis was 100%. Analysis condition: Retention time=min; ESI-MS(+) m/z [M+H]⁺: 1413.7.

### Preparation of Example 1114

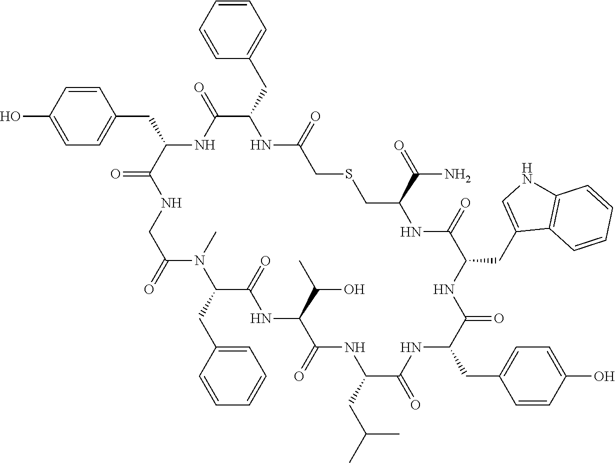 [SMILES: CC(C)C[C@@H]1NC(=O)[C@H](C(C)O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1114 was prepared on a 50 μmol scale. The yield of the product was 12.2 mg, and its estimated purity by LCMS analysis was 100%. Analysis condition 1: Retention time=1.6 min; ESI-MS(+) m/z [M+H]⁺: 1252.5.

### Preparation of Example 1115 01

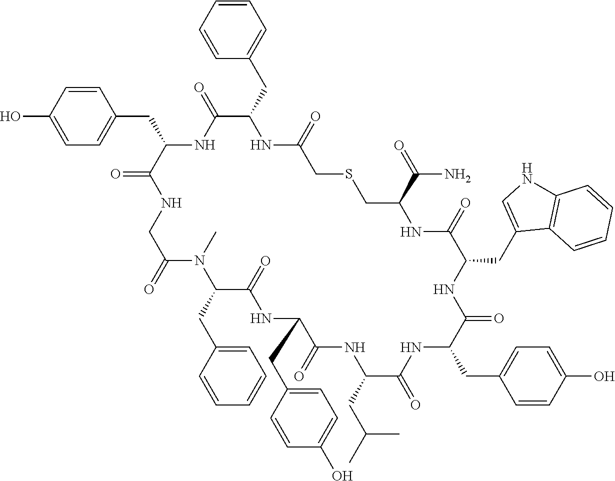 [SMILES: CC(C)C[C@@H]1NC(=O)[C@H](Cc2ccc(O)cc2)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1115 was prepared on a 50 μmol scale. The yield of the product was 24.2 mg, and its estimated purity by LCMS analysis was 100%. Analysis condition 1: Retention time=1.65 min; ESI-MS(+) m/z [M+H]⁺: 1314.2.

### Preparation of Example 1116

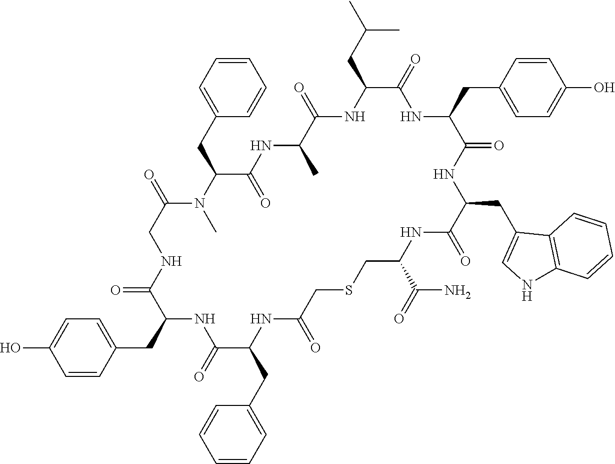 [SMILES: CC(C)C[C@@H]1NC(=O)[C@@H](C)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1116 was prepared on a 100 μmol scale. The yield of the product was 28.5 mg, and its estimated purity by LCMS analysis was 96.3%. Analysis condition 1: Retention time=1.67 min; ESI-MS(+) m/z [M+H]⁺: 1222.5.

### Preparation of Example 1117

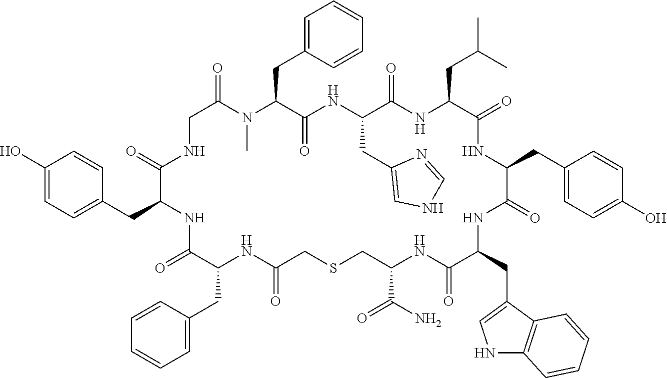 [SMILES: CC(C)C[C@@H]1NC(=O)[C@H](Cc2c[nH]cn2)NC(=O)[C@H](Cc2ccccc2)N(C)C(=O)CNC(=O)[C@H](Cc2ccc(O)cc2)NC(=O)[C@@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1117 was prepared on a 100 μmol scale. The yield of the product was 17.4 mg, and its estimated purity by LCMS analysis was 91.9%. Analysis condition 1: Retention time=1.51 min; ESI-MS(+) m/z [M+H]⁺: 1288.7.

### Preparation of Example 1118

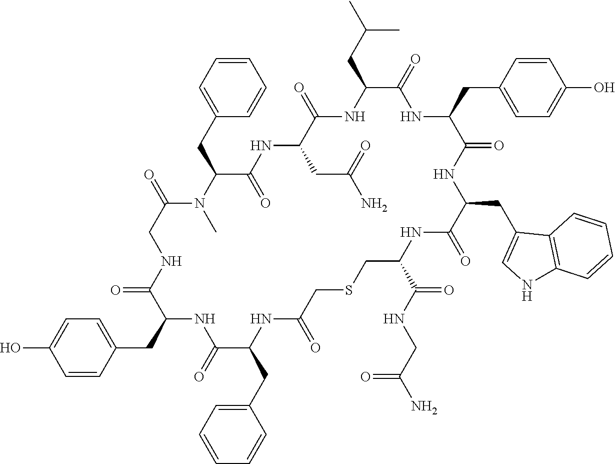 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(=O)NCC(N)=O)NC(=O)[C@H](Cc2c[nH]c3ccccc23)NC(=O)[C@H](Cc2ccc(O)cc2)NC1=O]

Example 1118 was prepared on a 100 μmol scale. The yield of the product was 15.2 mg, and its estimated purity by LCMS analysis was 99.5%. Analysis condition 2: Retention time=1.49 min; ESI-MS(+) m/z [M+2H]²⁺: 661.4.

### Preparation of Example 1119

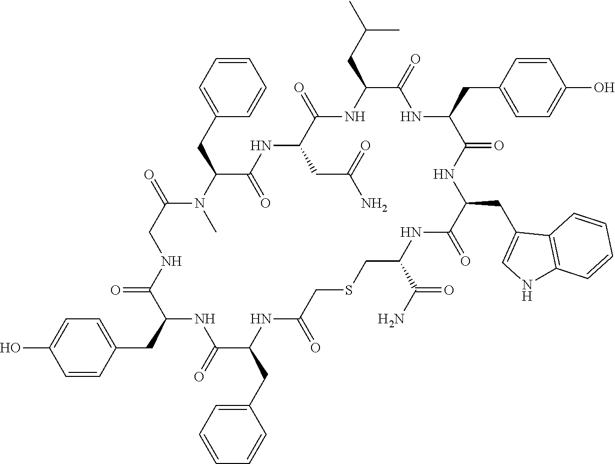 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1119 was prepared on a 100 μmol scale. The yield of the product was 10.4 mg, and its estimated purity by LCMS analysis was 100%. Analysis condition 1: Retention time=1.52 min; ESI-MS(+) m/z [M+H]⁺: 1265.4.

### Preparation of Example 1120

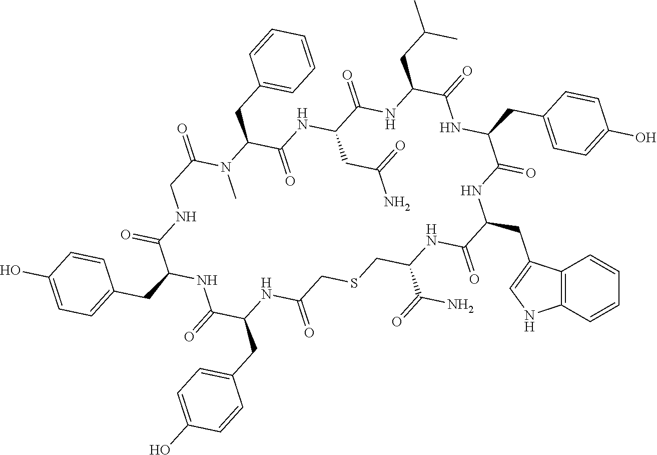 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccc(O)cc2)NC(=O)CSC[C@@H](C(N)=O)NC(=O)[C@H](Cc2c[nH]c3ccccc23)NC(=O)[C@H](Cc2ccc(O)cc2)NC1=O]

Example 1120 was prepared on a 50 μmol scale. The yield of the product was 6.6 mg, and its estimated purity by LCMS analysis was 95.6%. Analysis condition 2: Retention time=1.39, 1.41 min; ESI-MS(+) m/z [M+H]⁺: 1281.78.

### Preparation of Example 1121

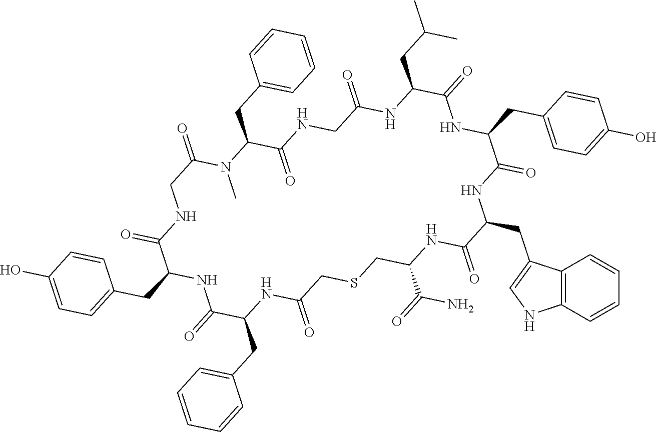 [SMILES: CC(C)C[C@@H]1NC(=O)CNC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1121 was prepared on a 50 μmol scale. The yield of the product was 15.8 mg, and its estimated purity by LCMS analysis was 97.2%. Analysis condition 2: Retention time=1.63 min; ESI-MS(+) m/z [M+H]⁺: 1208.5.

### Preparation of Example 1122

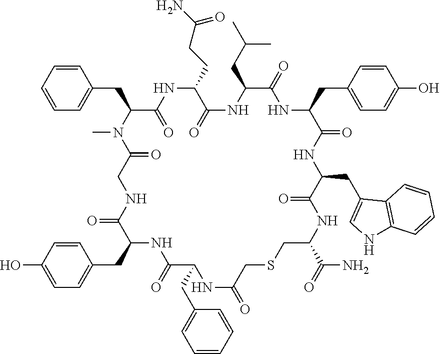 [SMILES: CC(C)C[C@@H]1NC(=O)[C@@H](CCC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1122 was prepared on a 50 μmol scale. The yield of the product was 3.1 mg, and its estimated purity by LCMS analysis was 89.1%. Analysis condition 1: Retention time=1.53 min; ESI-MS(+) m/z [M+H]⁺: 1279.7.

### Preparation of Example 1123

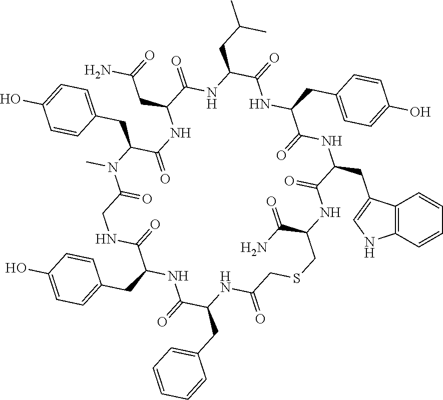 [SMILES: CC(C)C[C@@H]1NC(=O)[C@H](CC(N)=O)NC(=O)[C@H](Cc2ccc(O)cc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

Example 1123 was prepared on a 50 μmol scale. The yield of the product was 25.9 mg, and its estimated purity by LCMS analysis was 100%. Analysis condition 1: Retention time=1.34 min; ESI-MS(+) m/z [M+H]⁺: 1281.9.

### Preparation of Example 1124

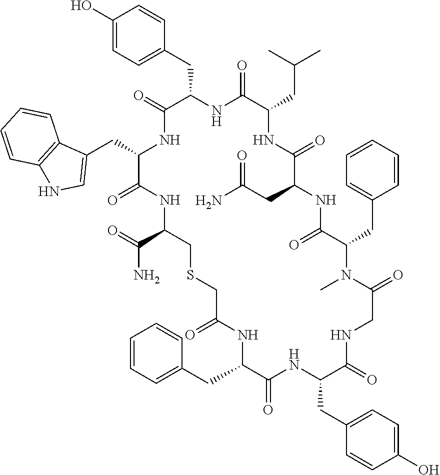 [SMILES: CC(C)C[C@@H]1NC(=O)[C@H](CC(N)=O)NC(=O)[C@H](Cc2ccccc2)N(C)C(=O)CNC(=O)[C@H](Cc2ccc(O)cc2)NC(=O)[C@H](Cc2ccccc2)NC(=O)CSC[C@@H](C(N)=O)NC(=O)[C@H](Cc2c[nH]c3ccccc23)NC(=O)[C@H](Cc2ccc(O)cc2)NC1=O]

The yield of the product was 10.9 mg, and its estimated purity by LCMS analysis was 99.4%.

### Preparation of Example 1200

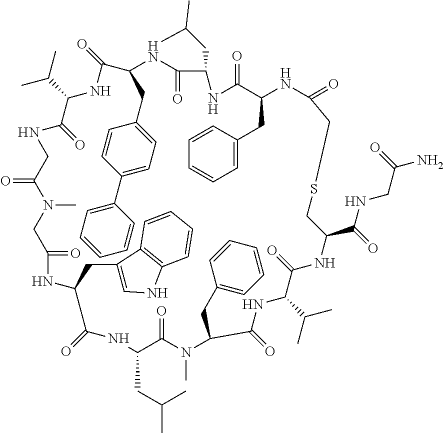

Example 1200 was prepared on a 100 μmol scale. The yield of the product was 45.2 mg, and its estimated purity by LCMS analysis was 97.4%. Analysis condition 1: Retention time=1.83 min; ESI-MS(+) m/z [M+H]⁺: 1487.9.

### Preparation of Example 1201

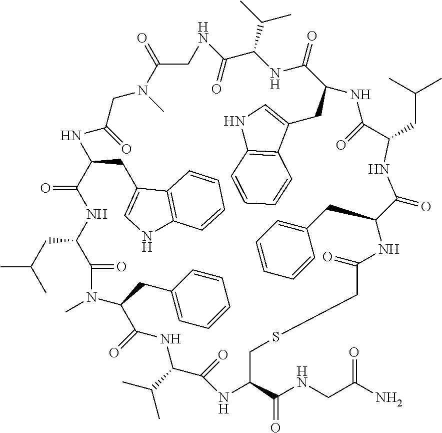 [SMILES: CC(C)C[C@@H]1NC(=O)[C@H](Cc2ccccc2)NC(=O)CSC[C@@H](C(=O)NCC(N)=O)NC(=O)[C@H](C(C)C)NC(=O)[C@H](Cc2ccccc2)N(C)C(=O)[C@H](CC(C)C)NC(=O)[C@H](Cc2c[nH]c3ccccc23)NC(=O)CN(C)C(=O)CNC(=O)[C@H](C(C)C)NC(=O)[C@H](Cc2c[nH]c3ccccc23)NC1=O]

Example 1201 was prepared on a 100 μmol scale. The yield of the product was 14.9 mg, and its estimated purity by LCMS analysis was 96.6%. Analysis condition 2: Retention time=1.58 min; ESI-MS(+) m/z [M+2H]²⁺: 726.

### Preparation of Example 1202

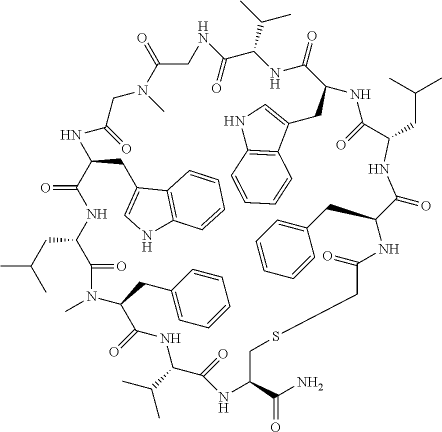 [SMILES: CC(C)C[C@@H]1NC(=O)[C@H](Cc2ccccc2)NC(=O)CSC[C@@H](C(N)=O)NC(=O)[C@H](C(C)C)NC(=O)[C@H](Cc2ccccc2)N(C)C(=O)[C@H](CC(C)C)NC(=O)[C@H](Cc2c[nH]c3ccccc23)NC(=O)CN(C)C(=O)CNC(=O)[C@H](C(C)C)NC(=O)[C@H](Cc2c[nH]c3ccccc23)NC1=O]

Example 1202 was prepared on a 100 μmol scale. The yield of the product was 28.8 mg, and its estimated purity by

LCMS analysis was 92.2%. Analysis condition 2: Retention time=1.69 min; ESI-MS(+) m/z [M+H]⁺: 1394.

### Preparation of Example 1203

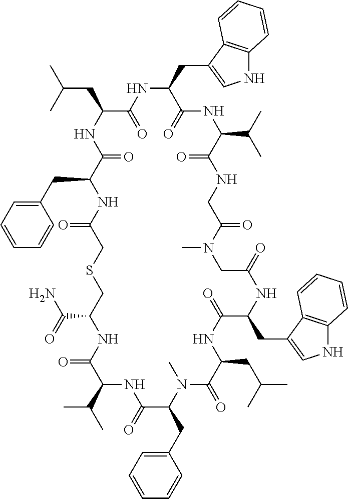 [SMILES: CC(C)C[C@@H]1NC(=O)[C@H](Cc2ccccc2)NC(=O)CSC[C@@H](C(N)=O)NC(=O)[C@H](C(C)C)NC(=O)[C@H](Cc2ccccc2)N(C)C(=O)[C@H](CC(C)C)NC(=O)[C@H](Cc2c[nH]c3ccccc23)NC(=O)CN(C)C(=O)CNC(=O)[C@H](C(C)C)NC(=O)[C@H](Cc2c[nH]c3ccccc23)NC1=O]

The yield of the product was 9.8 mg, and its estimated purity by LCMS analysis was 99%.

### Preparation of Example 1204

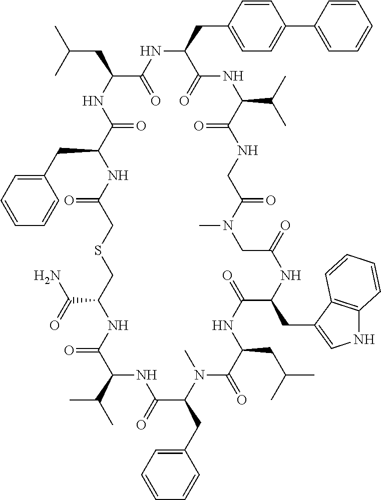 [SMILES: CC(C)C[C@@H]1NC(=O)[C@H](Cc2ccccc2)NC(=O)CSC[C@@H](C(N)=O)NC(=O)[C@H](C(C)C)NC(=O)[C@H](Cc2ccccc2)N(C)C(=O)[C@H](CC(C)C)NC(=O)[C@H](Cc2c[nH]c3ccccc23)NC(=O)CN(C)C(=O)CNC(=O)[C@H](C(C)C)NC(=O)[C@H](Cc2ccc(-c3ccccc3)cc2)NC1=O]

The yield of the product was 5.7 mg, and its estimated purity by LCMS analysis was 98.5%.

### Preparation of Example 1205

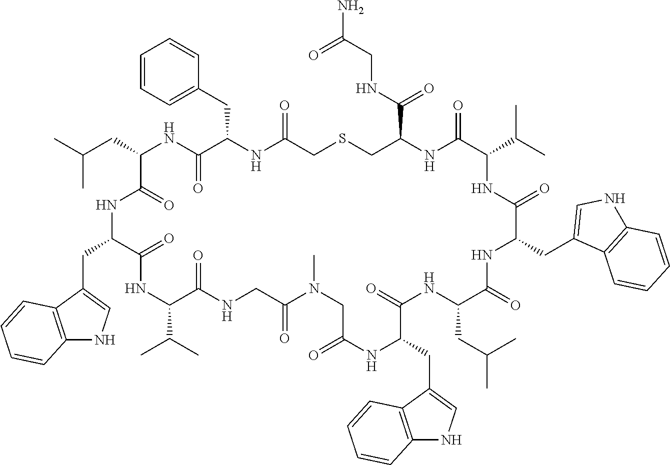 [SMILES: CC(C)C[C@@H]1NC(=O)[C@H](Cc2ccccc2)NC(=O)CSC[C@@H](C(=O)NCC(N)=O)NC(=O)[C@H](C(C)C)NC(=O)[C@H](Cc2c[nH]c3ccccc23)NC(=O)[C@H](CC(C)C)NC(=O)[C@H](Cc2c[nH]c3ccccc23)NC(=O)CN(C)C(=O)CNC(=O)[C@H](C(C)C)NC(=O)[C@H](Cc2c[nH]c3ccccc23)NC1=O]

Example 1205 was prepared on a 50 μmol scale. The yield of the product was 2.7 mg, and its estimated purity by

LCMS analysis was 99%. Analysis condition 1: Retention time=1.59 min; ESI-MS(+) m/z [M+H]⁺: 1475.8.

### Preparation of Example 1206

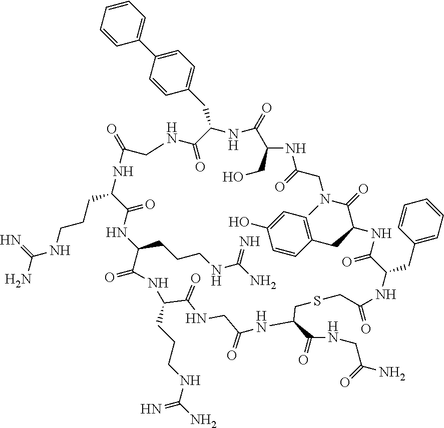 [SMILES: CN1CC(=O)N[C@@H](CO)C(=O)N[C@@H](Cc2ccc(-c3ccccc3)cc2)C(=O)NCC(=O)N[C@@H](CCCNC(=N)N)C(=O)N[C@@H](CCCNC(=N)N)C(=O)N[C@@H](CCCNC(=N)N)C(=O)NCC(=O)N[C@H](C(=O)NCC(N)=O)CSCC(=O)N[C@@H](Cc2ccccc2)C(=O)N[C@@H](Cc2ccc(O)cc2)C1=O]

Example 1206 was prepared on a 100 μmol scale. The yield of the product was 23.4 mg, and its estimated purity by LCMS analysis was 96.2%. Analysis condition 1: Retention time=1.21 min; ESI-MS(+) m/z [M+2H]²⁺: 747.

### Example 1300

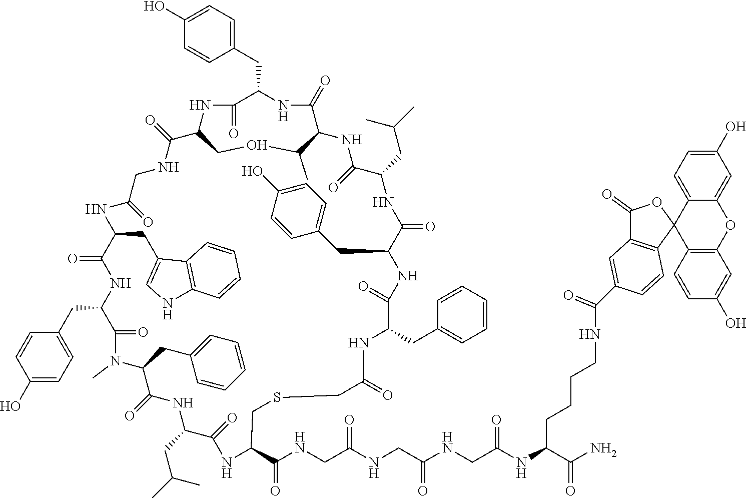

Capping with 5-carboxyfluorescein: To a solution of (3R,6S,9S,12S,15S,21S,24S,27S,30S,33S,36S)-15-((1H-indol-3-yl)methyl)-9,36-dibenzyl-N-(2-((2-((2-(((S)-1,6-diamino- l-oxohexan-2-yl)amino)-2-oxoethyl)amino)-2-oxoethyl)amino)-2-oxoethyl)-12,24,33-tris(4-hydroxybenzyl)-21-(hydroxymethyl)-6,30-diisobutyl-27-isopropyl-10-methyl-5,8,11,14,17,20,23,26,29,32,35,38-dodecaoxo-1-thia-4,7,10,13,16,19,22,25,28,31,34,37-dodecaazacyclononatriacontane-3-carboxamide (38.3 mg, 0.020 mmol) in DMF (2 mL) was added a solution of 5-carboxyfluorescein (15.05 mg, 0.040 mmol), and 1-hydroxy-7-azabenzotriazole (5.44 mg, 0.040 mmol) in DMF (1 mL) followed by the addition of N,N′-diisopropylcarbodiimide (6.28 μL, 0.040 mmol). The reaction was shaken at rt for 24 h.

The reaction was acidified with acetic acid. The red-yellow reaction was filtered through a 0.45 micron filter, and purified on a prep HPLC: Gradient Time: 50 min, Flow Rate: 15 ml/min, FRC Collection Time: 0.35 to 50 min; Start % B: 15, Final % B: 70; Solvent A: 0.1% TFA in Water, Solvent B: 0.1% TFA in acetonitrile; Column: Phen Luna 5u C18(2) 100A 250×21.2 mm AXIA packed (10-100 mg) #:520221-2; Gradient Collection By: UV; Wavelength: 217, Injection Vol: 1000 uL. Fractions were checked by HPLC and LCMS. Clean fractions were combined and lyophilized to give the desired capped product as a yellow lyophilite. LCMS:[M+2H]²⁺=1136.5. [M+Na]⁺=2293.7.

### Example 1400

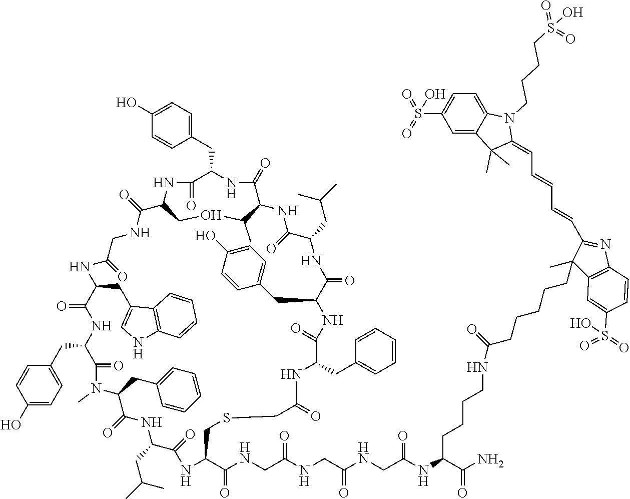

To a solution of (3R,6S,9S,12S,15S,21S,24S,27S,30S,33S,36S)-15-((1H-indol-3-yl)methyl)-9,36-dibenzyl-N-(2-((2-((2- (((S)-1,6-diamino-1-oxohexan-2-yl)amino)-2-oxoethyl)amino)-2-oxoethyl)amino)-2-oxoethyl)-12,24,33-tris(4-hydroxybenzyl)-21-(hydroxymethyl)-6,30-diisobutyl-27- isopropyl-10-methyl-5,8,11,14,17,20,23,26,29,32,35,38-dodecaoxo-1-thia-4,7,10,13,16,19,22,25,28,31,34,37-dodecaazacyclononatriacontane-3-carboxamide, TFA (2.352 mg, 1.16 μmol) in DMF (300 μL) was added N,N-diisopropylethylamine (0.596 μl, 3.48 μmol) in a dark hood. The dye, 2-((1E,3E,5E)-5-(3,3-dimethyl-5-sulfo-1-(4-sulfobutyl)indolin-2-ylidene)penta-1,3-dien-1-yl)-3-(6-((2,5-dioxopyrrolidin-1- yl)oxy)-6-oxohexyl)-3-methyl-3H-indole-5-sulfonic acid (0.984 mg, 1.16 μmol) was dissolved in DMSO (100 μL) in the dark and was added to the reaction. The vial was wrapped in aluminum foil and shaken in the dark. After 1 hour, an additional peptide (0.5 mg) was added, and let shaken overnight. LCMS indicated the desired product only. The reaction was concentrated in vacuo to remove the organic solvents. The blue residue was triturated with ether (2×2 mL×5 min), no blue color observed. The residue was dissolved in water (1 mL), and transferred into a tarred vial, rinsed the vial with water (2×1 mL), and then transferred to a the tarred vial, and lyophilized to give the desired product 3-((S)-1-((3R,6S,9S,12S,15S,21S,24S,27S,30S,33S,36S)-15-((1H-indol-3-yl)methyl)-9,36-dibenzyl-12,24,33-tris(4- hydroxybenzyl)-21-(hydroxymethyl)-6,30-diisobutyl-27-isopropyl-10-methyl-5,8,11,14,17,20,23,26,29,32,35,38-dodecaoxo-1-thia-4,7,10,13,16,19,22,25,28,31,34,37-dodecaazacyclononatriacontan- 3-yl)-12-carbamoyl-1,4,7,10,18-pentaoxo-2,5,8,11,17-pentaazatricosan-23-yl)-2-((lE,3E,5E)-5-(3,3-dimethyl-5-sulfo-1-(4-sulfobutyl)indolin-2- ylidene)penta-1,3-dien-1-yl)-3-methyl-3H-indole-5-sulfonic acid (2.7 mg, 83% yield). LCMS:[M+2H]2+=1323.7.

### Example 1500

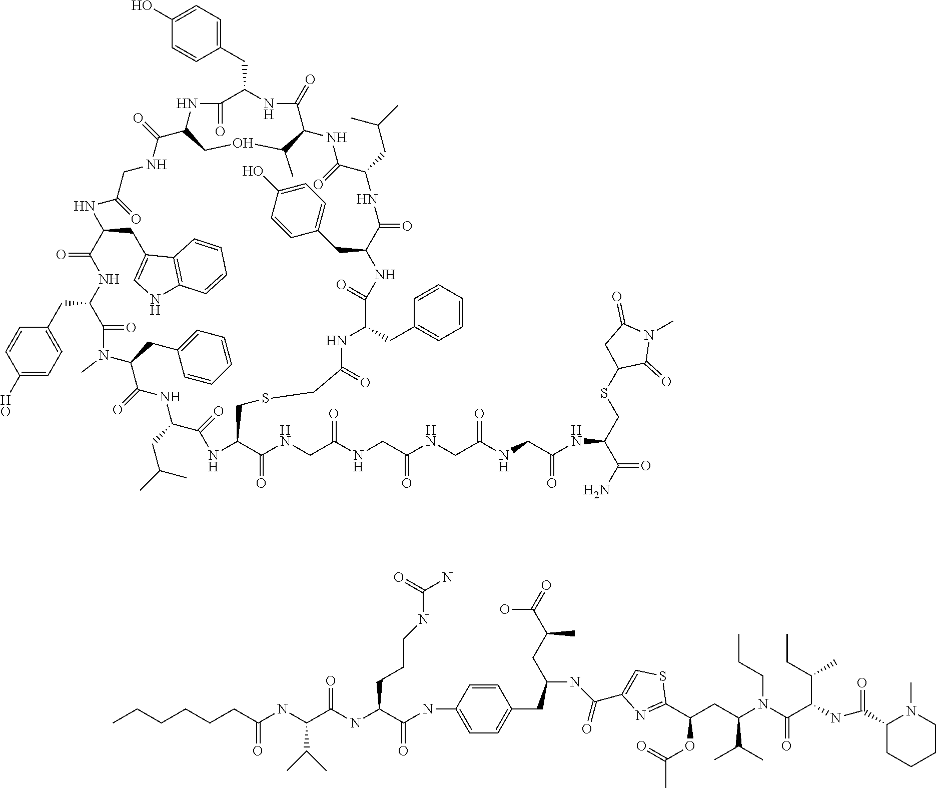

A. Synthesis of Cyclo(AcFYLVYSGWY-[NMe Phe]-LC)-GGGC(Trt)-NH₂

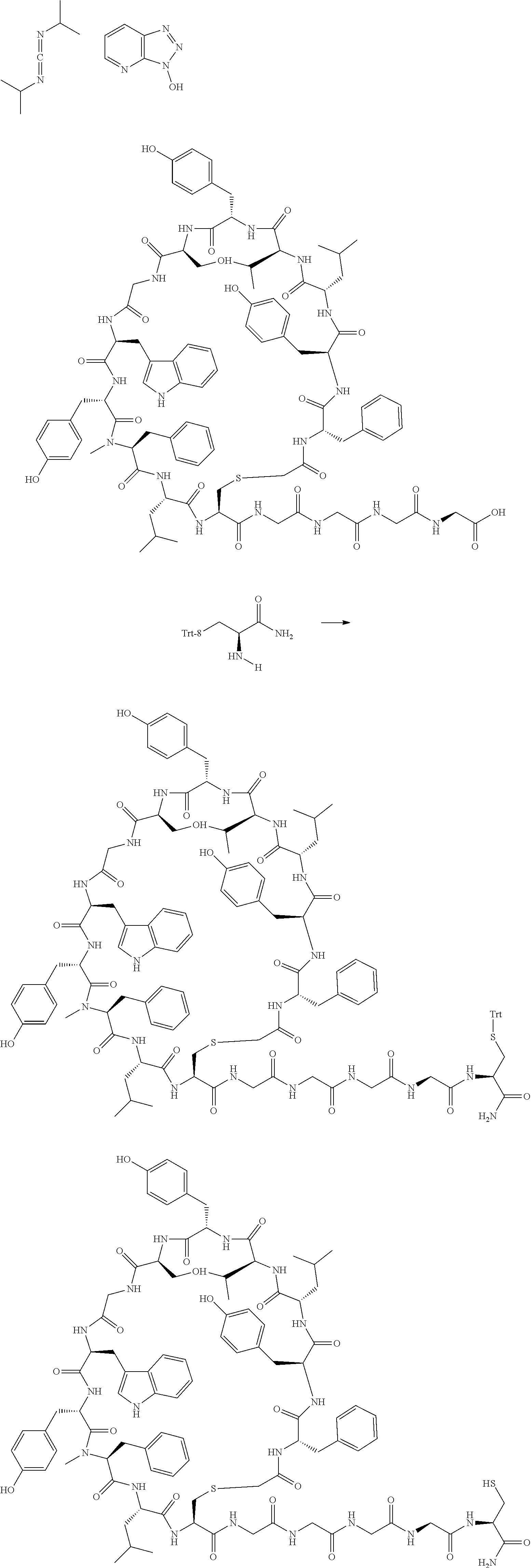

Cyclo(AcFYLVYSGWY-[NMe Phe]-LC)-GGG-OH (1-((3R,6S,9S,12S,15S,21S,24S,27S,30S,33S,36S)-15-((1H-indol-3-yl)methyl)-9,36- dibenzyl-12,24,33-tris(4-hydroxybenzyl)-21-(hydroxymethyl)-6,30-diisobutyl-27-isopropyl-10-methyl-5,8,11,14,17,20,23,26,29,32,35,38-dodecaoxo-1-thia-4,7,10,13,16,19,22,25,28,31,34,37-dodecaazacyclononatriacontan-3-yl)-1,4,7,10-tetraoxo-2,5,8,11-tetraazatridecan-13-oic acid) was synthesized following similar methods described for Example 1000 using Gly-OH preloaded-chlorotrityl resin. A solution of this cyclic peptide (92 mg, 0.05 mmol) and 1-hydroxy-7-azabenzotriazole (34.0 mg, 0.25 mmol) in DMF (2 mL) was added (R)-2-amino-3-(tritylthio)propanamide (91 mg, 0.25 mmol) followed by the addition of N,N′-diisopropylcarbodiimide (38.7 μl, 0.25 mmol). The reaction was shaken overnight. The reaction was purified on a prep HPLC: Gradient Time: 50 min, Flow Rate: 15 ml/min, FRC Collection Time: 0.35 to 50 min; Start % B: 25, Final % B: 80; Solvent A: 0.1% TFA in Water, Solvent B: 0.1% TFA in acetonitrile; Column: Phenom Luna 5u C18(2) 250×21.2 AXIA, 100A Ser. #520221-1; Gradient Collection By: UV; Wavelength: 217, Injection Vol: 2000 uL. Clean fractions were combined and lyophilized to give the desired 1-((3R,6S,9S,12S,15S,21S,24S,27S,30S,33S,36S)-15-((1H-indol-3-yl)methyl)-9,36-dibenzyl-12,24,33-tris(4-hydroxybenzyl)-21-(hydroxymethyl)-6,30-diisobutyl-27-isopropyl-10-methyl-5,8,11,14,17,20,23,26,29,32,35,38-dodecaoxo-1-thia-4,7,10,13,16,19,22,25,28,31,34,37-dodecaazacyclononatriacontan-3-yl)-1,4,7,10-tetraoxo-2,5,8,11-tetraazatridecan-13-oic acid. LCMS: [M−Trt+H]⁺=1945.6.

B. Removal of the trityl group from compound in step A

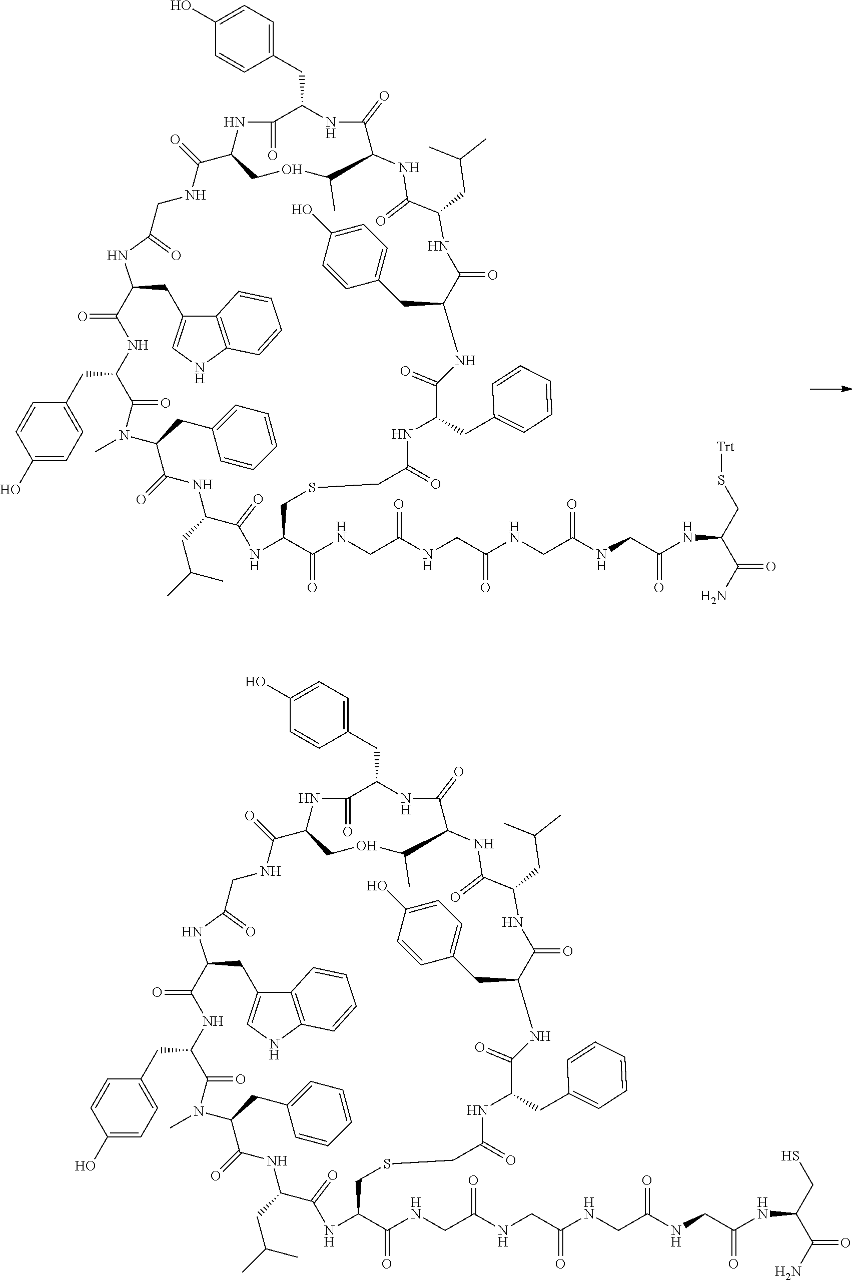

The peptide in step A, (3R,6S,9S,12S,15S,21S,24S,27S,30S,33S,36S)-15-((1H-indol-3-yl)methyl)-9,36-dibenzyl-N-((R)-4-carbamoyl-6,9,12,15-tetraoxo-1,1,1-triphenyl-2-thia-5,8,11,14-tetraazahexadecan-16-yl)-12,24,33-tris(4-hydroxybenzyl)-21-(hydroxymethyl)-6,30-diisobutyl-27-isopropyl-10-methyl- 5,8,11,14,17,20,23,26,29,32,35,38-dodecaoxo-1-thia-4,7,10,13,16,19,22,25,28,31,34,37-dodecaazacyclononatriacontane-3-carboxamide (3.06 mg, 0.0014 mmol) was dissolved in 90% TFA/water (1.5 mL) for 30 min. The reaction was precipitated with ether (8 mL total), cooled in an ice bath for 30 min, centrifuged, and decanted. The white precipitate was dissolved in (1:1) acetonitrile/water (4 mL), transferred to a tared vial, and lyophilized. LCMS: [M+2H]²⁺=973.1, [M+H]⁺=1946.3.

C. Conjugation of a cytotoxic payload to compound in step B.

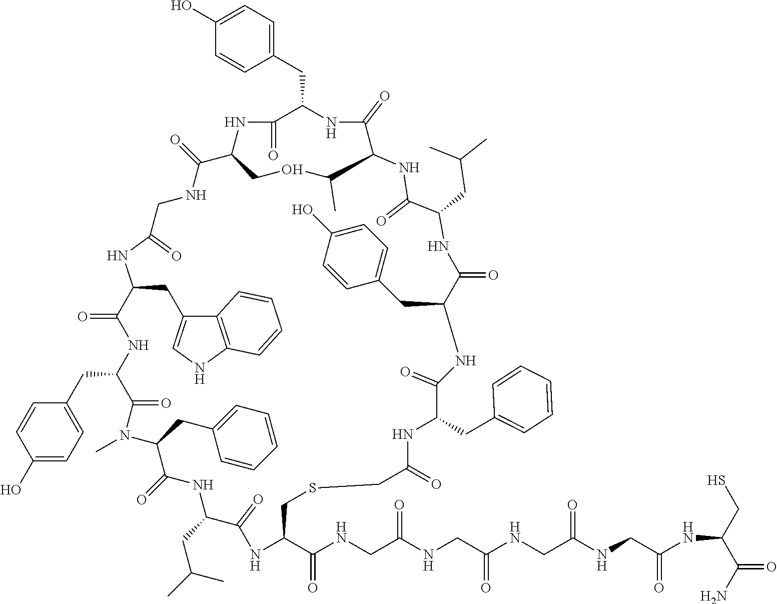

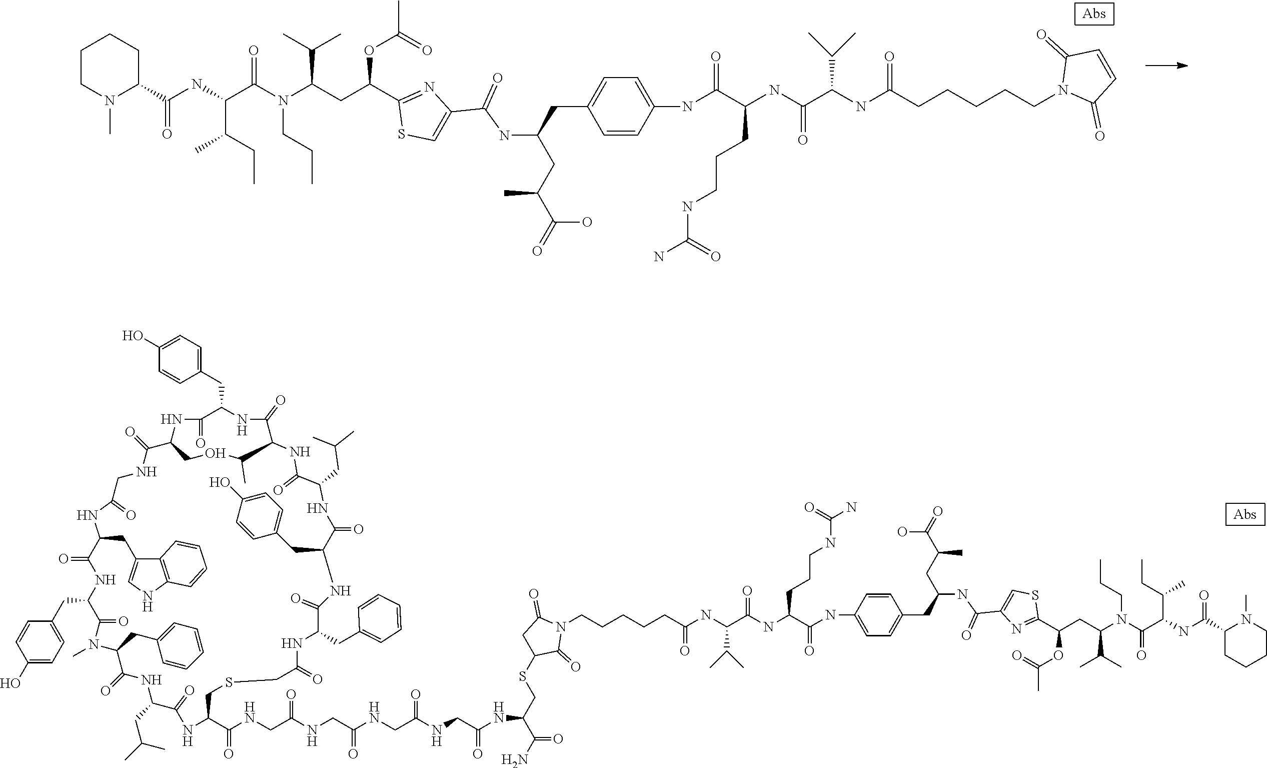

A. Safety precautions: Wear standard PPE for chemistry (lab coat, gloves, safety glasses), and conduct as many manipulations as possible in a chemical fume hood in accordance with the Band 5 SOP. Dispose of waste in accordance with the Band 5 guidelines.

The peptide in step B(3R,6S,9S,12S,15S,21S,24S,27S,30S,33S,36S)-15-((1H-indol-3-yl)methyl)-N-((R)-14-amino-13-(mercaptomethyl)-2,5,8,11,14-pentaoxo-3,6,9,12-tetraazatetradecyl)-9,36-dibenzyl-12,24,33-tris(4-hydroxybenzyl)-21-(hydroxymethyl)-6,30-diisobutyl-27-isopropyl-10-methyl-5,8,11,14,17,20,23,26,29,32,35,38-dodecaoxo-1-thia-4,7,10,13,16,19,22,25,28,31,34,37-dodecaazacyclononatriacontane-3-carboxamide (2.334 mg, 0.0012 mmol) was dissolved in 1×PBS (pH 7.2) (2mL) add in (2S,4R)-4-(2-((1R,3R)-1-acetoxy-4-methyl-3-((2S,3S)-3-methyl-24(R)-1-methylpiperidine-2-carboxamido)-N-propylpentanamido)pentyl)thiazole-4-carboxamido)-5-(4-((S)-2-((S)-2-(6-(2,5-dioxo-2,5-dihydro-1H-pyrrol-1-yl)hexanamido)-3-methylbutanamido)-5-ureidopentanamido)phenyl)-2-methylpentanoic acid (1.47 mg, 1.2 μmol) in acetonitrile (1 mL) and stir at room temperature The reaction was not totally homogeneous, and DMF (200 uL) was added, After 16 h, the reaction was warmed up to approximately 45° C. for 10 min and back to rt and continued stirring at rt for 4 h and lyophilized. The reaction was redissolved in (1:1) acetonitrile/water and purified on a prep-HPLC: Gradient Time: 45 Min, Flow Rate: 15 ml/min, FRC Collection Time: 0.35 to 50 min; Start % B: 15, Final % B: 75; Solvent A: 010% CH₃CN—90% H₂O—0.1% TFA, Solvent B: 90%—CH₃CN—10% H₂—0.1% TFA; Column: Column: 4: LUNA C18 5U 21.2×100 mm. Gradient Collection By: UV; Wavelength: 217, Injection Vol: 2000 uL. Clean fractions were combined and lyophilized. LCMS:[M+4H]⁴⁺=792.1; [M+3H]³⁺=1056.1; [M+2H]²⁺=1583.9.

ROR1 high content cell-based binding assay: Human embryonic kidney 293T cells (293T) expressing human ROR1 with Tet-on system by doxycycline were used (Arctic-488134) for the assessment of inhibition of the binding of fluorescently-labeled competing peptide (“Probe”) that specifically binds to human ROR1. The probe was directly labeled with 5-FAM through BMS internal chemistry protocol (see Example 1300). Cryopreserved vials were thawed in a 37° C. water bath. Cells were washed in assay culture media [DMEM (Life Technologies Inc. Cat. No. 11995-065) supplemented with 10% (v/v) FBS, 1× Penicillin/Streptomycin (Life Technologies Inc. Cat. No. 15140-122) and 0.25 mg/ml dox] and diluted 1.0×105 cells/mL. Cells were seeded into PDL-coated 384 well plates (BD Falcon Cat. No. 356663) at 2,000 cells/20 μL. Plates were allowed to incubate for 10 minutes at room temperature and then transferred to a 37° C./5% CO₂ incubator for overnight incubation. The next day, 125 nL of the test compound was added using an ECHO followed by the addition of 5 μL/well of 5-FAM-labeled probe to cells at a final concentration of 30 nM. Sample were incubated for 1 hour at 37° C. for 1 hour, followed by 3 washes in 100 mL of dPBS. Cells were fixed by adding 8% (w/v) formaldehyde (Sigma Cat. No. 252549), 20 μg/mL Hoechst (Thermo Scientific Cat. No. 62249) in PBS for 20 minutes at room temperature. The plates were washed 3 more times in dPBS and 15 μl of PBS was left in the wells following the last wash. The plates were sealed, and data was using obtained using a CellInsight NXT High Content Screening Platform IC903000 (Thermo Scientific). The 50% effective concentration (IC 50) was calculated using the four-parameter logistic formula y=A+((B−A)/(1+((C/x)\hat{ }D))), where A and B denote minimal and maximal % inhibition, respectively, C is the EC₅₀, D is hill slope and x represent compound concentration.

TABLE 1

| | ROR1 Binding 293T Human |
|---|---|
| Example | HC Cell Based Assay IC50 |
| number | (uM) |
| 1000 | 0.011 |
| 1001 | 0.049 |
| 1002 | 0.013 |
| 1003 | 0.008 |
| 1004 | 0.099 |
| 1005 | 0.030 |
| 1006 | 0.147 |
| 1007 | 0.015 |
| 1008 | 0.036 |
| 1009 | 0.054 |
| 1010 | 0.821 |
| 1011 | 0.168 |
| 1012 | 0.036 |
| 1013 | 0.637 |
| 1014 | 0.707 |
| 1015 | 0.017 |
| 1016 | 0.007 |
| 1017 | 0.014 |
| 1018 | 0.030 |
| 1019 | 0.027 |
| 1020 | 0.018 |
| 1021 | 0.005 |
| 1022 | 0.034 |
| 1023 | 0.079 |
| 1024 | 0.620 |
| 1025 | 0.365 |
| 1026 | 0.059 |
| 1027 | 0.870 |
| 1028 | 0.010 |
| 1029 | 0.255 |
| 1030 | 0.275 |
| 1031 | 0.214 |
| 1032 | 0.392 |
| 1033 | 0.768 |
| 1034 | 0.246 |
| 1035 | 0.059 |
| 1036 | 0.007 |
| 1037 | 0.251 |
| 1038 | 0.023 |
| 1039 | 0.681 |
| 1040 | 0.017 |
| 1041 | 0.012 |
| 1042 | 0.031 |
| 1043 | 0.038 |
| 1044 | 0.038 |
| 1045 | 0.009 |
| 1046 | 0.006 |
| 1047 | 0.243 |
| 1048 | 0.666 |
| 1049 | 0.019 |
| 1050 | 0.015 |
| 1051 | 0.172 |
| 1052 | 0.367 |
| 1053 | 0.760 |
| 1054 | 0.035 |
| 1055 | 0.029 |
| 1056 | 0.740 |
| 1057 | 0.010 |
| 1058 | 0.755 |
| 1059 | 0.093 |
| 1060 | 0.030 |
| 1061 | 0.645 |
| 1062 | 0.020 |
| 1063 | 0.576 |
| 1064 | 0.199 |
| 1065 | 0.022 |
| 1066 | 0.005 |
| 1067 | 0.515 |
| 1068 | 0.453 |
| 1069 | 0.082 |
| 1070 | 0.065 |
| 1071 | 0.072 |
| 1072 | 0.080 |
| 1073 | 0.249 |
| 1074 | 0.122 |
| 1075 | 0.030 |
| 1076 | 0.518 |
| 1077 | 0.002 |
| 1078 | 0.040 |
| 1079 | 0.025 |
| 1080 | 0.217 |
| 1081 | 0.177 |
| 1082 | 0.192 |
| 1083 | 0.022 |
| 1084 | 0.227 |
| 1085 | 0.018 |
| 1086 | 0.364 |
| 1087 | 0.131 |
| 1088 | 0.083 |
| 1089 | 0.077 |
| 1090 | 0.067 |
| 1091 | 0.565 |
| 1092 | 0.043 |
| 1093 | 0.324 |
| 1094 | 0.014 |
| 1095 | 0.011 |
| 1096 | 0.004 |
| 1097 | 0.063 |
| 1100 | 0.121 |
| 1101 | 0.067 |
| 1102 | 0.097 |
| 1103 | 0.923 |
| 1104 | 0.031 |
| 1105 | 0.675 |
| 1106 | 0.483 |
| 1107 | 0.553 |
| 1108 | 0.404 |
| 1109 | 0.574 |
| 1110 | 0.361 |
| 1111 | 0.479 |
| 1112 | 0.147 |
| 1113 | 0.100 |
| 1114 | 0.470 |
| 1115 | 0.094 |
| 1116 | 0.183 |
| 1117 | 0.069 |
| 1118 | 0.151 |
| 1119 | 0.128 |
| 1120 | 0.790 |
| 1121 | 0.378 |
| 1122 | 0.567 |
| 1123 | 0.126 |
| 1124 | 0.035 |
| 1200 | 0.303 |
| 1201 | 0.039 |
| 1202 | 0.067 |
| 1203 | 0.086 |
| 1204 | 0.228 |
| 1205 | 0.346 |

1. A compound of formula (I): 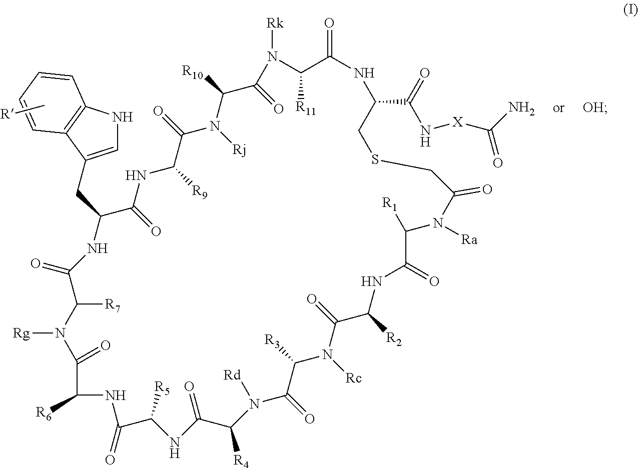
or a pharmaceutically acceptable salt thereof, wherein:
R₁ is selected from C₁-C₆alkyl, hydroxyC₁-C₆alkyl, methoxyC₁-C₆alkyl, aminocarbonylC₁-C₆alkyl, aryl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;
R₂ is selected from hydrogen, C₁-C₆alkyl, arylC₁-C₆alkyl, wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy;
guanidinylC₁-C₆alkyl;
R₃ is selected from C₁-C₆alkyl, aminoC₁-C₆alkyl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo; carboxyC₁-C₆alkyl, guanidinylC₁-C₆alkyl;
R₄ is selected from C₁-C₆alkyl, hydroxyC₁-C₆alkyl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo; carboxyC₁-C₆alkyl, guanidinylC₁-C₆alkyl;
R₅ is selected from hydrogen, C₁-C₆alkyl, hydroxyC₁-C₆alkyl, aminoC₁-C₆alkyl, guanidinylC₁-C₆alkyl, aminocarbonylC₁-C₆alkyl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;
R₆ is selected from hydrogen, C₁-C₆alkyl, hydroxyC₁-C₆alkyl, aminoC₁-C₆alkyl, guanidinylC₁-C₆alkyl, aminocarbonylC₁-C₆alkyl, carboxyC₁-C₆alkyl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl;
R₇ is selected from hydrogen, C₁-C₆alkyl, carboxyC₁-C₆alkyl, aminoC₁-C₆alkyl, guanidinylC₁-C₆alkyl, and heteroarylC₁-C₆alkyl or halo;
R′ is selected from hydrogen, halo, C1-C3alkyl, cyano;
R₉ is selected from C₁-C₆alkyl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;
R₁₀ is selected from C₃-C₆alkyl, arylC₁-C₆alkyl, and heteroarylC₁-C₆alkyl; wherein the aryl part of the arylC₁-C₆alkyl is optionally substituted with one, two, three, four, or five groups independently selected from halo, nitro, amino, C₁-C₆alkyl, aminocarbonyl, hydroxy, aminoC₁-C₆alkyl, aminoC₂-C₆alkoxy, trifluoromethyl, oxotrifluoromethyl, carboxy, cyano, carboxyC₁-C₆alkyl, and carboxyC₁-C₆alkoxy; and wherein the heteroaryl part of the heteroarylC₁-C₆alkyl is optionally substituted with one, two, three, four, or five groups independently selected from C₁-C₆alkyl or halo;
R₁₁ is selected from C₁-C₆alkyl;
Ra is hydrogen or methyl;
Rc is hydrogen or methyl; or Rc and R₃, together with the carbon atom to which they are attached, form a 5-6 ring heterocycle ring, wherein the heterocycle is optionally substituted with one or two groups independently selected from amino, aminocarbonyl, carboxy, carboxyC₁-C₆alkyl, carboxymethoxy, cyano, fluoro, hydroxy, methoxy, methyl, methylcarbonylamino, and trifluoromethyl;
Rd is hydrogen or methyl;
Rg is hydrogen or methyl;
Rj is hydrogen or methyl;
X is selected from —CR₁₃R₁₃′CONHCR₁₄R₁₄′CONHCR₁₅R₁₅′, wherein R₁₃, R₁₄, R₁₅ is independently selected from hydrogen, or any natural or unnatural amino acid side chains, and R₁₃′, R₁₄, ′ and R₁₅′ is interdependently selected from hydrogen or C₁-C₆alkyl; alternatively, X is —(CH₂CH₂O)ₙ—, wherein n=1-13; Alternatively X is —(CH₂CH₂O)ₙ—CONH—CR₁₆R₁₆′; wherein n=1-13 and R₁₆, R₁₆′ are independently selected from hydrogen, aminoC₁-C₄alkyl, carboxyC₁-C₂alkyl or HSC₁-C₂alkyl.

2. The compound of claim 1, or the pharmaceutically acceptable salt thereof, wherein
R₁ is selected from C₁-C₄alkyl, hydroxyC₁-C₃alkyl, aminocarbonylC₁-C2alkyl, aryl, arylC₁-C₂alkyl, and heteroarylC₁-C₂alkyl; wherein the aryl part of the arylC₁-C₂alkyl is phenyl, and is optionally substituted with one, or two groups independently selected from halo, amino, C₁-C₂alkyl, aminocarbonyl, hydroxy, aminoC₁-C₂alkyl, trifluoromethyl, oxotrifluoromethyl, and cyano; and wherein the heteroaryl part of the heteroarylC₁-C₂alkyl is indolyl or imidazole, and is optionally substituted with one or two groups independently selected from methyl, fluoro or chloro,
R₂ is selected from hydrogen, C₁-C₄alkyl, guanidinylC₃-C₄alkyl, arylC₁-C₂alkyl, wherein the aryl part of the arylC₁-C₆alkyl is phenyl and is optionally substituted with one or two groups independently selected from fluoro, amino, C₁-C₃alkyl, hydroxy, aminoC₃-C₄alkyl, or aminocarbonylC₁-C₂alkyl,
R₃ is selected from C₁-C₄alkyl, aminoC₃-C₄alkyl, arylC₁-C₂alkyl, and heteroarylC₁-C₂alkyl; wherein the aryl part of the arylC₁-C₆alkyl is phenyl and is optionally substituted with one or two groups independently selected from fluoro, amino, C₁-C₃alkyl, aminocarbonyl, hydroxy, aminoC₁-C₃alkyl, trifluoromethyl, oxotrifluoromethyl, carboxy, cyano, carboxyC₁-C₂alkyl, and carboxymethoxy; carboxyC1-C2alkyl, guanidinylC₃-C₄alkyl;
alternatively R₃ and Rc, together with the carbon atom to which they are attached, form a pyrrolidine ring, which is optionally substituted with one or two groups independently selected from amino, aminocarbonyl, carboxy, carboxyC₁-C₆ alkyl, carboxymethoxy, cyano, fluoro, hydroxy, methoxy, methyl, methylcarbonylamino, and trifluoromethyl,
R₄ is selected from C₁-C₄alkyl, hydroxyC₁-C₂alkyl, arylC₁-C₂alkyl, and heteroarylC₁-C₂alkyl; wherein the aryl part of the arylC₁-C₆alkyl is optionally substituted with one or two groups independently selected from hydrogen, fluoro, amino, C₁-C₃alkyl, aminocarbonyl, hydroxy, aminoC₁-C₃alkyl, trifluoromethyl, oxotrifluoromethyl, carboxy, cyano, carboxyC₁-C₂alkyl, and carboxymethoxy,
R₅ is hydrogen, C₁-C₄alkyl, hydroxyC₁-C₂alkyl, aminoC₁-C₄alkyl, guanidinylC₃-C₄alkyl, aminocarbonylC₁-C₂alkyl, arylC₁-C₂alkyl, and heteroarylC₁-C₂alkyl; wherein the aryl part of the arylC₁-C₆alkyl is optionally substituted with one or two groups independently selected from hydrogen, fluoro, amino, C₁-C₃alkyl, aminocarbonyl, hydroxy, aminoC₁-C₃alkyl, trifluoromethyl, oxotrifluoromethyl, carboxy, cyano, carboxyC₁-C₂alkyl, and carboxymethoxy,
R₆ is hydrogen, C₁-C₄alkyl, hydroxyC₁-C₂alkyl, aminoC₁-C₄alkyl, guanidinylC₃-C₄alkyl, aminocarbonylC₁-C₂alkyl, phenylC₁-C₂alkyl, and indolylC₁-C₂alkyl, wherein benzyl or indolyl is optionally substituted with one or more groups independently selected from hydroxy, amino, aminocarbonyl and carboxy,
R₇ is selected from hydrogen, C₁-C₄alkyl, aminoC₁-C₄alkyl, guanidinylC₃-C₄alkyl, carboxyC₁-C2alkyl and indolylC₁-C₆alkyl,
R₉ is isopropylmethyl or arylmethyl, wherein the aryl part of the arylmethyl is optionally substituted with one or two groups independently selected from hydroxy, aminocarbonyl, halo, and C₁-C₃alkyl and trifluoromethyl, and
R₁₀ is C₃-C₆alkyl, phenylmethyl or indolylmethyl, wherein the phenyl or indolyl part is optionally substituted with one, two, or three fluoro, methyl, or trifluoromethyl.

3-10. (canceled)

11. The compound of claim 2, or the pharmaceutically acceptable salt thereof, wherein
R₁ is benzyl, indolylmethyl, —CH₂CH₂CONH₂, imidazolylmethyl, isopropylmethyl, and 2-naphthylmethyl,
R₂ is selected from C1-C4alkyl, guanidinylpropyl, 4-hydroxyphenylmethyl,
R₃ is selected from C₁-C₄alkyl, benzyl, 4-hydroxyphenyl, aminobutyl, guanidinylpropyl, alternatively, R₃ and Rc, together with the carbon atom to which they are attached, form a pyrrolidine ring,
R₄ is methyl, isopropyl, benzyl, or indolylmethyl,
R₅ is C₁-C₄alkyl, benzyl, 4-hydroxyphenylmethyl, or hydroxymethyl
R₆ is hydrogen, C₁-C₄alkyl, hydroxymethyl, carboxymethyl, carboxyethyl, guanidinylpropyl, aminocarbonylmethyl, aminocarbonylethyl, aminoC₁-C₄alkyl, benzyl, imidazolyl, indolyl, or 4-hydroxyphenylmethyl,
R₇ is hydrogen, methyl, isopropyl, or guanidinylpropyl,
R₉ is isopropyl, isopropylmethyl, benzyl, or 4-hydroxyphenylmethyl,
R₁₀ is isopropylmethyl, n-butyl, —CH(CH₃)CH₂CH₃, benzyl, 4-hydoxyphenylmethyl, or indolylmethyl, and
R₁₁ is hydrogen, methyl, isopropyl, isopropylmethyl, or n-butyl.

12-20. (canceled)

21. The compound of claim 20, or the pharmaceutically acceptable salt thereof, wherein
R₁ is benzyl or indolylmethyl,
R₂ is 4-hydorxyphenylmethyl.
R₃ is selected from isopropylmethyl, indolylmethyl, guanidinylpropyl, or aminobutyl; alternatively, R₃ and Rc, together with the carbon atom to which they are attached, form the pyrrolidine ring,
R₄ is isopropyl or hydroxymethyl,
R₅ is hydroxymethyl or 4-hydroxyphenylmethyl,
R₆ is selected from hydroxymethyl, guanidinylpropyl, aminocarbonylmethyl, aminocarbonylethyl, carboxyethyl, or 4-hydroxyphenylmethyl,
R₇ is hydrogen, or methyl,
R₉ is 4-hydroxyphenylmethyl,
R₁₀ is selected from n-butyl, isopropylmethyl, or benzyl, and
R₁₁ is n-butyl or isopropylmethyl.

22. The compound of claim 1, or the pharmaceutically acceptable salt thereof, wherein R₁₆ is HS-methyl-, aminoC₁-C₄alkyl, or carboxyC₁-C₂, which can be conjugated to a cytotoxic payload (D) through a linker (L′) to form a general structure (II), one of such examples is illustrated as follows:

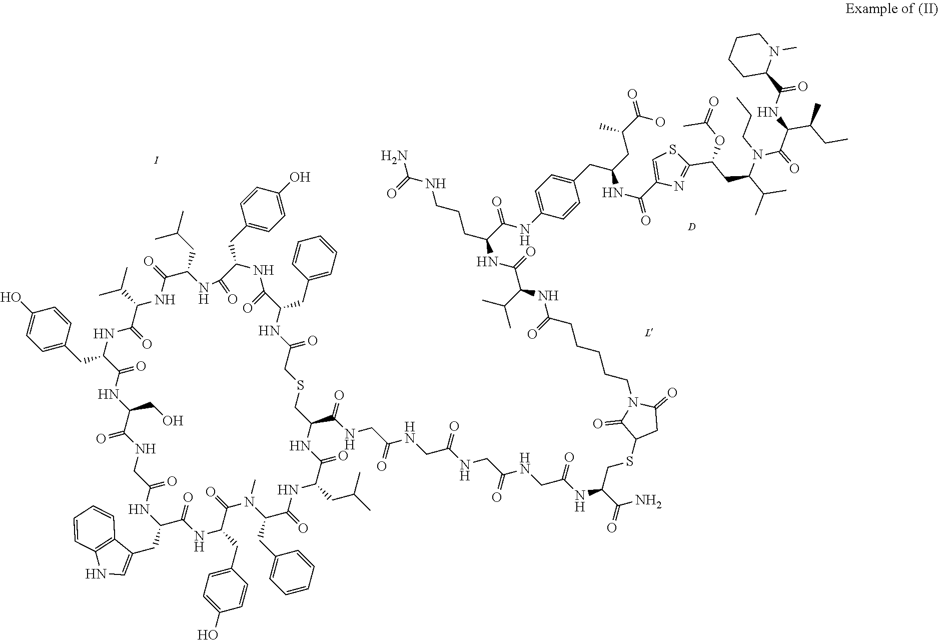

23. The compound of claim 1, or the pharmaceutically acceptable salt thereof, wherein the compound is selected from the compounds listed in Table 1.

24. A pharmaceutical composition comprising a compound of claim 1, or a pharmaceutically acceptable. salt thereof, for use as an inhibitor of tyrosine kinase ROR1 activity in a mammal.

25. A method of enhancing, stimulating, and/or increasing an immune response in a subject in need thereof, wherein the method comprises administering to the subject a therapeutically effective amount of a compound of claim 1 or a pharmaceutically acceptable salt thereof. and optionally a pharmaceutically acceptable excipient.

26. A method of binding the extracellular domain(s) of human ROR1 in a subject, wherein the method comprises administering to the subject a therapeutically effective amount of a compound of claim 1 or a pharmaceutically acceptable salt thereof

27. A compound of formula (F) or (I″), or a pharmaceutically acceptable salt thereof, for use in the treatment of a malignant hyperproliferative disorder.

28. A method of treating a malignant hyperproliferative disorder, wherein the method comprises administering to the subject a therapeutically effective amount of a compound of claim 1 or a pharmaceutically acceptable salt thereof.

29. The method of claim 28 wherein the malignant hyperproliferative disorder is hematological tumors and solid tumors.

30. The method of claim 29 wherein the hematological tumors are chronic lymphocytic leukemia (CLL), acute myeloid leukemia (AML), acute lymphoblastic leukemia (ALL) or mantle cell lymphoma and the solid tumors are lung, ovarian, breast or pancreatic tumors.

31. (canceled)
